SEQUENCE LISTING (398 sequences)

SEQ ID NO: 1 - protein, 350 aa, Staphylococcus aureus
MTKHYLNSKYQSEQRSSAMKKITMGTASIILGSLVYIGADSQQVNAATEATNATNNQSTQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAKPNNVKPVQPKPAQPKTPTEQTKPVQPKVEKVKPTVTTTSKVEDNHSTKVVSTDTTKDQTKTQTAHTVKTAQTAQEQNKVQTPVKDVATAKSESNNQAVSDNKSQQTNKVTKHNETPKQASKAKELPKTGLTSVDNFISTVAFATLALLGSLSLLLFKRKESK

SEQ ID NO: 2 - protein, 645 aa, Staphylococcus aureus
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKAVKPAAKATNNTYPILNQELREAIKNPAIKDKDHSAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 3 - protein, 123 aa, Artificial Sequence
  IsdA peptide
SQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLA

SEQ ID NO: 4 - protein, 62 aa, Artificial Sequence
  IsdA peptide
SQATSQPINFQVQKDGSSEKSHMDDYMQHPGKVIKQNNKYYFQTVLNNASFWKEYKFYNANN

SEQ ID NO: 5 - protein, 61 aa, Artificial Sequence
  IsdA peptide
QELATTVVNDNKKADTRTINVAVEPGYKSLTTKVHIVVPQINYNHRYTTHLEFEKAIPTLA

SEQ ID NO: 6 - protein, 20 aa, Artificial Sequence
  IsdA peptide
SQATSQPINFQVQKDGSSEK

SEQ ID NO: 7 - protein, 20 aa, Artificial Sequence
  IsdA peptide
QVQKDGSSEKSHMDDYMQHP

SEQ ID NO: 8 - protein, 20 aa, Artificial Sequence
  IsdA peptide
SHMDDYMQHPGKVIKQNNKY

SEQ ID NO: 9 - protein, 20 aa, Artificial Sequence
  IsdA peptide
GKVIKQNNKYYFQTVLNNAS

SEQ ID NO: 10 - protein, 20 aa, Artificial Sequence
  IsdA peptide
YFQTVLNNASFWKEYKFYNA

SEQ ID NO: 11 - protein, 20 aa, Artificial Sequence
  IsdA peptide
FWKEYKFYNANNQELATTVV

SEQ ID NO: 12 - protein, 20 aa, Artificial Sequence
  IsdA peptide
NNQELATTVVNDNKKADTRT

SEQ ID NO: 13 - protein, 20 aa, Artificial Sequence
  IsdA peptide
NDNKKADTRTINVAVEPGYK

SEQ ID NO: 14 - protein, 20 aa, Artificial Sequence
  IsdA peptide
INVAVEPGYKSLTTKVHIVV

SEQ ID NO: 15 - protein, 20 aa, Artificial Sequence
  IsdA peptide
SLTTKVHIVVPQINYNHRYT

SEQ ID NO: 16 - protein, 23 aa, Artificial Sequence
  IsdA peptide
PQINYNHRYTTHLEFEKAIPTLA

SEQ ID NO: 17 - protein, 10 aa, Artificial Sequence
  IsdA peptide
SQATSQPINF

SEQ ID NO: 18 - protein, 10 aa, Artificial Sequence
  IsdA peptide
QATSQPINFQ

SEQ ID NO: 19 - protein, 10 aa, Artificial Sequence
  IsdA peptide
ATSQPINFQV

SEQ ID NO: 20 - protein, 10 aa, Artificial Sequence
  IsdA peptide
TSQPINFQVQ

SEQ ID NO: 21 - protein, 10 aa, Artificial Sequence
  IsdA peptide
SQPINFQVQK

SEQ ID NO: 22 - protein, 10 aa, Artificial Sequence
  IsdA peptide
QPINFQVQKD

SEQ ID NO: 23 - protein, 10 aa, Artificial Sequence
  IsdA peptide
PINFQVQKDG

SEQ ID NO: 24 - protein, 10 aa, Artificial Sequence
  IsdA peptide
INFQVQKDGS

SEQ ID NO: 25 - protein, 10 aa, Artificial Sequence
  IsdA peptide
FQVQKDGSSE

SEQ ID NO: 26 - protein, 10 aa, Artificial Sequence
  IsdA peptide
QVQKDGSSEK

SEQ ID NO: 27 - protein, 10 aa, Artificial Sequence
  IsdA peptide
VQKDGSSEKS

SEQ ID NO: 28 - protein, 10 aa, Artificial Sequence
  IsdA peptide
QKDGSSEKSH

SEQ ID NO: 29 - protein, 10 aa, Artificial Sequence
  IsdA peptide
KDGSSEKSHM

SEQ ID NO: 30 - protein, 10 aa, Artificial Sequence
  IsdA peptide
DGSSEKSHMD

SEQ ID NO: 31 - protein, 10 aa, Artificial Sequence
  IsdA peptide
GSSEKSHMDD

SEQ ID NO: 32 - protein, 10 aa, Artificial Sequence
  IsdA peptide
SSEKSHMDDY

SEQ ID NO: 33 - protein, 10 aa, Artificial Sequence
  IsdA peptide
SEKSHMDDYM

SEQ ID NO: 34 - protein, 10 aa, Artificial Sequence
  IsdA peptide
EKSHMDDYMQ

SEQ ID NO: 35 - protein, 10 aa, Artificial Sequence
  IsdA peptide
KSHMDDYMQH

SEQ ID NO: 36 - protein, 10 aa, Artificial Sequence
  IsdA peptide
SHMDDYMQHP

SEQ ID NO: 37 - protein, 10 aa, Artificial Sequence
  IsdA peptide
HMDDYMQHPG

SEQ ID NO: 38 - protein, 10 aa, Artificial Sequence
  IsdA peptide
MDDYMQHPGK

SEQ ID NO: 39 - protein, 10 aa, Artificial Sequence
  IsdA peptide
DDYMQHPGKV

SEQ ID NO: 40 - protein, 10 aa, Artificial Sequence
  IsdA peptide
DYMQHPGKVI

SEQ ID NO: 41 - protein, 10 aa, Artificial Sequence
  IsdA peptide
YMQHPGKVIK

SEQ ID NO: 42 - protein, 10 aa, Artificial Sequence
  IsdA peptide
MQHPGKVIKQ

SEQ ID NO: 43 - protein, 10 aa, Artificial Sequence
  IsdA peptide
QHPGKVIKQN

SEQ ID NO: 44 - protein, 10 aa, Artificial Sequence
  IsdA peptide
HPGKVIKQNN

SEQ ID NO: 45 - protein, 10 aa, Artificial Sequence
  IsdA peptide
PGKVIKQNNK

SEQ ID NO: 46 - protein, 10 aa, Artificial Sequence
  IsdA peptide
GKVIKQNNKY

SEQ ID NO: 47 - protein, 10 aa, Artificial Sequence
  IsdA peptide
KVIKQNNKYY

SEQ ID NO: 48 - protein, 10 aa, Artificial Sequence
  IsdA peptide
VIKQNNKYYF

SEQ ID NO: 49 - protein, 10 aa, Artificial Sequence
  IsdA peptide
IKQNNKYYFQ

SEQ ID NO: 50 - protein, 10 aa, Artificial Sequence
  IsdA peptide
KQNNKYYFQT

SEQ ID NO: 51 - protein, 10 aa, Artificial Sequence
  IsdA peptide
QNNKYYFQTV

SEQ ID NO: 52 - protein, 10 aa, Artificial Sequence
  IsdA peptide
NNKYYFQTVL

SEQ ID NO: 53 - protein, 10 aa, Artificial Sequence
  IsdA peptide
NKYYFQTVLN

SEQ ID NO: 54 - protein, 10 aa, Artificial Sequence
  IsdA peptide
KYYFQTVLNN

SEQ ID NO: 55 - protein, 10 aa, Artificial Sequence
  IsdA peptide
YYFQTVLNNA

SEQ ID NO: 56 - protein, 10 aa, Artificial Sequence
  IsdA peptide
YFQTVLNNAS

SEQ ID NO: 57 - protein, 10 aa, Artificial Sequence
  IsdA peptide
FQTVLNNASF

SEQ ID NO: 58 - protein, 10 aa, Artificial Sequence
  IsdA peptide
QTVLNNASFW

SEQ ID NO: 59 - protein, 10 aa, Artificial Sequence
  IsdA peptide
TVLNNASFWK

SEQ ID NO: 60 - protein, 10 aa, Artificial Sequence
  IsdA peptide
VLNNASFWKE

SEQ ID NO: 61 - protein, 10 aa, Artificial Sequence
  IsdA peptide
LNNASFWKEY

SEQ ID NO: 62 - protein, 10 aa, Artificial Sequence
  IsdA peptide
NNASFWKEYK

SEQ ID NO: 63 - protein, 10 aa, Artificial Sequence
  IsdA peptide
NASFWKEYKF

SEQ ID NO: 64 - protein, 10 aa, Artificial Sequence
  IsdA peptide
ASFWKEYKFY

SEQ ID NO: 65 - protein, 10 aa, Artificial Sequence
  IsdA peptide
SFWKEYKFYN

SEQ ID NO: 66 - protein, 10 aa, Artificial Sequence
  IsdA peptide
FWKEYKFYNA

SEQ ID NO: 67 - protein, 10 aa, Artificial Sequence
  IsdA peptide
WKEYKFYNAN

SEQ ID NO: 68 - protein, 10 aa, Artificial Sequence
  IsdA peptide
KEYKFYNANN

SEQ ID NO: 69 - protein, 10 aa, Artificial Sequence
  IsdA peptide
EYKFYNANNQ

SEQ ID NO: 70 - protein, 10 aa, Artificial Sequence
  IsdA peptide
YKFYNANNQE

SEQ ID NO: 71 - protein, 10 aa, Artificial Sequence
  IsdA peptide
KFYNANNQEL

SEQ ID NO: 72 - protein, 10 aa, Artificial Sequence
  IsdA peptide
FYNANNQELA

SEQ ID NO: 73 - protein, 10 aa, Artificial Sequence
  IsdA peptide
YNANNQELAT

SEQ ID NO: 74 - protein, 10 aa, Artificial Sequence
  IsdA peptide
NANNQELATT

SEQ ID NO: 75 - protein, 10 aa, Artificial Sequence
  IsdA peptide
ANNQELATTV

SEQ ID NO: 76 - protein, 10 aa, Artificial Sequence
  IsdA peptide
NNQELATTVV

SEQ ID NO: 77 - protein, 10 aa, Artificial Sequence
  IsdA peptide
NQELATTVVN

SEQ ID NO: 78 - protein, 10 aa, Artificial Sequence
  IsdA peptide
QELATTVVND

SEQ ID NO: 79 - protein, 10 aa, Artificial Sequence
  IsdA peptide
ELATTVVNDN

SEQ ID NO: 80 - protein, 10 aa, Artificial Sequence
  IsdA peptide
LATTVVNDNK

SEQ ID NO: 81 - protein, 10 aa, Artificial Sequence
  IsdA peptide
ATTVVNDNKK

SEQ ID NO: 82 - protein, 10 aa, Artificial Sequence
  IsdA peptide
TTVVNDNKKA

SEQ ID NO: 83 - protein, 10 aa, Artificial Sequence
  IsdA peptide
TVVNDNKKAD

SEQ ID NO: 84 - protein, 10 aa, Artificial Sequence
  IsdA peptide
VVNDNKKADT

SEQ ID NO: 85 - protein, 10 aa, Artificial Sequence
  IsdA peptide
VNDNKKADTR

SEQ ID NO: 86 - protein, 10 aa, Artificial Sequence
  IsdA peptide
NDNKKADTRT

SEQ ID NO: 87 - protein, 10 aa, Artificial Sequence
  IsdA peptide
DNKKADTRTI

SEQ ID NO: 88 - protein, 10 aa, Artificial Sequence
  IsdA peptide
NKKADTRTIN

SEQ ID NO: 89 - protein, 10 aa, Artificial Sequence
  IsdA peptide
KKADTRTINV

SEQ ID NO: 90 - protein, 10 aa, Artificial Sequence
  IsdA peptide
KADTRTINVA

SEQ ID NO: 91 - protein, 10 aa, Artificial Sequence
  IsdA peptide
ADTRTINVAV

SEQ ID NO: 92 - protein, 10 aa, Artificial Sequence
  IsdA peptide
DTRTINVAVE

SEQ ID NO: 93 - protein, 10 aa, Artificial Sequence
  IsdA peptide
TRTINVAVEP

SEQ ID NO: 94 - protein, 10 aa, Artificial Sequence
  IsdA peptide
RTINVAVEPG

SEQ ID NO: 95 - protein, 10 aa, Artificial Sequence
  IsdA peptide
TINVAVEPGY

SEQ ID NO: 96 - protein, 10 aa, Artificial Sequence
  IsdA peptide
INVAVEPGYK

SEQ ID NO: 97 - protein, 10 aa, Artificial Sequence
  IsdA peptide
NVAVEPGYKS

SEQ ID NO: 98 - protein, 10 aa, Artificial Sequence
  IsdA peptide
VAVEPGYKSL

SEQ ID NO: 99 - protein, 10 aa, Artificial Sequence
  IsdA peptide
AVEPGYKSLT

SEQ ID NO: 100 - protein, 10 aa, Artificial Sequence
  IsdA peptide
EPGYKSLTTK

SEQ ID NO: 101 - protein, 10 aa, Artificial Sequence
  IsdA peptide
PGYKSLTTKV

SEQ ID NO: 102 - protein, 10 aa, Artificial Sequence
  IsdA peptide
GYKSLTTKVH

SEQ ID NO: 103 - protein, 10 aa, Artificial Sequence
  IsdA peptide
YKSLTTKVHI

SEQ ID NO: 104 - protein, 10 aa, Artificial Sequence
  IsdA peptide
KSLTTKVHIV

SEQ ID NO: 105 - protein, 10 aa, Artificial Sequence
  IsdA peptide
SLTTKVHIVV

SEQ ID NO: 106 - protein, 10 aa, Artificial Sequence
  IsdA peptide
LTTKVHIVVP

SEQ ID NO: 107 - protein, 10 aa, Artificial Sequence
  IsdA peptide
TTKVHIVVPQ

SEQ ID NO: 108 - protein, 10 aa, Artificial Sequence
  IsdA peptide
KVHIVVPQIN

SEQ ID NO: 109 - protein, 10 aa, Artificial Sequence
  IsdA peptide
VHIVVPQINY

SEQ ID NO: 110 - protein, 10 aa, Artificial Sequence
  IsdA peptide
HIVVPQINYN

SEQ ID NO: 111 - protein, 10 aa, Artificial Sequence
  IsdA peptide
IVVPQINYNH

SEQ ID NO: 112 - protein, 10 aa, Artificial Sequence
  IsdA peptide
VVPQINYNHR

SEQ ID NO: 113 - protein, 10 aa, Artificial Sequence
  IsdA peptide
VPQINYNHRY

SEQ ID NO: 114 - protein, 10 aa, Artificial Sequence
  IsdA peptide
PQINYNHRYT

SEQ ID NO: 115 - protein, 10 aa, Artificial Sequence
  IsdA peptide
QINYNHRYTT

SEQ ID NO: 116 - protein, 10 aa, Artificial Sequence
  IsdA peptide
INYNHRYTTH

SEQ ID NO: 117 - protein, 10 aa, Artificial Sequence
  IsdA peptide
NYNHRYTTHL

SEQ ID NO: 118 - protein, 10 aa, Artificial Sequence
  IsdA peptide
YNHRYTTHLE

SEQ ID NO: 119 - protein, 10 aa, Artificial Sequence
  IsdA peptide
NHRYTTHLEF

SEQ ID NO: 120 - protein, 10 aa, Artificial Sequence
  IsdA peptide
HRYTTHLEFE

SEQ ID NO: 121 - protein, 10 aa, Artificial Sequence
  IsdA peptide
RYTTHLEFEK

SEQ ID NO: 122 - protein, 10 aa, Artificial Sequence
  IsdA peptide
YTTHLEFEKA

SEQ ID NO: 123 - protein, 10 aa, Artificial Sequence
  IsdA peptide
TTHLEFEKAI

SEQ ID NO: 124 - protein, 10 aa, Artificial Sequence
  IsdA peptide
THLEFEKAIP

SEQ ID NO: 125 - protein, 10 aa, Artificial Sequence
  IsdA peptide
HLEFEKAIPT

SEQ ID NO: 126 - protein, 10 aa, Artificial Sequence
  IsdA peptide
LEFEKAIPTL

SEQ ID NO: 127 - protein, 10 aa, Artificial Sequence
  IsdA peptide
EFEKAIPTLA

SEQ ID NO: 128 - protein, 327 aa, Artificial Sequence
  IsdB peptide
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKAVKPAAKATNNTYPILNQELREAIKNPAIKDKDHSAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKS

SEQ ID NO: 129 - protein, 120 aa, Artificial Sequence
  IsdB peptide
SAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNS

SEQ ID NO: 130 - protein, 60 aa, Artificial Sequence
  IsdB peptide
SAPNSRPIDFEMKKENGEQQFYHYASSVKPARVIFTDSKPEIELGLQSGQFWRKFEVYEG

SEQ ID NO: 131 - protein, 60 aa, Artificial Sequence
  IsdB peptide
DKKLPIKLVSYDTVKDYAYIRFSVSNGTKAVKIVSSTHFNNKEEKYDYTLMEFAQPIYNS

SEQ ID NO: 132 - protein, 20 aa, Artificial Sequence
  IsdB peptide
SAPNSRPIDFEMKKENGEQQ

SEQ ID NO: 133 - protein, 20 aa, Artificial Sequence
  IsdB peptide
DFEMKKENGEQQFYHYASSV

SEQ ID NO: 134 - protein, 20 aa, Artificial Sequence
  IsdB peptide
SVKPARVIFTDSKPEIELGL

SEQ ID NO: 135 - protein, 20 aa, Artificial Sequence
  IsdB peptide
FYHYASSVKPARVIFTDSKP

SEQ ID NO: 136 - protein, 20 aa, Artificial Sequence
  IsdB peptide
EIELGLQSGQFWRKFEVYEG

SEQ ID NO: 137 - protein, 20 aa, Artificial Sequence
  IsdB peptide
KPEIELGLQSGQFWRKFEVY

SEQ ID NO: 138 - protein, 20 aa, Artificial Sequence
  IsdB peptide
DKKLPIKLVSYDTVKDYAYI

SEQ ID NO: 139 - protein, 20 aa, Artificial Sequence
  IsdB peptide
RFSVSNGTKAVKIVSSTHFN

SEQ ID NO: 140 - protein, 20 aa, Artificial Sequence
  IsdB peptide
NKEEKYDYTLMEFAQPIYNS

SEQ ID NO: 141 - protein, 20 aa, Artificial Sequence
  IsdB peptide
YDTVKDYAYIRFSVSNGTKA

SEQ ID NO: 142 - protein, 20 aa, Artificial Sequence
  IsdB peptide
VKIVSSTHFNNKEEKYDYTL

SEQ ID NO: 143 - protein, 10 aa, Artificial Sequence
  IsdB peptide
SAPNSRPIDF

SEQ ID NO: 144 - protein, 10 aa, Artificial Sequence
  IsdB peptide
APNSRPIDFE

SEQ ID NO: 145 - protein, 10 aa, Artificial Sequence
  IsdB peptide
PNSRPIDFEM

SEQ ID NO: 146 - protein, 10 aa, Artificial Sequence
  IsdB peptide
NSRPIDFEMK

SEQ ID NO: 147 - protein, 10 aa, Artificial Sequence
  IsdB peptide
SRPIDFEMKK

SEQ ID NO: 148 - protein, 11 aa, Artificial Sequence
  IsdB peptide
RPIDFEMKKEN

SEQ ID NO: 149 - protein, 10 aa, Artificial Sequence
  IsdB peptide
IDFEMKKENG

SEQ ID NO: 150 - protein, 10 aa, Artificial Sequence
  IsdB peptide
DFEMKKENGE

SEQ ID NO: 151 - protein, 10 aa, Artificial Sequence
  IsdB peptide
FEMKKENGEQ

SEQ ID NO: 152 - protein, 10 aa, Artificial Sequence
  IsdB peptide
EMKKENGEQQ

SEQ ID NO: 153 - protein, 10 aa, Artificial Sequence
  IsdB peptide
MKKENGEQQF

SEQ ID NO: 154 - protein, 10 aa, Artificial Sequence
  IsdB peptide
KKENGEQQFY

SEQ ID NO: 155 - protein, 10 aa, Artificial Sequence
  IsdB peptide
KENGEQQFYH

SEQ ID NO: 156 - protein, 10 aa, Artificial Sequence
  IsdB peptide
ENGEQQFYHY

SEQ ID NO: 157 - protein, 10 aa, Artificial Sequence
  IsdB peptide
NGEQQFYHYA

SEQ ID NO: 158 - protein, 10 aa, Artificial Sequence
  IsdB peptide
GEQQFYHYAS

SEQ ID NO: 159 - protein, 10 aa, Artificial Sequence
  IsdB peptide
EQQFYHYASS

SEQ ID NO: 160 - protein, 10 aa, Artificial Sequence
  IsdB peptide
QQFYHYASSV

SEQ ID NO: 161 - protein, 10 aa, Artificial Sequence
  IsdB peptide
QFYHYASSVK

SEQ ID NO: 162 - protein, 10 aa, Artificial Sequence
  IsdB peptide
YHYASSVKPA

SEQ ID NO: 163 - protein, 10 aa, Artificial Sequence
  IsdB peptide
HYASSVKPAR

SEQ ID NO: 164 - protein, 10 aa, Artificial Sequence
  IsdB peptide
YASSVKPARV

SEQ ID NO: 165 - protein, 10 aa, Artificial Sequence
  IsdB peptide
ASSVKPARVI

SEQ ID NO: 166 - protein, 10 aa, Artificial Sequence
  IsdB peptide
SSVKPARVIF

SEQ ID NO: 167 - protein, 10 aa, Artificial Sequence
  IsdB peptide
SVKPARVIFT

SEQ ID NO: 168 - protein, 10 aa, Artificial Sequence
  IsdB peptide
VKPARVIFTD

SEQ ID NO: 169 - protein, 10 aa, Artificial Sequence
  IsdB peptide
KPARVIFTDS

SEQ ID NO: 170 - protein, 10 aa, Artificial Sequence
  IsdB peptide
PARVIFTDSK

SEQ ID NO: 171 - protein, 10 aa, Artificial Sequence
  IsdB peptide
ARVIFTDSKP

SEQ ID NO: 172 - protein, 10 aa, Artificial Sequence
  IsdB peptide
RVIFTDSKPE

SEQ ID NO: 173 - protein, 10 aa, Artificial Sequence
  IsdB peptide
VIFTDSKPEI

SEQ ID NO: 174 - protein, 10 aa, Artificial Sequence
  IsdB peptide
IFTDSKPEIE

SEQ ID NO: 175 - protein, 10 aa, Artificial Sequence
  IsdB peptide
FTDSKPEIEL

SEQ ID NO: 176 - protein, 10 aa, Artificial Sequence
  IsdB peptide
TDSKPEIELG

SEQ ID NO: 177 - protein, 10 aa, Artificial Sequence
  IsdB peptide
DSKPEIELGL

SEQ ID NO: 178 - protein, 10 aa, Artificial Sequence
  IsdB peptide
SKPEIELGLQ

SEQ ID NO: 179 - protein, 10 aa, Artificial Sequence
  IsdB peptide
KPEIELGLQS

SEQ ID NO: 180 - protein, 10 aa, Artificial Sequence
  IsdB peptide
PEIELGLQSG

SEQ ID NO: 181 - protein, 10 aa, Artificial Sequence
  IsdB peptide
EIELGLQSGQ

SEQ ID NO: 182 - protein, 10 aa, Artificial Sequence
  IsdB peptide
IELGLQSGQF

SEQ ID NO: 183 - protein, 10 aa, Artificial Sequence
  IsdB peptide
ELGLQSGQFW

SEQ ID NO: 184 - protein, 10 aa, Artificial Sequence
  IsdB peptide
LGLQSGQFWR

SEQ ID NO: 185 - protein, 10 aa, Artificial Sequence
  IsdB peptide
GLQSGQFWRK

SEQ ID NO: 186 - protein, 10 aa, Artificial Sequence
  IsdB peptide
LQSGQFWRKF

SEQ ID NO: 187 - protein, 10 aa, Artificial Sequence
  IsdB peptide
QSGQFWRKFE

SEQ ID NO: 188 - protein, 10 aa, Artificial Sequence
  IsdB peptide
SGQFWRKFEV

SEQ ID NO: 189 - protein, 10 aa, Artificial Sequence
  IsdB peptide
GQFWRKFEVY

SEQ ID NO: 190 - protein, 10 aa, Artificial Sequence
  IsdB peptide
QFWRKFEVYE

SEQ ID NO: 191 - protein, 10 aa, Artificial Sequence
  IsdB peptide
FWRKFEVYEG

SEQ ID NO: 192 - protein, 10 aa, Artificial Sequence
  IsdB peptide
WRKFEVYEGD

SEQ ID NO: 193 - protein, 10 aa, Artificial Sequence
  IsdB peptide
RKFEVYEGDK

SEQ ID NO: 194 - protein, 10 aa, Artificial Sequence
  IsdB peptide
KFEVYEGDKK

SEQ ID NO: 195 - protein, 10 aa, Artificial Sequence
  IsdB peptide
FEVYEGDKKL

SEQ ID NO: 196 - protein, 10 aa, Artificial Sequence
  IsdB peptide
EVYEGDKKLP

SEQ ID NO: 197 - protein, 10 aa, Artificial Sequence
  IsdB peptide
VYEGDKKLPI

SEQ ID NO: 198 - protein, 10 aa, Artificial Sequence
  IsdB peptide
YEGDKKLPIK

SEQ ID NO: 199 - protein, 10 aa, Artificial Sequence
  IsdB peptide
EGDKKLPIKL

SEQ ID NO: 200 - protein, 10 aa, Artificial Sequence
  IsdB peptide
GDKKLPIKLV

SEQ ID NO: 201 - protein, 10 aa, Artificial Sequence
  IsdB peptide
DKKLPIKLVS

SEQ ID NO: 202 - protein, 10 aa, Artificial Sequence
  IsdB peptide
KKLPIKLVSY

SEQ ID NO: 203 - protein, 10 aa, Artificial Sequence
  IsdB peptide
KLPIKLVSYD

SEQ ID NO: 204 - protein, 10 aa, Artificial Sequence
  IsdB peptide
LPIKLVSYDT

SEQ ID NO: 205 - protein, 10 aa, Artificial Sequence
  IsdB peptide
PIKLVSYDTV

SEQ ID NO: 206 - protein, 10 aa, Artificial Sequence
  IsdB peptide
IKLVSYDTVK

SEQ ID NO: 207 - protein, 10 aa, Artificial Sequence
  IsdB peptide
KLVSYDTVKD

SEQ ID NO: 208 - protein, 10 aa, Artificial Sequence
  IsdB peptide
LVSYDTVKDY

SEQ ID NO: 209 - protein, 10 aa, Artificial Sequence
  IsdB peptide
VSYDTVKDYA

SEQ ID NO: 210 - protein, 10 aa, Artificial Sequence
  IsdB peptide
SYDTVKDYAY

SEQ ID NO: 211 - protein, 10 aa, Artificial Sequence
  IsdB peptide
YDTVKDYAYI

SEQ ID NO: 212 - protein, 10 aa, Artificial Sequence
  IsdB peptide
DTVKDYAYIR

SEQ ID NO: 213 - protein, 10 aa, Artificial Sequence
  IsdB peptide
TVKDYAYIRF

SEQ ID NO: 214 - protein, 10 aa, Artificial Sequence
  IsdB peptide
VKDYAYIRFS

SEQ ID NO: 215 - protein, 10 aa, Artificial Sequence
  IsdB peptide
KDYAYIRFSV

SEQ ID NO: 216 - protein, 10 aa, Artificial Sequence
  IsdB peptide
DYAYIRFSVS

SEQ ID NO: 217 - protein, 10 aa, Artificial Sequence
  IsdB peptide
YAYIRFSVSN

SEQ ID NO: 218 - protein, 10 aa, Artificial Sequence
  IsdB peptide
AYIRFSVSNG

SEQ ID NO: 219 - protein, 10 aa, Artificial Sequence
  IsdB peptide
YIRFSVSNGT

SEQ ID NO: 220 - protein, 10 aa, Artificial Sequence
  IsdB peptide
IRFSVSNGTK

SEQ ID NO: 221 - protein, 10 aa, Artificial Sequence
  IsdB peptide
RFSVSNGTKA

SEQ ID NO: 222 - protein, 10 aa, Artificial Sequence
  IsdB peptide
FSVSNGTKAV

SEQ ID NO: 223 - protein, 10 aa, Artificial Sequence
  IsdB peptide
SVSNGTKAVK

SEQ ID NO: 224 - protein, 10 aa, Artificial Sequence
  IsdB peptide
VSNGTKAVKI

SEQ ID NO: 225 - protein, 10 aa, Artificial Sequence
  IsdB peptide
SNGTKAVKIV

SEQ ID NO: 226 - protein, 10 aa, Artificial Sequence
  IsdB peptide
NGTKAVKIVS

SEQ ID NO: 227 - protein, 10 aa, Artificial Sequence
  IsdB peptide
GTKAVKIVSS

SEQ ID NO: 228 - protein, 10 aa, Artificial Sequence
  IsdB peptide
TKAVKIVSST

SEQ ID NO: 229 - protein, 10 aa, Artificial Sequence
  IsdB peptide
KAVKIVSSTH

SEQ ID NO: 230 - protein, 10 aa, Artificial Sequence
  IsdB peptide
AVKIVSSTHF

SEQ ID NO: 231 - protein, 10 aa, Artificial Sequence
  IsdB peptide
VKIVSSTHFN

SEQ ID NO: 232 - protein, 10 aa, Artificial Sequence
  IsdB peptide
KIVSSTHFNN

SEQ ID NO: 233 - protein, 10 aa, Artificial Sequence
  IsdB peptide
IVSSTHFNNK

SEQ ID NO: 234 - protein, 10 aa, Artificial Sequence
  IsdB peptide
VSSTHFNNKE

SEQ ID NO: 235 - protein, 10 aa, Artificial Sequence
  IsdB peptide
SSTHFNNKEE

SEQ ID NO: 236 - protein, 10 aa, Artificial Sequence
  IsdB peptide
STHFNNKEEK

SEQ ID NO: 237 - protein, 10 aa, Artificial Sequence
  IsdB peptide
THFNNKEEKY

SEQ ID NO: 238 - protein, 10 aa, Artificial Sequence
  IsdB peptide
HFNNKEEKYD

SEQ ID NO: 239 - protein, 10 aa, Artificial Sequence
  IsdB peptide
FNNKEEKYDY

SEQ ID NO: 240 - protein, 10 aa, Artificial Sequence
  IsdB peptide
NNKEEKYDYT

SEQ ID NO: 241 - protein, 10 aa, Artificial Sequence
  IsdB peptide
NKEEKYDYTL

SEQ ID NO: 242 - protein, 10 aa, Artificial Sequence
  IsdB peptide
KEEKYDYTLM

SEQ ID NO: 243 - protein, 10 aa, Artificial Sequence
  IsdB peptide
EEKYDYTLME

SEQ ID NO: 244 - protein, 10 aa, Artificial Sequence
  IsdB peptide
EKYDYTLMEF

SEQ ID NO: 245 - protein, 10 aa, Artificial Sequence
  IsdB peptide
KYDYTLMEFA

SEQ ID NO: 246 - protein, 10 aa, Artificial Sequence
  IsdB peptide
YDYTLMEFAQ

SEQ ID NO: 247 - protein, 10 aa, Artificial Sequence
  IsdB peptide
DYTLMEFAQP

SEQ ID NO: 248 - protein, 10 aa, Artificial Sequence
  IsdB peptide
YTLMEFAQPI

SEQ ID NO: 249 - protein, 10 aa, Artificial Sequence
  IsdB peptide
TLMEFAQPIY

SEQ ID NO: 250 - protein, 10 aa, Artificial Sequence
  IsdB peptide
LMEFAQPIYN

SEQ ID NO: 251 - protein, 10 aa, Artificial Sequence
  IsdB peptide
MEFAQPIYNS

SEQ ID NO: 252 - protein, 318 aa, Artificial Sequence
  IsdB peptide
AITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKEDDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 253 - protein, 120 aa, Artificial Sequence
  IsdB peptide
KMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTD

SEQ ID NO: 254 - protein, 60 aa, Artificial Sequence
  IsdB peptide
KMTDLQDTKYVVYESVENNESMMDTFVKHPIKTGMLNGKKYMVMETTNDDYWKDFMVEGQ

SEQ ID NO: 255 - protein, 60 aa, Artificial Sequence
  IsdB peptide
RVRTISKDAKNNTRTIIFPYVEGKTLYDAIVKVHVKTIDYDGQYHVRIVDKEAFTKANTD

SEQ ID NO: 256 - protein, 20 aa, Artificial Sequence
  IsdB peptide
KMTDLQDTKYVVYESVENNE

SEQ ID NO: 257 - protein, 20 aa, Artificial Sequence
  IsdB peptide
SMMDTFVKHPIKTGMLNGKK

SEQ ID NO: 258 - protein, 20 aa, Artificial Sequence
  IsdB peptide
YMVMETTNDDYWKDFMVEGQ

SEQ ID NO: 259 - protein, 20 aa, Artificial Sequence
  IsdB peptide
VVYESVENNESMMDTFVKHP

SEQ ID NO: 260 - protein, 20 aa, Artificial Sequence
  IsdB peptide
IKTGMLNGKKYMVMETTNDD

SEQ ID NO: 261 - protein, 20 aa, Artificial Sequence
  IsdB peptide
RVRTISKDAKNNTRTIIFPY

SEQ ID NO: 262 - protein, 20 aa, Artificial Sequence
  IsdB peptide
VEGKTLYDAIVKVHVKTIDY

SEQ ID NO: 263 - protein, 20 aa, Artificial Sequence
  IsdB peptide
DGQYHVRIVDKEAFTKANTD

SEQ ID NO: 264 - protein, 20 aa, Artificial Sequence
  IsdB peptide
NNTRTIIFPYVEGKTLYDAI

SEQ ID NO: 265 - protein, 20 aa, Artificial Sequence
  IsdB peptide
VKVHVKTIDYDGQYHVRIVD

SEQ ID NO: 266 - protein, 10 aa, Artificial Sequence
  IsdB peptide
KMTDLQDTKY

SEQ ID NO: 267 - protein, 10 aa, Artificial Sequence
  IsdB peptide
MTDLQDTKYV

SEQ ID NO: 268 - protein, 10 aa, Artificial Sequence
  IsdB peptide
TDLQDTKYVV

SEQ ID NO: 269 - protein, 10 aa, Artificial Sequence
  IsdB peptide
DLQDTKYVVY

SEQ ID NO: 270 - protein, 10 aa, Artificial Sequence
  IsdB peptide
LQDTKYVVYE

SEQ ID NO: 271 - protein, 10 aa, Artificial Sequence
  IsdB peptide
QDTKYVVYES

SEQ ID NO: 272 - protein, 10 aa, Artificial Sequence
  IsdB peptide
DTKYVVYESV

SEQ ID NO: 273 - protein, 10 aa, Artificial Sequence
  IsdB peptide
TKYVVYESVE

SEQ ID NO: 274 - protein, 10 aa, Artificial Sequence
  IsdB peptide
KYVVYESVEN

SEQ ID NO: 275 - protein, 10 aa, Artificial Sequence
  IsdB peptide
YVVYESVENN

SEQ ID NO: 276 - protein, 10 aa, Artificial Sequence
  IsdB peptide
VVYESVENNE

SEQ ID NO: 277 - protein, 10 aa, Artificial Sequence
  IsdB peptide
VYESVENNES

SEQ ID NO: 278 - protein, 10 aa, Artificial Sequence
  IsdB peptide
YESVENNESM

SEQ ID NO: 279 - protein, 10 aa, Artificial Sequence
  IsdB peptide
ESVENNESMM

SEQ ID NO: 280 - protein, 10 aa, Artificial Sequence
  IsdB peptide
SVENNESMMD

SEQ ID NO: 281 - protein, 10 aa, Artificial Sequence
  IsdB peptide
VENNESMMDT

SEQ ID NO: 282 - protein, 10 aa, Artificial Sequence
  IsdB peptide
ENNESMMDTF

SEQ ID NO: 283 - protein, 10 aa, Artificial Sequence
  IsdB peptide
NNESMMDTFV

SEQ ID NO: 284 - protein, 10 aa, Artificial Sequence
  IsdB peptide
NESMMDTFVK

SEQ ID NO: 285 - protein, 10 aa, Artificial Sequence
  IsdB peptide
ESMMDTFVKH

SEQ ID NO: 286 - protein, 10 aa, Artificial Sequence
  IsdB peptide
SMMDTFVKHP

SEQ ID NO: 287 - protein, 10 aa, Artificial Sequence
  IsdB peptide
MMDTFVKHPI

SEQ ID NO: 288 - protein, 10 aa, Artificial Sequence
  IsdB peptide
MDTFVKHPIK

SEQ ID NO: 289 - protein, 10 aa, Artificial Sequence
  IsdB peptide
DTFVKHPIKT

SEQ ID NO: 290 - protein, 10 aa, Artificial Sequence
  IsdB peptide
TFVKHPIKTG

SEQ ID NO: 291 - protein, 10 aa, Artificial Sequence
  IsdB peptide
FVKHPIKTGM

SEQ ID NO: 292 - protein, 10 aa, Artificial Sequence
  IsdB peptide
VKHPIKTGML

SEQ ID NO: 293 - protein, 10 aa, Artificial Sequence
  IsdB peptide
KHPIKTGMLN

SEQ ID NO: 294 - protein, 10 aa, Artificial Sequence
  IsdB peptide
HPIKTGMLNG

SEQ ID NO: 295 - protein, 10 aa, Artificial Sequence
  IsdB peptide
PIKTGMLNGK

SEQ ID NO: 296 - protein, 10 aa, Artificial Sequence
  IsdB peptide
IKTGMLNGKK

SEQ ID NO: 297 - protein, 10 aa, Artificial Sequence
  IsdB peptide
KTGMLNGKKY

SEQ ID NO: 298 - protein, 10 aa, Artificial Sequence
  IsdB peptide
TGMLNGKKYM

SEQ ID NO: 299 - protein, 10 aa, Artificial Sequence
  IsdB peptide
GMLNGKKYMV

SEQ ID NO: 300 - protein, 10 aa, Artificial Sequence
  IsdB peptide
MLNGKKYMVM

SEQ ID NO: 301 - protein, 10 aa, Artificial Sequence
  IsdB peptide
LNGKKYMVME

SEQ ID NO: 302 - protein, 10 aa, Artificial Sequence
  IsdB peptide
NGKKYMVMET

SEQ ID NO: 303 - protein, 10 aa, Artificial Sequence
  IsdB peptide
GKKYMVMETT

SEQ ID NO: 304 - protein, 10 aa, Artificial Sequence
  IsdB peptide
KKYMVMETTN

SEQ ID NO: 305 - protein, 10 aa, Artificial Sequence
  IsdB peptide
KYMVMETTND

SEQ ID NO: 306 - protein, 10 aa, Artificial Sequence
  IsdB peptide
YMVMETTNDD

SEQ ID NO: 307 - protein, 10 aa, Artificial Sequence
  IsdB peptide
MVMETTNDDY

SEQ ID NO: 308 - protein, 10 aa, Artificial Sequence
  IsdB peptide
DFMVEGQRVR

SEQ ID NO: 309 - protein, 10 aa, Artificial Sequence
  IsdB peptide
FMVEGQRVRT

SEQ ID NO: 310 - protein, 10 aa, Artificial Sequence
  IsdB peptide
MVEGQRVRTI

SEQ ID NO: 311 - protein, 10 aa, Artificial Sequence
  IsdB peptide
VEGQRVRTIS

SEQ ID NO: 312 - protein, 10 aa, Artificial Sequence
  IsdB peptide
EGQRVRTISK

SEQ ID NO: 313 - protein, 10 aa, Artificial Sequence
  IsdB peptide
GQRVRTISKD

SEQ ID NO: 314 - protein, 10 aa, Artificial Sequence
  IsdB peptide
QRVRTISKDA

SEQ ID NO: 315 - protein, 9 aa, Artificial Sequence
  IsdB peptide
VRTISKDAK

SEQ ID NO: 316 - protein, 10 aa, Artificial Sequence
  IsdB peptide
VRTISKDAKN

SEQ ID NO: 317 - protein, 10 aa, Artificial Sequence
  IsdB peptide
TRTIIFPYVE

SEQ ID NO: 318 - protein, 10 aa, Artificial Sequence
  IsdB peptide
RTIIFPYVEG

SEQ ID NO: 319 - protein, 10 aa, Artificial Sequence
  IsdB peptide
TIIFPYVEGK

SEQ ID NO: 320 - protein, 10 aa, Artificial Sequence
  IsdB peptide
IIFPYVEGKT

SEQ ID NO: 321 - protein, 10 aa, Artificial Sequence
  IsdB peptide
IFPYVEGKTL

SEQ ID NO: 322 - protein, 10 aa, Artificial Sequence
  IsdB peptide
FPYVEGKTLY

SEQ ID NO: 323 - protein, 10 aa, Artificial Sequence
  IsdB peptide
PYVEGKTLYD

SEQ ID NO: 324 - protein, 10 aa, Artificial Sequence
  IsdB peptide
YVEGKTLYDA

SEQ ID NO: 325 - protein, 10 aa, Artificial Sequence
  IsdB peptide
VEGKTLYDAI

SEQ ID NO: 326 - protein, 10 aa, Artificial Sequence
  IsdB peptide
EGKTLYDAIV

SEQ ID NO: 327 - protein, 10 aa, Artificial Sequence
  IsdB peptide
GKTLYDAIVK

SEQ ID NO: 328 - protein, 10 aa, Artificial Sequence
  IsdB peptide
TLYDAIVKVH

SEQ ID NO: 329 - protein, 10 aa, Artificial Sequence
  IsdB peptide
LYDAIVKVHV

SEQ ID NO: 330 - protein, 10 aa, Artificial Sequence
  IsdB peptide
YDAIVKVHVK

SEQ ID NO: 331 - protein, 10 aa, Artificial Sequence
  IsdB peptide
DAIVKVHVKT

SEQ ID NO: 332 - protein, 10 aa, Artificial Sequence
  IsdB peptide
AIVKVHVKTI

SEQ ID NO: 333 - protein, 10 aa, Artificial Sequence
  IsdB peptide
IVKVHVKTID

SEQ ID NO: 334 - protein, 10 aa, Artificial Sequence
  IsdB peptide
VKVHVKTIDY

SEQ ID NO: 335 - protein, 10 aa, Artificial Sequence
  IsdB peptide
KVHVKTIDYD

SEQ ID NO: 336 - protein, 10 aa, Artificial Sequence
  IsdB peptide
VHVKTIDYDG

SEQ ID NO: 337 - protein, 10 aa, Artificial Sequence
  IsdB peptide
HVKTIDYDGQ

SEQ ID NO: 338 - protein, 10 aa, Artificial Sequence
  IsdB peptide
VKTIDYDGQY

SEQ ID NO: 339 - protein, 10 aa, Artificial Sequence
  IsdB peptide
KTIDYDGQYH

SEQ ID NO: 340 - protein, 10 aa, Artificial Sequence
  IsdB peptide
TIDYDGQYHV

SEQ ID NO: 341 - protein, 10 aa, Artificial Sequence
  IsdB peptide
IDYDGQYHVR

SEQ ID NO: 342 - protein, 10 aa, Artificial Sequence
  IsdB peptide
DYDGQYHVRI

SEQ ID NO: 343 - protein, 10 aa, Artificial Sequence
  IsdB peptide
YDGQYHVRIV

SEQ ID NO: 344 - protein, 10 aa, Artificial Sequence
  IsdB peptide
DGQYHVRIVD

SEQ ID NO: 345 - protein, 10 aa, Artificial Sequence
  IsdB peptide
GQYHVRIVDK

SEQ ID NO: 346 - protein, 10 aa, Artificial Sequence
  IsdB peptide
QYHVRIVDKE

SEQ ID NO: 347 - protein, 10 aa, Artificial Sequence
  IsdB peptide
YHVRIVDKEA

SEQ ID NO: 348 - protein, 10 aa, Artificial Sequence
  IsdB peptide
HVRIVDKEAF

SEQ ID NO: 349 - protein, 10 aa, Artificial Sequence
  IsdB peptide
VRIVDKEAFT

SEQ ID NO: 350 - protein, 10 aa, Artificial Sequence
  IsdB peptide
RIVDKEAFTK

SEQ ID NO: 351 - protein, 10 aa, Artificial Sequence
  IsdB peptide
IVDKEAFTKA

SEQ ID NO: 352 - protein, 10 aa, Artificial Sequence
  IsdB peptide
VDKEAFTKAN

SEQ ID NO: 353 - protein, 10 aa, Artificial Sequence
  IsdB peptide
DKEAFTKANT

SEQ ID NO: 354 - protein, 10 aa, Artificial Sequence
  IsdB peptide
KEAFTKANTD

SEQ ID NO: 355 - DNA, 26 bp, Artificial Sequence
  Synthetic primer
aactcgaggcagctgaagaaacaggt

SEQ ID NO: 356 - DNA, 26 bp, Artificial Sequence
  Synthetic primer
aaggatcccacttgctcatctaaagc

SEQ ID NO: 357 - DNA, 24 bp, Artificial Sequence
  Synthetic primer
aactcgaggcmagatgagcaagtg

SEQ ID NO: 358 - DNA, 26 bp, Artificial Sequence
  Synthetic primer
aaggatcctgattttgctttattttc

SEQ ID NO: 359 - protein, 112 aa, Artificial Sequence
  Synthetic peptide
DVVMTQTPLSLPVSLGDQASISCRSSQSLVYSNGNTYLHWFLQKPGQSPKLLIYKVSNRFSGVPDRFSGSGSGTDFTLKISRVEAEDLGVYFCSQTTHIPLTFGAGTKLELK

SEQ ID NO: 360 - DNA, 337 bp, Artificial Sequence
  Synthetic primer
gatgttgtgatgacccagactccactctccctgcctgtcagtcttggagatcaagcctccatctcttgcagatctagtcagagccttgtatatagtaatggaaacacctatttacattggttcctgcagaagccaggccagtctccaaagctcctgatctacaaagtttccaaccgattttctggggtcccagacaggttcagtggcagtggatcagggacagatttcacactcaagatctccagagtggaggctgaggatctgggagtttatttctgctctcaaactacacatattccgctcacgttcggtgctgggaccaagctggagctgaaac

SEQ ID NO: 361 - protein, 118 aa, Artificial Sequence
  Synthetic peptide
EVQLLQSGPELVKPGTSVKMSCRTSGYTFTEYTMHWVKQSHEKRLEWIGGIDPSNGDTSYNQKFKGKATLTVDKSSSSAYMDLRSLTSVDSAIYYCARLEGVLPLDYWGHGTTLTVSS

SEQ ID NO: 362 - DNA, 355 bp, Artificial Sequence
  Synthetic primer
gaggtccagctgctacagtctggacctgaactggtgaagcctgggacttcagtgaagatgtcctgcaggacttctggatacacattcactgaatacaccatgcactgggtgaagcagagccatgaaaagagacttgagtggattggaggtattgatcctagcaatggtgatactagctacaaccagaagttcaagggcaaggccacattgactgtagacaagtcctccagctcagcctacatggacctccgcagcctgacatctgtggattctgcaatctattactgtgcaagactggaaggagtactaccccttgactactggggccacggcaccactctcacagtctcctcag

SEQ ID NO: 363 - protein, 112 aa, Artificial Sequence; identical to SEQ ID NO: 359
  Synthetic peptide

SEQ ID NO: 364 - DNA, 337 bp, Artificial Sequence
  Synthetic primer
gatgttgtgatgacccaaactccactctccctgcctgtcagtcttggagatcaagcctccatctcttgcagatctagtcagagccttgtatatagtaatggaaacacctatttacattggttcctgcagaagccaggccagtctccaaagctcctgatctacaaagtttccaaccgattttctggggtcccagacaggttcagtggcagtggatcagggacagatttcacactcaagatctccagagtggaggctgaggatctgggagtttatttctgctctcaaactacacatattccgctcacgttcggtgctgggaccaagctggagctgaaac

SEQ ID NO: 365 - protein, 113 aa, Artificial Sequence
  Synthetic peptide
XSXLXXXXXSLPVSLGDQASISXRSSQSLVHSNGNTYLHWFLXKPGQSPKLLIYKVSNRFSGVPGRFSGSGSGTDFTLKISRVEAEDLGVYFCSQSTHVPPLTFGAGTKLELK

SEQ ID NO: 366 - DNA, 340 bp, Artificial Sequence
  Synthetic primer
tgntctgncctcncntnanctcntntatccctgcctgtcagtcttggagatcaagcctccatctctngcagatctagtcagagccttgtacacagtaatggaaacacctatttacattggttcctgcanaagccaggccagtctccaaagctcctgatctacaaagtttccaaccgattttcnggggtcccaggcaggttcagtggcagtggatcagggacagatttcacactcaagatcagcagagtggaggctgaggatctgggagtttatttctgttctcaaagtacacatgttcctccgctcacgttcggtgctgggaccaagctggagctgaagc

SEQ ID NO: 367 - protein, 112 aa, Artificial Sequence
  Synthetic peptide
XXXXTQTPLSLPVSLGDQASISCSSSQNIVHSNGYTYLEWYLQKPGQSPKLLIYKVSNRFSGVPDRFSGSGSGTDFTLKISRVEAEDLGVYFCFQGSHVPYTFGGGTKLEIK

SEQ ID NO: 368 - DNA, 337 bp, Artificial Sequence
  Synthetic primer
tgnngnttnntgacccaaactccactctccctgcctgtcagtcttggagatcaagcctccatctcttgcagctctagtcagaacattgttcatagtaatggatacacctatttagaatggtacctgcagaaaccaggccagtctccaaagctcctgatctacaaagtttccaaccgattttctggggtcccagacaggttcagtggcagtggttcagggacagatttcacactcaagatcagcagagtggaggctgaggatctgggagtttatttctgctttcaaggttcacatgttccgtacacgttcggaggggggaccaagctggaaataaaac

SEQ ID NO: 369 - protein, 113 aa, Artificial Sequence
  Synthetic peptide
DVQLQESGPGLVKPSQSLSLTCTVTGYSITSDYAWNWIRQFPGNKLEWLGSIIFTGATDYNPSLKSXISITRDTSKNQFFLHLTXMTTEDTATYYCTRELRGWGQGTTLTVSS

SEQ ID NO: 370 - DNA, 340 bp, Artificial Sequence
  Synthetic primer
gatgtgcagcttcaggagtcgggacctggcctggtgaagccttctcagtctctgtccctcacctgcactgtcactggctactcaatcaccagtgattatgcctggaactggatccggcagtttccaggaaacaaactggagtggttgggctccataatcttcactggtgccactgactacaacccatctctcaaaagtngaatctctatcactcgagacacatccaagaaccagttcttcctgcacttgacttntatgactactgaggacacagccacatattattgtacaagagaacttagaggctggggccaaggcaccactctcacagtctcctcag

SEQ ID NO: 371 - protein, 112 aa, Artificial Sequence
  Synthetic peptide
DVVMTQTPLSLPVSLGDQASISCRSSQSLLYSNGNTYLHWYLQKPGQSPKLLIYKVSNRFSGVPDRFSASGSGTDFTLKISRVEAEDLGVYFCSQSTHVPLTFGAGTKLELK

SEQ ID NO: 372 - DNA, 337 bp, Artificial Sequence
  Synthetic primer
gatgttgtgatgacccaaactccactctccctgcctgtcagtcttggagatcaagcctccatctcttgcagatctagtcagagcctattatacagtaatggaaacacctatttacattggtacctgcagaagccaggccagtctccaaagctcctgatctacaaagtttccaaccgattttctggggtcccagacaggttcagtgccagtggatcagggacagatttcacactcaagatcagcagagtggaggctgaggatctgggagtttatttctgttctcaaagtacacatgttccgctcacgttcggtgctgggaccaagctggagctgaaac

SEQ ID NO: 373 - protein, 118 aa, Artificial Sequence
  Synthetic peptide
EVQLQESGPELVKPGTSVWISCKTSGFTFTKYTMHWVKQSHGKTLEWIGGIDPNNGDTSYNQKFKDKATLTVDKSSSTAYMELRSLTSEDSAVFFCVRLEGSLPLDYWGQGTTLTVSS

SEQ ID NO: 374 - DNA, 355 bp, Artificial Sequence
  Synthetic primer
gaggtccagctgcaagagtctggacctgaactggtgaagcctgggacttcagtgtggatatcctgcaagacttctggattcacattcactaaatacaccatgcactgggtgaagcagagccatggaaagacccttgagtggattggaggtattgatcctaacaatggtgatactagttacaaccagaagttcaaggacaaggccacattgactgtagacaagtcctccagcacagcctacatggaactccgcagcctgacatctgaagattctgcagtctttttctgtgtaagactggaagggtcactgccccttgactactggggccaaggcaccactctcacagtctcctcag

SEQ ID NO: 375 - protein, 110 aa, Artificial Sequence
  Synthetic peptide
GLTGEPGTSVKMSCRTSGYTFTEYTMHWVKQSHEKRLEWIGGIDPSNGDTSYNQKFKGKATLTVDKSSSSAYMDLRSLTSVDSAIYYCARLEGVLPLDYWGHGTTLTVSS

SEQ ID NO: 376 - DNA, 331 bp, Artificial Sequence
  Synthetic primer
ggactgactggtgagcctgggacttcagtgaagatgtcctgcaggacttctggatacacattcactgaatacaccatgcactgggtgaagcagagccatgaaaagagacttgagtggattggaggtattgatcctagcaatggtgatactagctacaaccagaagttcaagggcaaggccacattgactgtagacaagtcctccagctcagcctacatggacctccgcagcctgacatctgtggattctgcaatctattactgtgcaagactggaaggagtactaccccttgactactggggccacggcaccactctcacagtctcctcag

SEQ ID NO: 377 - protein, 107 aa, Artificial Sequence
  Synthetic peptide
DIVMTQSQKFMSTSVRDRVAVTCKASQNVGTNVAWYQQKPGQSPKALIYSASYRYSGVPDRFTGSGSGTDFTLTISNVQSEDLAEYFCQQYNSYPYTFGGGTKLEVK

SEQ ID NO: 378 - DNA, 322 bp, Artificial Sequence
  Synthetic primer
gacattgtgatgacccagtctcaaaaattcatgtccacatcagtaagagacagggtcgccgtcacctgcaaggccagtcagaatgtgggtactaatgtagcctggtatcaacagaaaccaggtcaatctcctaaagcactgatttactcggcatcctaccggtacagtggagtccctgatcgcttcacaggcagtggatctgggacagatttcactctcaccatcagcaatgtgcagtctgaagacttggcagagtatttctgtcagcagtataacagctatccgtacacgttcggaggggggaccaagctggaagtaaaac

SEQ ID NO: 379 - protein, 118 aa, Artificial Sequence; identical to SEQ ID NO: 373
  Synthetic peptide

SEQ ID NO: 380 - DNA, 355 bp, Artificial Sequence; identical to SEQ ID NO: 374
  Synthetic primer

SEQ ID NO: 381 - protein, 112 aa, Artificial Sequence
  Synthetic peptide
SLDLTGEPGASVKMSCRTSGYTFTEYTMHWVKQSHEKSLEWIGGIDPDNGDTSFNQKFKGKATLTVDKSSSTAYMELRSLTYDDTAIYLCARLEGVLPLDYWGQGTTLTVSS

SEQ ID NO: 382 - DNA, 337 bp, Artificial Sequence
  Synthetic primer
agtctggacctgactggtgagcctggggcttcagtgaagatgtcctgcaggacttctggatacacattcactgaatacaccatgcactgggtgaagcagagccatgaaaagagccttgaatggattggaggtattgatcctgacaatggtgatactagcttcaaccagaagttcaagggcaaggccacattgactgtagacaagtcctccagcacagcctacatggagctccgcagcctgacatatgacgatactgcaatctatctctgtgcaagactggaaggagtactcccccttgactactggggccaaggcaccactctcacagtctcctcag

SEQ ID NO: 383 - protein, 107 aa, Artificial Sequence; identical to SEQ ID NO: 377
  Synthetic peptide

SEQ ID NO: 384 - DNA, 322 bp, Artificial Sequence; identical to SEQ ID NO: 378
  Synthetic primer

SEQ ID NO: 385 - protein, 116 aa, Artificial Sequence
  Synthetic peptide
QVQLQQSGAELVRPGTSVKVSCKASGNAFTNYLIEWIKQRPGQGLEWIGVINPGSGITNYNEKFKGKATLTADKSSNTAYMQLSSLSSDDSAVYFCSGSANWFAYWGQGTLVTVSA

SEQ ID NO: 386 - DNA, 348 bp, Artificial Sequence
  Synthetic primer
caggtccagctgcagcagtctggagctgaactggtaaggcctgggacttcagtgaaggtgtcctgcaaggcttctggaaacgccttcactaattatttaatagagtggataaaacagaggcctggacagggccttgagtggattggagtgattaatcctggaagtggaattactaactacaatgagaagttcaagggcaaggcaacactgactgcagacaaatcctccaacactgcctacatgcagctcagcagcctgtcatctgatgactctgcggtctatttctgttcaggatcggccaactggtttgcttactggggccaagggactctggtcactgtctctgca

SEQ ID NO: 387 - protein, 113 aa, Artificial Sequence
  Synthetic peptide
DVLMTQTPLSLPVSLGDQASISCSSSQNIVHSNGYTYLEWYLQKPGQSPKLLIYKVSNRFSGVPDRFSGSGSGTDFTLKISRVEAEDLGVYFCFQGSHVPYTFGGGTKLEIKR

SEQ ID NO: 388 - DNA, 338 bp, Artificial Sequence
  Synthetic primer
gatgttttgatgacccaaactccactctccctgcctgtcagtcttggagatcaagcctccatctcttgcagctctagtcagaacattgttcatagtaatggatacacctatttagaatggtacctgcagaaaccaggccagtctccaaagctcctgatctacaaagtttccaaccgattttctggggtcccagacaggttcagtggcagtggttcagggacagatttcacactcaagatcagcagagtggaggctgaggatctgggagtttatttctgctttcaaggttcacatgttccgtacacgttcggaggggggaccaagctggaaataaaacg

SEQ ID NO: 389 - protein, 117 aa, Artificial Sequence
  Synthetic peptide
EVQLVESGGGLVQPGGSLKLSCAASGFTFGSYGMSWVRQTPDKRLELVAIINRNGGSTDYPDSVKGRFTISRDNAKNTLYLQMSSLKSEDTAMYNCVREGYGHFDHWGQGTTLTVSS

SEQ ID NO: 390 - DNA, 351 bp, Artificial Sequence
  Synthetic primer
gaggtgcagctggtggagtcggggggaggcttagtgcagcctggagggtccctgaaactctcctgtgcagcctctggattcactttcggtagctatggcatgtcttgggttcgccagactccagacaagaggctggagttggtcgcaatcattaatagaaatggtggtagcaccgattatccagacagtgtgaagggccgattcaccatctccagagacaatgccaagaacaccctgtacctgcaaatgagcagtctgaagtctgaggacacagccatgtataactgtgtaagagagggttatggtcactttgaccactggggccaaggcaccactctcacagtctcctca

SEQ ID NO: 391 - protein, 118 aa, Artificial Sequence
  Synthetic peptide
SDVQLQESGPGLVKPSQSLSLTCSVTGHSITSGYYWNWIRQFPGNKLEWMGYISFDGRNKYNPSLKNRISITRDTSKNQFFLKLNSVTSEDTATYFCTRLSYSTLDYWGQGTSVTVSS

SEQ ID NO: 392 - DNA, 354 bp, Artificial Sequence
  Synthetic primer
tctgatgtacagcttcaggagtcaggacctggcctcgtgaaaccttctcagtctctgtctctcacctgctctgtcactggccactccatcaccagtggttattactggaactggatccggcagtttccaggaaacaaactggaatggatgggctacataagtttcgacggtcgcaataagtacaacccatctctcaaaaatcgaatctccatcactcgtgacacatctaagaaccagtttttcctgaagttgaattctgtgacctctgaggacacagctacatatttctgtacaagactaagttactctactctggactactggggtcaaggaacctcagtcaccgtctcctca

SEQ ID NO: 393 - protein, 113 aa, Artificial Sequence
  Synthetic peptide
DVVMTQTPLSLPVSLGDQASISCRSSQSLVHSNGNTYLHWYLQKPGQSPKLLIYKVSSRFSGVPDRFSGSGSGTDFTLKISRVEAEDLGVYFCSQSTHVPYTFGGGTKLEIKR

SEQ ID NO: 394 - DNA, 338 bp, Artificial Sequence
  Synthetic primer
gatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccagccgattttctggggtcccagacaggttcagtggcagtggatcagggacagatttcacactcaagatcagcagagtggaggctgaggatctgggagtttatttctgctctcaaagtacacatgttccgtacacgttcggaggggggaccaagctggaaataaaacg

SEQ ID NO: 395 - protein, 117 aa, Artificial Sequence
  Synthetic peptide
EVQLVESGGGLVKPGGSLKISCAASGFTFSDYSMYWVRQTPEKRLEWVATISEGGSYINYPDNVKGRFTISRDNAKNNLYLQMSSLKSEDAAMYYCARDYDYDAFAYWGQGTLVTVS

SEQ ID NO: 396 - DNA, 352 bp, Artificial Sequence
  Synthetic primer
gaagtgcagctggtggagtctgggggaggcttagtgaagcctggagggtccctgaaaatctcctgtgcagcctctggattcactttcagtgactattccatgtattgggttcgccagactccggaaaagaggctggagtgggtcgcaaccattagtgaaggtggtagttacatcaactatccagacaatgtgaaggggcgattcaccatctccagagacaatgccaagaacaacctgtacctgcaaatgagcagtctgaagtctgaggacgcagccatgtattactgtgcaagagactatgattacgacgcttttgcttactggggccaagggactctggtcactgtctctg

SEQ ID NO: 397 - protein, 112 aa, Artificial Sequence
  Synthetic peptide
DVGMTQTPLSLPVSLGDQASISCGSSQSLLHSNGKTYLHWYLQKPGQSPKLLIYKVSNRFSGVPDRFSGSGSGTYFTLKITRVEAEDLGVYFCSQTTHVPFTFGSGTKLEIK

SEQ ID NO: 398 - DNA, 337 bp, Artificial Sequence
  Synthetic primer
gatgttgggatgacccaaactcctctctccctgcctgtcagtcttggagatcaagcctccatctcttgcggatctagtcagagccttctacacagtaatggaaagacctatttacactggtacctgcagaagccaggccagtctccaaagctcctgatctacaaagtttccaaccgattttctggggtccccgacaggttcagtggcagtggatcagggacatatttcacactcaagatcaccagagtggaggctgaggatctgggagtttatttctgctctcaaactacccatgttccattcacgttcggctcggggacaaagttggaaataaaac

(19) United States
(12) Patent Application Publication
(10) Pub. No.: US 2014/0037650 A1
(43) Pub. Date: Feb. 6, 2014

(54) COMPOSITIONS AND METHODS RELATED TO ANTIBODIES TO STAPHYLOCOCCAL PROTEINS ISDA OR ISDB

(71) Applicant: University of Chicago, Chicago, IL (US)

(72) Inventors: Hwan Keun Kim, Naperville, IL (US); Andrea DeDent, Chicago, IL (US); Carla Emolo, Chicago, IL (US); Dominique M. Missaikas, Chicago, IL (US); Olaf Schneewind, Chicago, IL (US)

(73) Assignee: University of Chicago, Chicago, IL (US)

(21) Appl. No.: 13/959,147

(22) Filed: Aug. 5, 2013

Related U.S. Application Data
(63) Continuation of application No. PCT/US2012/028618, filed on Mar. 9, 2012.
Application No. PCT/US2012/028618 is a continuation-in-part of application No. 12/842,811, filed on Jul. 23, 2010, now abandoned.
(60) Provisional application No. 61/451,471, filed on Mar. 10, 2011.
(60) Provisional application No. 61/526,166, filed on Aug. 22, 2011.
(60) Provisional application No. 61/228,479, filed on Jul. 24, 2009.

(51) Int. Cl. C07K 16/12; A61K 45/06; A61K 39/40

(52) U.S. Cl. CPC C07K 16/1271; A61K 39/40; A61K 45/06
USPC 424/165.1; 530/389.5; 530/388.4; 530/387.3

(57) ABSTRACT

The present invention concerns methods and compositions for treating or preventing a bacterial infection, particularly infection by a Staphylococcus bacterium. The invention provides methods and compositions for providing a passive immune response against the bacteria. In certain embodiments, the methods and compositions involve an antibody, such as a recombinant antibody, that binds IsdA and/or IsdB polypeptides.

This application claims the benefit of U.S. Provisional Patent Application Nos. 61/451,471, filed on Mar. 10, 2011 and 61/526,166, filed Aug. 22, 2011, the entirety of which are incorporated herein by reference.

This invention was made with government support under AI52747, AI92711 and 1-U54-AI-057153 from the National Institutes of Health. The government has certain rights in the invention.

### I. FIELD OF THE INVENTION

The present invention relates generally to the fields of immunology, microbiology, and pathology. More particularly, it concerns methods and compositions involving antibodies to bacterial proteins and bacterial peptides used to elicit such antibodies. The proteins include IsdA and IsdB proteins and peptides.

### II. BACKGROUND

The number of both community acquired and hospital acquired infections have increased over recent years with the increased use of intravascular devices. Hospital acquired (nosocomial) infections are a major cause of morbidity and mortality, more particularly in the United States, where they affect more than 2 million patients annually. The most frequent nosocomial infections are urinary tract infections (33% of the infections), followed by pneumonia (15.5%), surgical site infections (14.8%) and primary bloodstream infections (13%) (Emorl and Gaynes, 1993).

Staphylococcus aureus, Coagulase-negative Staphylococci (mostly Staphylococcus epidermidis), enterococcus spp., Escherichia coli and Pseudomonas aeruginosa are the major nosocomial pathogens. Although these pathogens almost cause the same number of infections, the severity of the disorders they can produce combined with the frequency of antibiotic resistant isolates balance this ranking towards S. aureus and S. epidermidis as being the most significant nosocomial pathogens.

Staphylococcus can cause a wide variety of diseases in humans and other animals through either toxin production or invasion. Staphylococcal toxins are a common cause of food poisoning, as the bacteria can grow in improperly-stored food.

Staphylococcus epidermidis is a normal skin commensal, which is also an important opportunistic pathogen responsible for infections of impaired medical devices and infections at sites of surgery. Medical devices infected by S. epidermidis include cardiac pacemakers, cerebrospinal fluid shunts, continuous ambulatory peritoneal dialysis catheters, orthopedic devices and prosthetic heart valves.

Staphylococcus aureus is the most common cause of nosocomial infections with a significant morbidity and mortality. It is the cause of some cases of osteomyelitis, endocarditis, septic arthritis, pneumonia, abscesses and toxic shock syndrome.

S. aureus can survive on dry surfaces, increasing the chance of transmission. Any S. aureus infection can cause the staphylococcal scalded skin syndrome, a cutaneous reaction to exotoxin absorbed into the bloodstream. S. aureus can also cause a type of septicemia called pyaemia that can be life-threatening. Methicillin-resistant Staphylococcus aureus (MRSA) has become a major cause of hospital-acquired infections.

S. aureus and S. epidermidis infections are typically treated with antibiotics, with penicillin being the drug of choice, but vancomycin being used for methicillin resistant isolates. The percentage of staphylococcal strains exhibiting wide-spectrum resistance to antibiotics has increased, posing a threat to effective antimicrobial therapy. In addition, the recent appearance of vancomycin-resistant S. aureus strain has aroused fear that MRSA strains for which no effective therapy is available are starting to emerge and spread.

An alternative approach to antibiotics in the treatment of staphylococcal infections has been the use of antibodies against staphylococcal antigens in passive immunotherapy. Examples of this passive immunotherapy involves administration of polyclonal antisera (WO00/15238, WO00/12132) as well as treatment with monoclonal antibodies against lipoteichoic acid (WO98/57994).

The first generation of vaccines targeted against S. aureus or against the exoproteins it produces have met with limited success (Lee, 1996) and there remains a need to develop additional therapeutic compositions for treatment of staphylococcus infections.

### SUMMARY OF THE INVENTION

Staphylococcus aureus is the most frequent cause of bacteremia and hospital-acquired infection in the United States. An FDA approved vaccine that prevents staphylococcal disease is currently unavailable. Two sortase-anchored surface proteins, IsdA and IsdB, have been identified as subunit vaccines that, following active immunization, protect experimental animals against intravenous challenge with staphylococci. The inventors have identified the molecular basis of this immunity and report that, when passively transferred to naive mice, purified antibodies directed against IsdA or IsdB protect against staphylococcal abscess formation and lethal intravenous challenge. When added to mouse blood, IsdA or IsdB specific antibodies do not promote opsonophagocytosis of wild-type staphylococci. However, antibodies directed against IsdB interfere with the ability of this surface protein to bind hemoglobin or heme. As the structural genes for isdA and isdB are required for heme-iron scavenging during the pathogenesis of infection, IsdA and IsdB antibodies likely provide protection against staphylococci by blocking the pathogen's heme-iron scavenging mechanisms.

In certain embodiments the invention provides an antibody composition that inhibits, ameliorates, and/or prevents Staphylococcal infection.

Certain embodiments are directed to a recombinant peptide comprising 1, 2, 3, 4, 5, 6, 7, 8, 9, 10 or more amino acid segments comprising about, at least or at most 5, 6, 7, 8, 9, 10, 11, 12, 13, 14, 15, 16, 17, 18, 19, 20, 21, 22, 23, 24, 25 to 25, 26, 27, 28, 29, 30, 31, 32, 33, 34, 35, 36, 37, 38, 39, 40, 41, 42, 43, 44, 45, 46, 47, 48, 49, 50 amino acids in length, including all values and ranges there between, that are at least 80, 85, 90, 95, 96, 97, 98, 99, or 100% identical to amino acid segments of Staphylococcal IsdA or IsdB protein (SEQ ID NO:1 or 2, respectively). In further aspects, the invention is directed to antibodies that specifically bind one or more of these particular amino acid segments.

A polypeptide can comprise at least three amino acid segments that are at least 90% identical to an amino acid segment of Staphylococcal IsdA or IsdB protein. In certain aspects the amino acid segments are identical to each other. In other aspects the amino acid segments are heterogenous or homogenous. The polypeptide can further comprise a non-staphylococcal peptide, such as an adjuvant, label, or tag.

The present invention also provides for the use of IsdA and/or IsdB antibodies in methods and compositions for the treatment of bacterial and/or staphylococcal infection. In certain embodiments, the compositions of the invention are used in the manufacture of medicaments for the therapeutic and/or prophylactic treatment of bacterial infections, particularly staphylococcus infections. Furthermore, the present invention provides methods and compositions that can be used to treat (e.g., limiting staphylococcal abscess formation and/or persistence in a subject) or prevent bacterial infection.

Certain aspects are directed to methods of reducing Staphylococcus infection or abscess formation comprising administering to a patient having or suspected of having a Staphylococcus infection an effective amount of one or more purified antibodies that specifically bind a Staphylococcal IsdA, IsdB, or IsdA and IsdB polypeptide. In certain embodiments an antibody specifically binds both IsdA and IsdB polypeptides. The antibody can be a purified polyclonal antibody, a purified monoclonal antibody, a recombinant polypeptide, or a fragment thereof. In certain aspects the antibody is humanized or human. In still further aspects the antibody is a recombinant antibody segment. In certain aspects a monoclonal antibody includes one or more of 3D8, 4H7, 2A9, 4B9, 7E9, 1B8, 5H8, or 7D4 described in Table 3 below. An antibody can be administered at a dose of 0.1, 0.5, 1, 5, 10, 50, 100 mg or g/kg to 5, 10, 50, 100, 500 mg or μg/kg. The recombinant antibody segment can be operatively coupled to a second recombinant antibody segment. In certain aspects the second recombinant antibody segment binds a second Staphylococcal protein. The method can further comprise administering a second antibody that binds a second Staphylococcal protein. In certain aspects the method further comprises administering an antibiotic.

Embodiments are directed to monoclonal antibody polypeptides, polypeptides having one or more segments thereof, and polynucleotides encoding the same. In certain aspects a polypeptide can comprise all or part of the heavy chain variable region and/or the light chain variable region of IsdA and/or IsdB specific antibodies. In a further aspect, a polypeptide can comprise an amino acid sequence that corresponds to a first, second, and/or third complementary determining regions (CDRs) from the light variable chain and/or heavy variable chain of an antibody, e.g., an IsdA and/or IsdB specific antibody.

In certain embodiments there is provided a recombinant and/or isolated antibody, or antigen-binding portion thereof, that binds to a Staphylococcal IsdA and/or a Staphylococcal IsdB polypeptide wherein the antibody competes for binding of the polypeptide (i.e., IsdA and/or IsdB) with a 3D8, 4H7, 2A9, 4B9, 7E9, 1B8, 5H8, 7D4 and/or 3H11 monoclonal antibody. In some aspects, such an antibody comprises CDR sequences homologous or identical to a CDR from an antibody selected from the group consisting of 3D8, 4H7, 2A9, 4B9, 7E9, 1B8, 5H8, 7D4 and 3H11. For example, the antibody can comprise (a) a first Vh CDR at least 80%, 85%, 90%, 95% or 99% identical to CDR1 of SEQ ID NO: 361, 369, 373, 375, 379, 381, 385, 389, 391 or 395; (b) a second Vh CDR at least 80%, 85%, 90%, 95% or 99% identical to CDR2 of SEQ ID NO: 361, 369, 373, 375, 379, 381, 385, 389, 391 or 395; (c) a third Vh CDR at least 80%, 85%, 90%, 95% or 99% identical to CDR3 of SEQ ID NO: 361, 369, 373, 375, 379, 381, 385, 389, 391 or 395; (d) a first Vl CDR at least 80%, 85%, 90%, 95% or 99% identical to CDR1 of SEQ ID NO: 359, 363, 365, 367, 371, 377, 383, 387, 393 or 397; (e) a second Vl CDR at least 80%, 85%, 90%, 95% or 99% identical to CDR2 of SEQ ID NO: 359, 363, 365, 367, 371, 377, 383, 387, 393 or 397; and (f) a third Vl CDR at least 80%, 85%, 90%, 95% or 99% identical to CDR3 of SEQ ID NO: 359, 363, 365, 367, 371, 377, 383, 387, 393 or 397. In further aspects, the antibody comprises a first, second or third Vh CDR identical to CDR1, CDR2 or CDR3 of SEQ ID NO: 361, 369, 373, 375, 379, 381, 385, 389, 391 or 395 or a sequence differing from the first, second or third Vh CDR of SEQ ID NO: 361, 369, 373, 375, 379, 381, 385, 389, 391 or 395 by one, two or three amino acids. In yet further aspects, the antibody comprises a first, second or third Vl CDR identical to CDR1, CDR2, or CDR3 of SEQ ID NO: 359, 363, 365, 367, 371, 377, 383, 387, 393 or 397 or a sequence differing from the first, second or third Vl CDR of SEQ ID NO: 359, 363, 365, 367, 371, 377, 383, 387, 393 or 397 by one, two or three amino acids. In still further aspects, an antibody of the embodiments comprises a heavy chain variable region less than 99%, 98%, 97%, 95% or 90% identical to a light chain variable region of SEQ ID NO: 361, 369, 373, 375, 379, 381, 385, 389, 391 or 395. In some aspects, an antibody of the embodiments comprises a light chain variable region less than 99%, 98%, 97%, 95% or 90% identical to a light chain variable region of SEQ ID NO: 359, 363, 365, 367, 371, 377, 383, 387, 393 or 397. In some aspects an antibody or fragment thereof of the embodiments comprises a constent or J-region sequences that are substantially non-murine.

In some embodiments, an antibody, or antigen-binding portion thereof is provided, that binds to a Staphylococcal IsdA and/or a Staphylococcal IsdB polypeptide, wherein said antibody, or antigen-binding portion thereof, comprises: (a) a light chain variable region CDR1 sequence comprising the amino acid sequence QZ₁Z₂Z₃Z₄SNGZ₅TY, wherein Z₁ is S or N; Z₂ is L or I; Z₃ is V or L; Z₄ is H or Y and Z₅ is Y, N or K; (b) a light chain variable region CDR2 sequence comprising the amino acid sequence KVS; (c) a light chain variable region CDR3 sequence comprising the amino acid sequence Z₆QZ₇Z₈HZ₉Z₁₀PZ₁₁T, wherein Z₆ is F or S; Z₇ is G, T or S, Z₈ is S or T, Z₉ is V or I, Z₁₀ is absent or, if present, is P and Z₁₁ is Y, L or F; (d) a heavy chain variable region CDR1 sequence comprising the amino acid sequence GZ₁₂TFZ₁₃Z₁₄YZ₁₅, wherein Z₁₂ is Y or F; Z₁₃ is T, G or S, Z₁₄ is E, K, S or D, and Z₁₅ is T, G or S; (e) a heavy chain variable region CDR2 sequence comprising the amino acid sequence IZ₁₆Z₁₇Z₁₈Z₁₉Z₂₀Z₂₁Z₂₂, wherein Z₁₆ is D, N or S; Z₁₇ is P, R or E, Z₁₈ is S, D or N; Z₁₉ is N or G; Z₂₀ is G or S; Z₂₁ is D, S or Y; and Z₂₂ is T or I; and (f) a heavy chain variable region CDR3 sequence comprising the amino acid sequence Z₂₃RZ₂₄Z₂₅Z₂₆Z₂₇Z₂₈Z₂₉Z₃₀Z₃₁Z₃₂, wherein Z₂₃ is A or V; Z₂₄ is absent or, if present, is L or D; Z₂₅ is E or Y; Z₂₆ is G or D; Z₂₇ is V, S or Y; Z₂₈ is L, G or D; Z₂₉ is P, H or A; Z₃₀ is L or F; Z₃₁ is D or A; and Z₃₂ is Y or H.

In some embodiments, an antibody, or antigen-binding portion thereof is provided, that binds to a Staphylococcal IsdA and/or a Staphylococcal IsdB polypeptide, wherein said antibody, or antigen-binding portion thereof, comprises: (a) a light chain variable region CDR1 sequence comprising the amino acid sequence QZ₁Z₂Z₃Z₄SNGZ₅TY, wherein Z₁ is S or N; Z₂ is L or I; Z₃ is V or L; Z₄ is H or Y and Z₅ is Y, N or K; (b) a light chain variable region CDR2 sequence comprising the amino acid sequence KVS; (c) a light chain variable region CDR3 sequence comprising the amino acid sequence Z₆QZ₇Z₈HZ₉Z₁₀PZ₁₁T, wherein Z₆ is F or S; Z₇ is G, T or S, Z₈ is S or T, Z₉ is V or I, Z₁₀ is absent or, if present, is P and Z₁₁ is Y, L or F; (d) a heavy chain variable region CDR1 sequence comprising the amino acid sequence GNAFTNYL, GYSITSDYA or GHSITSGYY; (e) a heavy chain variable region CDR2 sequence comprising the amino acid sequence INPGSGIT, IIFTGAT or ISFDGRN; and (f) a heavy chain variable region CDR3 sequence comprising the amino acid sequence TRELRG, TRLSYSTLDY or SGSANWFAY.

In some embodiments, an antibody, or antigen-binding portion thereof is provided, that binds to a Staphylococcal IsdA polypeptide, wherein said antibody, or antigen-binding portion thereof, comprises: (a) a light chain variable region CDR1 sequence comprising the amino acid sequence QNVGTN; (b) a light chain variable region CDR2 sequence comprising the amino acid sequence SAS; (c) a light chain variable region CDR3 sequence comprising the amino acid sequence QQYNSYPYT; (d) a heavy chain variable region CDR1 sequence comprising the amino acid sequence GYTFTEYT; (e) a heavy chain variable region CDR2 sequence comprising the amino acid sequence IDPSNGDT or IDPDNGDT; and (f) a heavy chain variable region CDR3 sequence comprising the amino acid sequence ARLEGVLPLDY.

In some embodiments, an antibody, or antigen-binding portion thereof is provided, that binds to a Staphylococcal IsdA and/or a Staphylococcal IsdB polypeptide, wherein said antibody, or antigen binding portion thereof, comprises: (a) a light chain variable region CDR1 sequence comprising the amino acid sequence QSLX₁X₂SNGNTY; (b) a light chain variable region CDR2 sequence comprising the amino acid sequence KVS; (c) a light chain variable region CDR3 sequence comprising the amino acid sequence SQX₁THX₂X₃PLT; (d) a heavy chain variable region CDR1 sequence comprising the amino acid sequence GX₁TFTX₂YT; (e) a heavy chain variable region CDR2 sequence comprising the amino acid sequence IDPXNGDT; and (f) a heavy chain variable region CDR3 sequence comprising the amino acid sequence X₁RLEGX₂LPLDY. For example, in certain aspects, the antibody or fragment thereof comprises a sequence wherein: (a) X₁ in the light chain variable CDR1 sequence of (a) is V or L, or a conservative substitution thereof; and/or (b) X₂ in the light chain variable CDR1 sequence of (a) is Y or H, or a conservative substitution thereof; and/or (c) X₁ in the light chain variable CDR3 sequence of (c) is T or S, or a conservative substitution thereof; and/or (d) X₂ in the light chain variable CDR3 sequence of (c) is I or V, or a conservative substitution thereof; and/or (e) X₃ in the light chain variable CDR2 sequence of (c) is absent or is P, or a conservative substitution thereof; and/or (f) X₁ in the heavy chain variable CDR1 sequence of (d) is Y or F, or a conservative substitution thereof; and/or (g) X₂ in the heavy chain variable CDR1 sequence of (d) is E or K, or a conservative substitution thereof; and/or (h) X in the heavy chain variable CDR2 sequence of (e) is S or N, or a conservative substitution thereof; and/or (i) X₁ in the heavy chain variable CDR3 sequence of (f) is A or V, or a conservative substitution thereof; and/or (j) X₂ in the heavy chain variable CDR3 sequence of (f) is V or S, or a conservative substitution thereof. For instance, the antibody, or antigen-binding portion can comprise a sequence wherein: (a) the light chain variable region CDR1 sequence comprises the amino acid sequence QSLVYSNGNTY or QSLLYSNGNTY; (b) the light chain variable region CDR2 sequence comprises the amino acid sequence KVS; (c) the light chain variable region CDR3 sequence comprises the amino acid sequence SQTTHIPLT or SQSTHVPLT; (d) the heavy chain variable region CDR1 sequence comprising the amino acid sequence GYTFTEYT; (e) the heavy chain variable region CDR2 sequence comprising the amino acid sequence IDPSNGDT; and (g) the heavy chain variable region CDR3 sequence comprising the amino acid sequence ARLEGVLPLDY. In some aspects, the antibody, or antigen-binding portion thereof, comprises a sequence wherein the light chain variable region comprises the amino acid sequence of SEQ ID NO: 359, 363 or 371, and the heavy chain variable region comprises the amino acid sequence of SEQ ID NO: 361.

In certain aspects, a polypeptide comprises all or part of an amino acid sequence corresponding to the MAb 3D8 variable light chain amino acid sequence DVVMTQTPLSLPVSLGDQASISCRSSQSLVYSNGNTYLHWFLQKPGQSPKLLIYKVSNR FSGVPDRFSGSGSGTDFTLKISRVEAEDLGVYFCSQTTHIPLTFGAGTKLELK (SEQ ID NO:359). CDRs are indicated in bold underline. CDRs are regions within antibodies where the antibody complements an antigen's shape. Thus, CDRs determine the protein's affinity and specificity for specific antigens. From amino to carboxy terminus the CDRs are CDR1, CDR2, and CDR3. In certain aspects a polypeptide can comprise 1, 2, and/or 3 CDRs from the variable light chain of MAb 3D8.

In a further aspect, a polynucleotide comprises all or part of a nucleic acid sequence corresponding to the MAb 3D8 variable light chain nucleotide sequence GATGTTGTGATGACCCAGACTCCACTCTCCCTGCCTGTCAGTCTTGGAGATCAAGCC TCCATCTCTTGCAGATCTAGTCAGAGCCTTGTATATAGTAATGGAAACACCTATTTA CATTGGTTCCTGCAGAAGCCAGGCCAGTCTCCAAAGCTCCTGATCTACAAAGTTTCC AACCGATTTTCTGGGGTCCCAGACAGGTTCAGTGGCAGTGGATCAGGGACAGATTTC ACACTCAAGATCTCCAGAGTGGAGGCTGAGGATCTGGGAGTTTATTTCTGCTCTCAA ACTACACATATTCCGCTCACGTTCGGTGCTGGGACCAAGCTGGAGCTGAAAC (SEQ ID NO:360).

In certain aspects, a polypeptide comprises all or part of an amino acid sequence corresponding to the MAb 3D8 variable heavy chain amino acid sequence QVQLQQSGAELVRPGTSVKVSCKASGNAFTNYLIEWIKQRPGQGLEWIGVINPGSGITN YNEKFKGKATLTADKSSNTAYMQLSSLSSDDSAVYFCSGSANWFAYWGQGTLVTVSA (SEQ ID NO:385). CDRs are indicated in bold underline. From amino to carboxy terminus the CDRs are CDR1, CDR2, and CDR3. In certain aspects a polypeptide can comprise 1, 2, and/or 3 CDRs from the variable heavy chain of MAb 3D8.

In a further aspect, a polynucleotide comprises all or part of a nucleic acid sequence corresponding to the MAb 3D8 variable heavy chain nucleotide sequence CAGGTCCAGCTGCAGCAGTCTGGAGCTGAACTGGTAAGGCCTGGGACTTCAGTGAA GGTGTCCTGCAAGGCTTICTGGAAACGCCTTCACTAATTATTTAATAGAGTGGATAAA ACAGAGGCCTGGACAGGGCCTTGAGTGGATTGGAGTGATTAATCCTGGAAGTGGAA TTACTAACTACAATGAGAAGTTCAAGGGCAAGGCAACACTGACTGCAGACAAATCC TCCAACACTGCCTACATGCAGCTCAGCAGCCTGTCATCTGATGACTCTGCGGTCTAT TTCTGTTCAGGATCGGCCAACTGGTTTGCTTACTGGGGCCAAGGGACTCTGGTCACT GTCTCTGCA (SEQ ID NO:386).

In certain aspects, a polypeptide comprises all or part of an amino acid sequence corresponding to the MAb 4H7 variable heavy chain amino acid sequence EVQLLQSGPELVKPGTSVKMSCRTSGYTFTEYTMHWVKQSHEKRLEWIGGIDPSNGDT SYNQKFKGKATLTVDKSSSSAYMDLRSLTSVDSAIYYCARLEGVLPLDYWGHGTTLTV SS (SEQ ID NO:361). CDRs are indicated in bold underline. From amino to carboxy terminus the CDRs are CDR1, CDR2, and CDR3. In certain aspects a polypeptide can comprise 1, 2, and/or 3 CDRs from the variable heavy chain of MAb 4H7.

In a further aspect, a polynucleotide comprises all or part of a nucleic acid sequence corresponding to the MAb 4H7 variable heavy chain nucleotide sequence gaggtccagctgctacagtctggacctgaactggtgaagcctgggacttcagtgaagatgtcctgcaggacttctggatacacattcactga atacaccatgcactgggtgaagcagagccatgaaaagagacttgagtggattggaggtattgatcctagcaatggtgatactagctacaacc agaagttcaagggcaaggccacattgactgtagacaagtcctccagctcagcctacatggacctccgcagcctgacatctgtggattctgca atctattactgtgcaagactggaaggagtactaccccttgactactggggccacggcaccactctcacagtctcctcag (SEQ ID NO:362)

In certain aspects, a polypeptide comprises all or part of an amino acid sequence corresponding to the MAb 4H7 variable light chain amino acid sequence DVVMTQTPLSLPVSLGDQASISCRSSQSLVYSNGNTYLHWFLQKPGQSPKLLIYKVSNR FSGVPDRFSGSGSGTDFTLKISRVEAEDLGVYFCSQTTHIPLTFGAGTKLELK (SEQ ID NO:363). CDRs are indicated in bold underline. From amino to carboxy terminus the CDRs are CDR1, CDR2, and CDR3. In certain aspects a polypeptide can comprise 1, 2, and/or 3 CDRs from the variable light chain of MAb 4H7.

In a further aspect, a polynucleotide comprises all or part of a nucleic acid sequence corresponding to the MAb 4H7 Variable light chain nucleotide sequence gatgttgtgatgacccaaactccactctccctgcctgtcagtcttggagatcaagcctccatctcttgcagatctagtcagagccttgtatatagt aatggaaacacctatttacattggttcctgcagaagccaggccagtctccaaagctcctgatctacaaagtttccaaccgattttctggggtcc cagacaggttcagtggcagtggatcagggacagatttcacactcaagatctccagagtggaggctgaggatctgggagtttatttctgctctc aaactacacatattccgctcacgttcggtgctgggaccaagctggagctgaaac (SEQ ID NO:364)

In certain aspects, a polypeptide comprises all or part of an amino acid sequence corresponding to the MAb 2A9 variable light chain amino acid sequence XSXLXXXXXSLPVSLGDQASISXRSSQSLVHSNGNTYLHWFLXKPGQSPKLLIYKVSNR FSGVPGRFSGSGSGTDFTLKISRVEAEDLGVYFCSQSTHVPPLTFGAGTKLELK (SEQ ID NO:365), wherein X is any amino acid. CDRs are indicated in bold underline. From amino to carboxy terminus the CDRs are CDR1, CDR2, and CDR3. In certain aspects a polypeptide can comprise 1, 2, and/or 3 CDRs from the variable light chain of MAb 2A9.

In a further aspect, a polynucleotide comprises all or part of a nucleic acid sequence corresponding to the MAb 2A9 variable light chain nucleotide sequence tgntctgncctcncntnanctcntntatccctgcctgtcagtcttggagatcaagcctccatctctngcagatctagtcagagccttgtacaca gtaatggaaacacctatttacattggttcctgcanaagccaggccagtctccaaagctcctgatctacaaagtttccaaccgattttcnggggt cccaggcaggttcagtggcagtggatcagggacagatttcacactcaagatcagcagagtggaggctgaggatctgggagtttatttctgtt ctcaaagtacacatgttcctccgctcacgttcggtgctgggaccaagctggagctgaagc (SEQ ID NO:366), wherein n is any nucleotide.

In another aspect, a polypeptide comprises all or part of an amino acid sequence corresponding to an alternative MAb 2A9 variable light chain amino acid sequence DVLMTQTPLSLPVSLGDQASISCSSSQNIVHSNGYTYLEWYLQKPGQSP KLLIYKVSNRFSGVPDRFSGSGSGTDFTLKISRVEAEDLGVYFCFQGSHVPYTFGGGTK LEIKR (SEQ ID NO:387). CDRs are indicated in bold underline. From amino to carboxy terminus the CDRs are CDR1, CDR2, and CDR3. In certain aspects a polypeptide can comprise 1, 2, and/or 3 CDRs from the alternative variable light chain of MAb 2A9.

In a further aspect, a polynucleotide comprises all or part of a nucleic acid sequence corresponding to the alternative MAb 2A9 variable light chain nucleotide sequence GATGTTTTGATGACCCAAACTCCACTCTCCCTGCCTGTCAGTCTTGGAGATCAAGCCT CCATCTCTTGCAGCTCTAGTCAGAACATTGTTCATAGTAATGGATACACCTATTTAG AATGGTACCTGCAGAAACCAGGCCAGTCTCCAAAGCTCCTGATCTACAAAGTTTCCA ACCGATTTTCTGGGGTCCCAGACAGGTTCAGTGGCAGTGGTTCAGGGACAGATTTCA CACTCAAGATCAGCAGAGTGGAGGCTGAGGATCTGGGAGTTTATTCTGCTTTCAAG GTTCACATGTTCCGTACACGTTCGGAGGGGGGACCAAGCTGGAAATAAAACG (SEQ ID NO:388).

In still a further aspect, a polypeptide comprises all or part of an amino acid sequence corresponding to the MAb 2A9 variable heavy chain amino acid sequence EVQLVESGGGLVQPGGSLKLSCAASGFTFGSYGMSWVRQTPDKRLELVAIINRNGGST DYPDSVKGRFTISRDNAKNTLYLQMSSLKSEDTAMYNCVREGYGHFDHWGQGTTLTV SS (SEQ ID NO:389). CDRs are indicated in bold underline. From amino to carboxy terminus the CDRs are CDR1, CDR2, and CDR3. In certain aspects a polypeptide can comprise 1, 2, and/or 3 CDRs from the alternative variable heavy chain of MAb 2A9.

In a further aspect, a polynucleotide comprises all or part of a nucleic acid sequence corresponding to the alternative MAb 2A9 variable heavy chain nucleotide sequence GAGGTGCAGCTGGTGGAGTCGGGGGGAGGCTTAGTGCAGCCTGGAGGGTCCCTGAA ACTCTCCTGTGCAGCCTCACTTCGTACTTAGCTATGGCATGTCTTGGGTTCGC CAGACTCCAGACAAGAGGCTGGAGTTGGTCGCAATCATTAATAGAAATGGTGGTAG CACCGATTATCCAGACAGTGTGAAGGGCCGATTCACCATCTCCAGAGACAATGCCA AGAACACCCTGTACCTGCAAATGAGCAGTCTGAAGTCTGAGGACACAGCCATGTAT AACTGTGTAAGAGAGGGTTATGGTCACTTGACCACTGGGGCCAAGGCACCACTCTC ACAGTCTCCTCA (SEQ ID NO:390).

In certain aspects, a polypeptide comprises all or part of an amino acid sequence corresponding to the MAb 4B9 variable light chain amino acid sequence XXXXTQTPLSLPVSLGDQASISCSSSQNIVHSNGYTYLEWYLQKPGQSPKLLIYKVSNRF SGVPDRFSGSGSGTDFTLKISRVEAEDLGVYFCFQGSHVPYTFGGGTKLEIK (SEQ ID NO:367), wherein X is any amino acid. CDRs are indicated in bold underline. From amino to carboxy terminus the CDRs are CDR1, CDR2, and CDR3. In certain aspects a polypeptide can comprise 1, 2, and/or 3 CDRs from the variable light chain of MAb 4B9.

In a further aspect, a polynucleotide comprises all or part of a nucleic acid sequence corresponding to the MAb 4B9 variable light chain nucleotide sequence tgnngnttnntgacccaaactccactctccctgcctgtcagtcttggagatcaagcctccatctcttgcagctctagtcagaacattgttcatag taatggatacacctatttagaatggtacctgcagaaaccaggccagtctccaaagctcctgatctacaaagtttccaaccgattttctggggtc ccagacaggttcagtggcagtggttcagggacagatttcacactcaagatcagcagagtggaggctgaggatctgggagtttatttctgcttt caaggttcacatgttccgtacacgttcggaggggggaccaagctggaaataaaac (SEQ ID NO:368), wherein n is any nucleotide.

In certain aspects, a polypeptide comprises all or part of an amino acid sequence corresponding to the MAb 4B9 variable heavy chain amino acid sequence DVQLQESGPGLVKPSQSLSLTCTVTGYSITSDYAWNWIRQFPGNKLEWLGSIIFTGATD YNPSLKSXISITRDTSKNQFFLHLTXMTTEDTATYYCTRELRGWGQGTTLTVSS (SEQ ID NO:369). CDRs are indicated in bold underline. From amino to carboxy terminus the CDRs are CDR1, CDR2, and CDR3. In certain aspects a polypeptide can comprise 1, 2, and/or 3 CDRs from the variable heavy chain of MAb 4B9.

In a further aspect, a polynucleotide comprises all or part of a nucleic acid sequence corresponding to the MAb 4B9 variable heavy chain nucleotide sequence gatgtgcagcttcaggagtcgggacctggcctggtgaagccttctcagtctctgtccctcacctgcactgtcactggctactcaatcaccagt gattatgcctggaactggatccggcagtttccaggaaacaaactggagtggttgggctccataatcttcactggtgccactgactacaaccca tctctcaaaagtngaatctctatcactcgagacacatccaagaaccagttcttcctccttgacttntatgactactgaggacacacat attattgtacaagagaacttagaggctggggccaaggcaccactctcacagtctcctcag (SEQ ID NO:370)

In certain aspects, a polypeptide comprises all or part of an amino acid sequence corresponding to the MAb 7E9 variable light chain amino acid sequence DVVMTQTPLSLPVSLGDQASISCRSSQSLLYSNGNTYLHWYLQKPGQSPKLLIYKVSNR FSGVPDRFSASGSGTDFTLKISRVEAEDLGVYFCSQSTHVPLTFGAGTKLELK (SEQ ID NO:371). CDRs are indicated in bold underline. From amino to carboxy terminus the CDRs are CDR1, CDR2, and CDR3. In certain aspects a polypeptide can comprise 1, 2, and/or 3 CDRs from the variable light chain of MAb 7E9.

In a further aspect, a polynucleotide comprises all or part of a nucleic acid sequence corresponding to the MAb 7E9 variable light chain nucleotide sequence gatgttgtgatgacccaaactccactctccctgcctgtcagtcttggagatcaagcctccatctcttgcagatctagtcagagcctattatacagt aatggaaacacctatttacattggtacctgcagaagccaggccagtctccaaagctcctgatctacaaagtttccaaccgattttctggggtcc cagacaggttcagtgccagtggatcagggacagatttcacactcaagatcagcagagtggaggctgaggatctgggagtttatttctgttctc aaagtacacatgttccgctcacgttcggtgctgggaccaagctggagctgaaac (SEQ ID NO:372).

In certain aspects, a polypeptide comprises all or part of an amino acid sequence corresponding to the MAb 7E9 variable heavy chain amino acid sequence SDVQLQESGPGLVKPSQSLSLTCSVTGHSITSGYYWNWIRQFPGNKLEWMGYISFDGR NKYNPSLKNRISITRDTSKNQFFLKLNSVTSEDTATYFCTRLSYSTLDYWGQGTSVTVSS (SEQ ID NO:391). CDRs are indicated in bold underline. From amino to carboxy terminus the CDRs are CDR1, CDR2, and CDR3. In certain aspects a polypeptide can comprise 1, 2, and/or 3 CDRs from the variable heavy chain of MAb 7E9.

In a further aspect, a polynucleotide comprises all or part of a nucleic acid sequence corresponding to the MAb 7E9 variable heavy chain nucleotide sequence TCTGATGTACAGCTTCAGGAGTCAGGACCTGGCCTCGTGAAACCTTCTCAGTCTCTG TCTCTCACCTGCTCTGTCACTGGCCACTCCATCACCAGTGGTTATTACTGGAACTGGA TCCGGCAGTTTCCAGGAAACAAACTGGAATGGATGGGCTACATAAGTTTCGACGGT CGCAATAAGTACAACCCATCTCTCAAAAATCGAATCTCCATCACTCGTGACACATCT AAGAACCAGTTITTTCCTGAAGTTGAATTCTGTGACCTCTGAGGACACAGCTACATAT TTCTGTACAAGACTAAGTTACTCTACTCTGGACTACTGGGGTCAAGGAACCTCAGTC ACCGTCTCCTCA (SEQ ID NO:392).

In certain aspects, a polypeptide comprises all or part of an amino acid sequence corresponding to the MAb 1B8 variable light chain amino acid sequence DVVMTQTPLSLPVSLGDQASISCRSSQSLVHSNGNTYLHWYLQKPGQSPKLLIYKVSSR FSGVPDRFSGSGSGTDFTLKISRVEAEDLGVYFCSQSTHVPYTFGGGTKLEIKR (SEQ ID NO:393). CDRs are indicated in bold underline. From amino to carboxy terminus the CDRs are CDR1, CDR2, and CDR3. In certain aspects a polypeptide can comprise 1, 2, and/or 3 CDRs from the variable light chain of MAb 1B8.

In a further aspect, a polynucleotide comprises all or part of a nucleic acid sequence corresponding to the MAb 1B8 variable light chain nucleotide sequence GATGTTGTGATGACCCAAACTCCACTCTCCCTGCCTGTCAGTCTTGGAGATCAAGCC TCCATCTCTTGCAGATCTAGTCAGAGCCTTGTACACAGTAATGGAAACACCTATTTA CATTGGTACCTGCAGAAGCCAGGCCAGTCTCCAAAGCTCCTGATCTACAAAGTTTCC AGCCGATTTTCTGGGGTCCCAGACAGGTTCAGTGGCAGTGGATCAGGGACAGATTTC ACACTCAAGATCAGCAGAGTGGAGGCTGAGGATCTGGGAGTTTATTTCTGCTCTCAA AGTACACATGTTCCGTACACGTTCGGAGGGGGGACCAAGCTGGAAATAAAACG (SEQ ID NO:394)

In certain aspects, a polypeptide comprises all or part of an amino acid sequence corresponding to the MAb 1B8 variable heavy chain amino acid sequence EVQLQESGPELVKPGTSVWISCKTSGFTFTKYTMHWVKQSHGKTLEWIGGIDPNNGDT SYNQKFKDKATLTVDKSSSTAYMELRSLTSEDSAVFFCVRLEGSLPLDYWGQGTTLTV SS (SEQ ID NO:373). CDRs are indicated in bold underline. From amino to carboxy terminus the CDRs are CDR1, CDR2, and CDR3. In certain aspects a polypeptide can comprise 1, 2, and/or 3 CDRs from the variable heavy chain of MAb 1B8.

In a further aspect, a polynucleotide comprises all or part of a nucleic acid sequence corresponding to the MAb 1B8 variable heavy chain nucleotide sequence gaggtccagctgcaagagtctggacctgaactggtgaagcctgggacttcagtgtggatatcctgcaagacttctggattcacattcactaaa tacaccatgcactgggtgaagcagagccatggaaagacccttgagtggattggaggtattgatcctaacaatggtgatactagttacaacca gaagttcaaggacaaggccacattgactgtagacaagtcctccagcacagcctacatggaactccgcagcctgacatctgaagattctgca gtctttttctgtgtaagactggaagggtcactgccccttgactactggggccaaggcaccactctcacagtctcctcag (SEQ ID NO:374).

In another aspect, a polypeptide comprises all or part of an amino acid sequence corresponding to an alternative MAb 1B8 variable heavy chain amino acid sequence EVQLVESGGGLVKPGGSLKISCAASGFTFSDYSMYWVRQTPEKRLEWVATISEGGSYI NYPDNVKGRFTISRDNAKNNLYLQMSSLKSEDAAMYYCARDYDYDAFAYWGQGTLV TVS (SEQ ID NO:395). CDRs are indicated in bold underline. From amino to carboxy terminus the CDRs are CDR1, CDR2, and CDR3. In certain aspects a polypeptide can comprise 1, 2, and/or 3 CDRs from the alternative variable heavy chain of MAb 1B8.

In a further aspect, a polynucleotide comprises all or part of a nucleic acid sequence corresponding to the alternative MAb 1B8 variable heavy chain nucleotide sequence GAAGTGCAGCTGGTGGAGTCTGGGGGAGGCTTAGTGAAGCCTGGAGGGTCCCTGAA AATCTCCTGTGCAGCCTCTGGATTCACTTTCAGTGACTATTCCATGTATTGGGTTCGC CAGACTCCGGAAAAGAGGCTGGAGTGGGTCGCAACCATTAGTGAAGGTGGTAGTTA CATCAACTATCCAGACAATGTGAAGGGGCGATTCACCATCTCCAGAGACAATGCCA AGAACAACCTGTACCTGCAAATGAGCAGTCTGAAGTCTGAGGACGCAGCCATGTAT TACTGTGCAAGAGACTATGATTACGACGCTTTTGCTTACTGGGGCCAAGGGACTCTG GTCACTGTCTCTG (SEQ ID NO:396).

In certain aspects, a polypeptide comprises all or part of an amino acid sequence corresponding to the MAb 5H8 variable heavy chain amino acid sequence GLTGEPGTSVKMSCRTSGYTFTEYTMHWVKQSHEKRLEWIGGIDPSNGDTSYNQKFK GKATLTVDKSSSSAYMDLRSLTSVDSAIYYCARLEGVLPLDYWGHGTTLTVSS (SEQ ID NO:375). CDRs are indicated in bold underline. From amino to carboxy terminus the CDRs are CDR1, CDR2, and CDR3. In certain aspects a polypeptide can comprise 1, 2, and/or 3 CDRs from the variable heavy chain of MAb 5H8.

In a further aspect, a polynucleotide comprises all or part of a nucleic acid sequence corresponding to the MAb 5H8 variable heavy chain nucleotide sequence ggactgactggtgagcctgggacttcagtgaagatgtcctgcaggacttctggatacacattcactgaatacaccatgcactgggtgaagca gagccatgaaaagagacttgagtggattggaggtattgatcctagcaatggtgatactagctacaaccagaagttcaagggcaaggccaca ttgactgtagacaagtcctccagctcagcctacatggacctccgcagcctgacatctgtggattctgcaatctattactgtgcaagactggaag gagtactaccccttgactactggggccacggcaccactctcacagtctcctcag (SEQ ID NO:376)

In certain aspects, a polypeptide comprises all or part of an amino acid sequence corresponding to the MAb 5H8 variable light chain amino acid sequence DIVMTQSQKFMSTSVRDRVAVTCKASQNVGTNVAWYQQKPGQSPKALIYSASYRYSG VPDRFTGSGSGTDFTLTISNVQSEDLAEYFCQQYNSYPYTFGGGTKLEVK (SEQ ID NO:377). CDRs are indicated in bold underline. From amino to carboxy terminus the CDRs are CDR1, CDR2, and CDR3. In certain aspects a polypeptide can comprise 1, 2, and/or 3 CDRs from the variable light chain of MAb 5H8.

In a further aspect, a polynucleotide comprises all or part of a nucleic acid sequence corresponding to the MAb 5H8 variable light chain nucleotide sequence gacattgtgatgacccagtctcaaaaattcatgtccacatcagtaagagacagggtcgccgtcacctgcaaggccagtcagaatgtgggtac taatgtagcctggtatcaacagaaaccaggtcaatctcctaaagcactgatttactcggcatcctaccggtacagtggagtccctgatcgcttc acaggcagtggatctgggacagatttcactctcaccatcagcaatgtgcagtctgaagacttggcagagtatttctgtcagcagtataacagc tatccgtacacgttcggaggggggaccaagctggaagtaaaac (SEQ ID NO:378).

In certain aspects, a polypeptide comprises all or part of an amino acid sequence corresponding to the MAb 7D4 variable light chain amino acid sequence DVGMTQTPLSLPVSLGDQASISCGSSQSLLHSNGKTYLHWYLQKPGQSPKLLIYKVSNR FSGVPDRFSGSGSGTYFTLKITRVEAEDLGVYFCSQTTHVPFTFGSGTKLEIK (SEQ ID NO:397). CDRs are indicated in bold underline. From amino to carboxy terminus the CDRs are CDR1, CDR2, and CDR3. In certain aspects a polypeptide can comprise 1, 2, and/or 3 CDRs from the variable light chain of MAb 7D4.

In a further aspect, a polynucleotide comprises all or part of a nucleic acid sequence corresponding to the MAb 7D4 variable light chain nucleotide sequence GATGTTGGGATGACCCAAACTCCTCTCTCCCTGCCTGTCAGTCTTGGAGATCAAGCC TCCATCTCTITGCGGATCTAGTCAGAGCCTTCTACACAGTAATGGAAAGACCTATTTA CACTGGTACCTGCAGAAGCCAGGCCAGTCTCCAAAGCTCCTGATCTACAAAGTTTCC AACCGATTTTCTGGGGTCCCCGACAGGTTCAGTGGCAGTGGATCAGGGACATATTTC ACACTCAAGATCACCAGAGTGGAGGCTGAGGATCTGGGAGTTTATTICTGCTCTCAA ACTACCCATGTTCCATTCACGTTCGGCTCGGGGACAAAGTTGGAAATAAAAC (SEQ ID NO:398).

In certain aspects, a polypeptide comprises all or part of an amino acid sequence corresponding to the MAb 7D4 variable heavy chain amino acid sequence EVQLQESGPELVKPGTSVWISCKTSGFTFTKYTMHWVKQSHGKTLEWIGGIDPNNGDT SYNQKFKDKATLTVDKSSSTAYMELRSLTSEDSAVFFCVRLEGSLPLDYWGQGTTLTV SS (SEQ ID NO:379). CDRs are indicated in bold underline. From amino to carboxy terminus the CDRs are CDR1, CDR2, and CDR3. In certain aspects a polypeptide can comprise 1, 2, and/or 3 CDRs from the variable heavy chain of MAb 7D4.

In a further aspect, a polynucleotide comprises all or part of a nucleic acid sequence corresponding to the MAb 7D4 variable heavy chain nucleotide sequence gaggtccagctgcaagagtctggacctgaactggtgaagcctgggacttcagtgtggatatcctgcaagacttctggattcacattcactaaa tacaccatgcactgggtgaagcagagccatggaaagacccttgagtggattggaggtattgatcctaacaatggtgatactagttacaacca gaagttcaaggacaaggccacattgactgtagacaagtcctccagcacagcctacatggaactccgcagcctgacatctgaagattctgca gtctttttctgtgtaagactggaagggtcactgccccttgactactggggccaaggcaccactctcacagtctcctcag (SEQ ID NO:380).

In certain aspects, a polypeptide comprises all or part of an amino acid sequence corresponding to the MAb 3H11 variable heavy chain amino acid sequence SLDLTGEPGASVKMSCRTSGYTFTEYTMHWVKQSHEKSLEWIGGIDPDNGDTSFNQKF KGKATLTVDKSSSTAYMELRSLTYDDTAIYLCARLEGVLPLDYWGQGTTLTVSS (SEQ ID NO:381). CDRs are indicated in bold underline. From amino to carboxy terminus the CDRs are CDR1, CDR2, and CDR3. In certain aspects a polypeptide can comprise 1, 2, and/or 3 CDRs from the variable heavy chain of MAb 3H11.

In a further aspect, a polynucleotide comprises all or part of a nucleic acid sequence corresponding to the MAb 3H11 variable heavy chain nucleotide sequence agtctggacctgactggtgagcctggggcttcagtgaagatgtcctgcaggacttctggatacacattcactgaatacaccatgcactgggtg aagcagagccatgaaaagagccttgaatggattggaggtattgatcctgacaatggtgatactagcttcaaccagaagttcaagggcaagg ccacattgactgtagacaagtcctccagcacagcctacatggagctccgcagcctgacatatgacgatactgcaatctatctctgtgcaagac tggaaggagtactcccccttgactactggggccaaggcaccactctcacagtctcctcag (SEQ ID NO:382)

In certain aspects, a polypeptide comprises all or part of an amino acid sequence corresponding to the MAb 3H11 variable light chain amino acid sequence DIVMTQSQKFMSTSVRDRVAVTCKASQNVGTNVAWYQQKPGQSPKALIYSASYRYSG VPDRFTGSGSGTDFTLTISNVQSEDLAEYFCQQYNSYPYTFGGGTKLEVK (SEQ ID NO:383), CDRs are indicated in bold underline. From amino to carboxy terminus the CDRs are CDR1, CDR2, and CDR3. In certain aspects a polypeptide can comprise 1, 2, and/or 3 CDRs from the variable light chain of MAb 3H11.

In a further aspect, a polynucleotide comprises all or part of a nucleic acid sequence corresponding to the MAb 3H11 variable light chain nucleotide sequence gacattgtgatgacccagtctcaaaaattcatgtccacatcagtaagagacagggtcgccgtcacctgcaaggccagtcagaatgtgggtac taatgtagcctggtatcaacagaaaccaggtcaatctcctaaagcactgatttactcggcatcctaccggtacagtggagtcctgatgcttc acaggcagtggatctgggacagatttcactctcaccatcagcaatgtgcagtctgaagacttggcagagtatttctgtcagcagtataacag tatccgtacacgttcggaggggggaccaagctggaagtaaaac (SEQ ID NO:384)

In a further aspect, 1, 2, and/or 3 CDRs from the light and/or heavy chain variable region of a MAb can be comprised in a humanized antibody or variant thereof.

In certain aspects, a monoclonal antibody does not bind IsdB. In a further aspect, a monoclonal antibody specifically binds IsdA with minimal cross-reactivity to IsdB on western blot analysis. In certain aspects the monoclonal antibody specifically binds the heme binding region of IsdA, IsdB, or IsdA and IsdB. In still a further aspect, a monoclonal antibody is non-opsonophagocytic. In certain aspects, a monoclonal antibody does not specifically compete with a 2H2.BE11 monoclonal antibody produced by the hybridoma having ATCC accession number PTA-7124. In still a further aspect, a monoclonal specifically binds IsdA and IsdB, but does not specifically compete for binding of a 2H2.BE11 monoclonal antibody produced by the hybridoma having ATCC accession number PTA-7124.

Certain aspects are directed to methods of treating a subject having or suspected of having a Staphylococcus infection comprising administering to a patient having or suspected of having a Staphylococcus infection an effective amount of a purified antibody that specifically binds a Staphylococcal IsdA, IsdB, or IsdA and IsdB polypeptide.

In a further aspect methods are directed to treating a subject at risk of a Staphylococcus infection comprising administering to a patient at risk of a Staphylococcus infection an effective amount of an antibody that binds a Staphylococcal IsdA, IsdB, or IsdA and IsdB polypeptide prior to infection with Staphylococcus.

Certain embodiments are directed to a purified antibody or binding polypeptide composition comprising an antibody or polypeptide that specifically binds a peptide segment as described above. In certain aspects the antibody or polypeptide binds a 5 to 50 amino acid peptide having a sequence that is at least 90% identical to a 5 to 50 amino acid sequence of staphylococcal IsdA or IsdB polypeptide (SEQ ID NO:1-3, 128, 129, 252, or 253). In still further aspects the antibody or polypeptide binds a peptide having 90, 95, 99, or 100% identity to one or more of SEQ ID NO:4-127, 130-251, or 254-354. In certain aspects antibodies of the invention can be encoded by a nucleic acid. Antibodies of the invention can be monoclonal antibodies or a recombinant segment of an antibody. In certain aspects the antibody binds both IsdA and IsdB or portions thereof.

Certain embodiments are directed to a antibody or binding polypeptide composition comprising an isolated and/or recombinant antibody or polypeptide that specifically binds a peptide segment as described above. In certain aspects the antibody or polypeptide has a sequence that is, is at least, or is at most 80, 85, 90, 95, 96, 97, 98, 99, or 100% identical (or any range derivable therein) to all or part of any monoclonal antibody provided herein (SEQ ID NOs 359, 361, 363, 365, 367, 369, 371, 373, 375, 377, 379, 381, or 383). In still further aspects the isolated and/or recombinant antibody or polypeptide has, has at least, or has at most 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more contiguous amino acids from any of SEQ ID NOs: 359. 361, 363, 365, 367, 369, 371, 373, 375, 377, 379, 381, or 383 or a combination of such SEQ ID NOs. In certain aspects the antibody binds both IsdA and IsdB or portions thereof.

In certain aspects antibodies of the invention can be encoded by a nucleic acid. Antibodies of the invention can be monoclonal antibodies or a recombinant segment of an antibody. Moreover, in some embodiments, a polypeptide or antibody containing one or more of all or part of SEQ ID NOs 359. 361, 363, 365, 367, 369, 371, 373, 375, 377, 379, 381, or 383 is chimeric or humanized.

Certain aspects are directed to a purified monoclonal antibody that specifically binds a peptide as described above. In particular, a monoclonal antibody will bind a 5 to 50 amino acid peptide having a sequence that is at least 90% identical to a 5 to 50 amino acid sequence of staphylococcal IsdB polypeptide (SEQ ID NO:2, 128-354).

Other aspects are directed to a recombinant antibody segment that specifically binds a peptide segment as described above. In particular, the peptide segment is a 5 to 50 amino acid peptide having a sequence that is at least 90% identical to a 5 to 50 amino acid sequence of staphylococcal IsdB polypeptide (SEQ ID NO:2, 128-354).

Still other aspects are directed to a purified antibody composition comprising antibodies that specifically bind an amino acid sequence that is at least 60, 70, 80, 90 or 100% identical to SEQ ID NO:17-117, 143-251, 264-354.

In additional embodiments, there are pharmaceutical compositions comprising one or more polypeptides or antibodies or antibody fragments that are discussed herein. Such a composition may or may not contain additional active ingredients.

In certain embodiments there is a pharmaceutical composition consisting essentially of a polypeptide comprising one or more antibody fragments discussed herein. It is contemplated that the composition may contain non-active ingredients.

Certain aspects are directed to methods of treating a patient having or suspected of having a Staphylococcus infection comprising administering an effective amount of an isolated antibody that specifically binds a 5 to 50 amino acid peptide having a sequence that is at least 90% identical to a 5 to 50 amino acid sequence of staphylococcal IsdB polypeptide (SEQ ID NO:2, 128-354). The method can further comprise administering an effective amount of a second anti-bacterial agent. The second anti-bacterial agent can be selected from an antibiotic, a vaccine, or a second anti-staphylococcus antibody.

Other aspects are directed to methods of protecting a subject from Staphylococcus infection comprising administering an isolated antibody that specifically binds a 5 to 50 amino acid peptide having a sequence that is at least 80, 85, 90, or 100% identical to a 5 to 50 amino acid sequence of staphylococcal IsdB polypeptide (SEQ ID NO:2, 128-354).

Still other aspects are directed to an anti-bacterial composition comprising one or more isolated antibody that specifically binds a 5 to 50 amino acid peptide having a sequence that is at least 90% identical to a 5 to 50 amino acid sequence of staphylococcal IsdA or IsdB polypeptide (SEQ ID NO:1-354).

Certain aspects are directed to nucleic acid molecules encoding a heavy chain variable regions and/or light chain variable regions of an antibody that specifically binds IsdA, IsdB, IsdA and IsdB, or a peptide as described above.

Other aspects are directed to pharmaceutical compositions comprising an effective anti-bacterial amount of an antibody that specifically binds to a peptide described above and a pharmaceutically acceptable carrier.

The term “providing” is used according to its ordinary meaning to indicate “to supply or furnish for use.” In some embodiments, the protein is provided directly by administering a composition comprising antibodies or fragments thereof of the invention.

The subject typically will have (e.g., diagnosed with a persistent staphylococcal infection), will be suspected of having, or will be at risk of developing a staphylococcal infection. Compositions of the present invention include IsdA and/or IsdB binding polypeptides in amounts effective to achieve the intended purpose—treatment or protection of Staphylococcal infection. The term “binding polypeptide” refers to a polypeptide that specifically binds to a target molecule, such as the binding of an antibody to an antigen. Binding polypeptides may but need not be derived from immunoglobulin genes or fragments of immunoglobulin genes. More specifically, an effective amount means an amount of active ingredients necessary to provide resistance to, amelioration of, or mitigation of infection. In more specific aspects, an effective amount prevents, alleviates or ameliorates symptoms of disease or infection, or prolongs the survival of the subject being treated. Determination of the effective amount is well within the capability of those skilled in the art, especially in light of the detailed disclosure provided herein. For any preparation used in the methods of the invention, an effective amount or dose can be estimated initially from in vitro, cell culture, and/or animal model assays. For example, a dose can be formulated in animal models to achieve a desired response. Such information can be used to more accurately determine useful doses in humans.

Compositions can comprise an antibody or a cell that binds IsdA and/or IsdB. An antibody can be an antibody fragment, a humanized antibody, a monoclonal antibody, a single chain antibody or the like. In certain aspects, the IsdA and/or IsdB antibody is elicited by providing an IsdA and/or IsdB peptide or antigen or epitope that results in the production of an antibody that binds IsdA and/or IsdB in the subject. The IsdA and/or IsdB antibody is typically formulated in a pharmaceutically acceptable composition. The IsdA and/or IsdB antibody composition can further comprise at least 1, 2, 3, 4, 5, 6, 7, 8, 9, 10, 11, 12, 13, 14, 15, 16, 17, 18, or 19 for more staphylococcal antigens or immunogenic fragments thereof. Staphylococcal antigens include, but are not limited to all or a segment of Eap, Ebh, Emp, EsaB, EsaC, EsxA, EsxB, SdrC, SdrD, SdrE, ClfA, ClfB, Coa, Hla (e.g., H35 mutants), IsdC, SasF, vWa, SpA and variants thereof (See U.S. Provisional Application Ser. Nos. 61/166,432, filed Apr. 3, 2009; 61/170,779, filed Apr. 20, 2009; and 61/103,196, filed Oct. 6, 2009; each of which is incorporated herein by reference in their entirety), vWh, 52 kDa vitronectin binding protein (WO 01/60852), Aaa (GenBank CAC80837), Aap (GenBank accession AJ249487), Ant (GenBank accession NP_{—}372518), autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/PisA, laminin receptor, Lipase GehD, MAP, Mg2+ transporter, MHC II analogue (U.S. Pat. No. 5,648,240), MRPII, Npase, RNA III activating protein (RAP), SasA, SasB, SasC, SasD, SasK, SBI, SdrF (WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein (see PCT publications WO2007/113222, WO2007/113223, WO2006/032472, WO2006/032475, WO2006/032500, each of which is incorporated herein by reference in their entirety). The staphylococcal antigen, or immunogenic fragment or segment can be administered concurrently with the IsdA and/or IsdB antibody. The staphylococcal antigen or immunogenic fragment and the IsdA and/or IsdB antibody can be administered in the same or different composition and at the same or different times.

The IsdA and/or IsdB antibody composition can further comprise antibodies, antibody fragments or antibody subfragments to at least 1, 2, 3, 4, 5, 6, 7, 8, 9, 10, 11, 12, 13, 14, 15, 16, 17, 18, or 19 of more staphylococcal antigens or immunogenic fragments thereof. Staphylococcal antigens to which such antibodies, antibody fragments of antibody subfragments are directed include, but are not limited to all or a segment of Eap, Ebh, Emp, EsaB, EsaC, EsxA, EsxB, SdrC, SdrD, SdrE, ClfA, ClfB, Coa, Hla (e.g., H35 mutants), IsdC, SasF, vWa, SpA and variants thereof (See U.S. Provisional Application Ser. Nos. 61/166,432, filed Apr. 3, 2009; 61/170,779, filed Apr. 20, 2009; and 61/103,196, filed Oct. 6, 2009; each of which is incorporated herein by reference in their entirety), vWh, 52 kDa vitronectin binding protein (WO 01/60852), Aaa (GenBank CAC80837), Aap (GenBank accession AJ249487), Ant (GenBank accession NP_{—}372518), autolysin glucosaminidase, autolysin amidase, Cna, collagen binding protein (U.S. Pat. No. 6,288,214), EFB (FIB), Elastin binding protein (EbpS), EPB, FbpA, fibrinogen binding protein (U.S. Pat. No. 6,008,341), Fibronectin binding protein (U.S. Pat. No. 5,840,846), FnbA, FnbB, GehD (US 2002/0169288), HarA, HBP, Immunodominant ABC transporter, IsaA/PisA, laminin receptor, Lipase GehD, MAP, Mg²⁺ transporter, MHC II analogue (U.S. Pat. No. 5,648,240), MRPII, Npase, RNA III activating protein (RAP), SasA, SasB, SasC, SasD, SasK, SBI, SdrF (WO 00/12689), SdrG/Fig (WO 00/12689), SdrH (WO 00/12689), SEA exotoxins (WO 00/02523), SEB exotoxins (WO 00/02523), SitC and Ni ABC transporter, SitC/MntC/saliva binding protein (U.S. Pat. No. 5,801,234), SsaA, SSP-1, SSP-2, and/or Vitronectin binding protein (see PCT publications WO2007/113222, WO2007/113223, WO2006/032472, WO2006/032475, WO2006/032500, each of which is incorporated herein by reference in their entirety). The antibodies, antibody fragments or antibody subfragments to other staphylococcal antigens or immunogenic fragments thereof can be administered concurrently with the IsdA and/or IsdB antibody. The antibodies, antibody fragments or antibody subfragments to other staphylococcal antigens or immunogenic fragments thereof can be administered in the same or different composition to the IsdA and/or IsdB antibody and at the same or different times.

As used herein, the term “modulate” or “modulation” encompasses the meanings of the words “inhibit.” “Modulation” of activity is a decrease in activity. As used herein, the term “modulator” refers to compounds that effect the function of a Staphylococcal bacteria, including potentiation, inhibition, down-regulation, or suppression of a protein, nucleic acid, gene, organism or the like.

Embodiments of the invention include compositions that contain or do not contain a bacterium. A composition may or may not include an attenuated or viable or intact staphylococcal bacterium. In certain aspects, the composition comprises a bacterium that is not a Staphylococci bacterium or does not contain Staphylococci bacteria. In certain embodiments a bacterial composition comprises an isolated or recombinantly expressed IsdA and/or IsdB antibody or a nucleic acid encoding the same. In still further aspects, the IsdA and/or IsdB antibody is multimerized, e.g., a dimer, a trimer, a tertramer, etc.

In certain aspects of the invention, a peptide or an antigen or an epitope of the invention can be presented as multimers of 1, 2, 3, 4, 5, 6, 7, 8, 9, 10 or more peptide segments or peptide mimetics.

The term “isolated” can refer to a nucleic acid or polypeptide that is substantially free of cellular material, bacterial material, viral material, or culture medium (when produced by recombinant DNA techniques) of their source of origin, or chemical precursors or other chemicals (when chemically synthesized). Moreover, an isolated compound refers to one that can be administered to a subject as an isolated compound; in other words, the compound may not simply be considered “isolated” if it is adhered to a column or embedded in an agarose gel. Moreover, an “isolated nucleic acid fragment” or “isolated peptide” is a nucleic acid or protein fragment that is not naturally occurring as a fragment and/or is not typically in the functional state.

Compositions of the invention, such as antibodies, peptides, antigens or immunogens, may be conjugated or linked covalently or noncovalently to other moieties such as adjuvants, proteins, peptides, supports, fluorescence moieties, or labels. The term “conjugate” or “immunoconjugate” is broadly used to define the operative association of one moiety with another agent and is not intended to refer solely to any type of operative association, and is particularly not limited to chemical “conjugation.” Recombinant fusion proteins are particularly contemplated.

The term “IsdA and/or IsdB antibody” refers to polypeptides that bind IsdA, IsdB, or IsdA and IsdB proteins from staphylococcus bacteria.

In certain aspects, an IsdA and/or IsdB peptide or antigen or epitope can have a sequence that is at least 85%, preferably at least 90%, more preferably at least 95%, and most preferably at least 98% or 99% or more identical to an amino acid sequence of segment of IsdA or IsdB or of a consensus sequence designed by the inventors. The term “identity” refers to a polypeptide that has a sequence that has a certain percentage of amino acids that are identical to a reference polypeptide. Typically the amino acid sequences are aligned and identity can then be calculated by (1−[(total residues aligned−number of identical residues) divided by the total number of residues]) multiplied by one hundred.

The polypeptides described herein may include the following, or at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, or more contiguous amino acids, or any range derivable therein, of one or more of SEQ ID NO:1-354. The compositions may be formulated in a pharmaceutically acceptable composition.

In further aspects of the invention a composition may be administered more than one time to the subject, and may be administered 1, 2, 3, 4, 5, 6, 7, 8, 9, 10, 15, 20 or more times. The administration of the compositions include, but is not limited to oral, parenteral, subcutaneous and intravenous administration, or various combinations thereof, including inhalation or aspiration.

Compositions of the invention are typically administered to human subjects, but administration to other animals that are capable of providing a therapeutic benefit against a staphylococcus bacterium are contemplated, particularly cattle, horses, goats, sheep and other domestic animals, i.e., mammals. In further aspects the staphylococcus bacterium is a Staphylococcus aureus. In still further aspects, the methods and compositions of the invention can be used to prevent, ameliorate, reduce, or treat infection of tissues or glands, e.g., mammary glands, particularly mastitis and other infections. Other methods include, but are not limited to prophylatically reducing bacterial burden in a subject not exhibiting signs of infection, particularly those subjects suspected of or at risk of being colonized by a target bacteria, e.g., patients that are or will be at risk or susceptible to infection during a hospital stay, treatment, and/or recovery.

Still further embodiments include methods for providing a subject a protective or therapeutic composition against a staphylococcus bacterium comprising administering to the subject an effective amount of a composition including (i) an IsdA and/or IsdB antibody; or, (ii) a nucleic acid molecule encoding the same, or (iii) administering an IsdA and/or IsdB antibody with any combination or permutation of bacterial proteins described herein.

The embodiments in the Example section are understood to be embodiments of the invention that are applicable to all aspects of the invention, including compositions and methods.

The use of the term “or” in the claims is used to mean “and/or” unless explicitly indicated to refer to alternatives only or the alternatives are mutually exclusive, although the disclosure supports a definition that refers to only alternatives and “and/or.” It is also contemplated that anything listed using the term “or” may also be specifically excluded.

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

So that the matter in which the above-recited features, advantages and objects of the invention as well as others which will become clear are attained and can be understood in detail, more particular descriptions and certain embodiments of the invention briefly summarized above are illustrated in the appended drawings. These drawings form a part of the specification. It is to be noted, however, that the appended drawings illustrate certain embodiments of the invention and therefore are not to be considered limiting in their scope.

FIG. 1. Contribution of iron-regulated surface determinants (Isd) to Staphylococcus aureus abscess formation and lethal infection in mice. (A-F) Six week old BALB/c mice were infected by retroorbital injection with 1×10⁷ CFU S. aureus Newman. Four days following challenge, mice were killed and kidneys removed for histopathology (A-E) or bacterial load measurements (F). For histopathology, kidneys were fixed in formaldehyde, thin sectioned, stained with hematoxylene-eosin and viewed by light microscopy. For tissue homogenization, kidneys were mechanically disrupted into PBS, 1% Triton X-I 00, homogenate spread on agar plates, and enumerated for colony formation. S. aureus Newman (A) and its isogenic variants carrying transduced bursa aurealis insertions into isdA, isdB, isdC or isdH were analyzed. (G) Six week old BALB/c mice were infected by retroorbital injection with 5×10⁸ CFU S. aureus Newman or its isogenic variants and survival of BALB/c mice monitored over 10 days (240 hours).

FIG. 2. Generation of rabbit antibodies specific for iron-regulated surface determinants. (A) Schematic to illustrate the primary structure of the mature domain (lacking the N-terminal signal peptide and the C-terminal sorting signal) of IsdA, IsdB, IsdC and IsdH. NEAT domains are highlighted in grey and designated for each polypeptide. (B) Amino acid identity between the various NEAT domains of IsdA, IsdB, IsdC and IsdH is designated in percent of its sequence. (C) Recombinant, poly-histidine affinity tagged IsdA, IsdB, IsdC and IsdH were purified by affinity chromatography and analyzed by Coomassie-stained SDS-PAGE (A) or by immunoblot with rabbit antisera raised against purified IsdA (αIsdA), IsdB (αIsdB), IsdC (αIsdC), or IsdH (αIsdH). (D) Cross-reactivity of rabbit antibodies directed against IsdA, IsdB and IsdC was quantified by ELISA using purified antigen. (E and F) Antibodies directed against IsdA and IsdB were purified by affinity chromatography on a matrix with covalently linked IsdA or IsdB. Cross-reactive antibodies in the eluate were removed by chromatography over the reciprocal column (IsdA* and IsdB*) and analyzed by immunoblotting (E) or ELISA (F).

FIG. 3. Purified rabbit antibodies specific for iron-regulated surface determinants do not promote opsonophagocytic killing of S. aureus Newman in anti-coagulated blood from naive mice. Blood of BALB/c mice was drawn by cardiac puncture, treated with lepirudin, pooled and 0.5 ml aliquots infected with 1×10⁵ CFU of S. aureus Newman, which was derived from midlog cultures grown in TSB and washed with PBS. Staphylococci and blood were incubated with rotation at 37° C. Aliquots were removed at timed intervals (0, 15, 30 and 60 min), blood cells lysed with saponin and staphylococcal load enumerated by plating on agar and colony formation. Bacterial survival at indicated time intervals was calculated from the average of three experimental determinations, divided by the average staphylococcal load at the beginning of the experiment (time 0 min) and standard error of the means calculated.

FIG. 4. Purified rabbit antibodies specific for IsdA or IsdB protect mice against staphylococcal abscess formation and lethal challenge. (A) Affinity purified rabbit IgG (85 μg or 5 mg kg⁻¹ body weight) directed against IsdA or IsdB was injected into the peritoneal cavity of BALB/c mice. Twenty-four hours later, five animals were bled by cardiac puncture and serum IgG antibody levels against IsdA or IsdB were determined by ELISA. (B) Twenty-four hours following passive immunization, cohorts of mice were challenged with 1×10⁷ CFU S. aureus Newman via retro-orbital injection. After four days, animals were killed, kidneys removed and analyzed for histopathology (B) or staphylococcal load (C) as described in the legend to FIG. 1. Staphylococcal abscess communities at the center of these lesions are marked with blue arrows. Necrotic immune cells are marked with white arrows. (D) Twenty-four hours following passive immunization, cohorts of mice were challenged with 5×10⁸ CFU S. aureus Newman via retro-orbital injection. The development of lethal infections was monitored over the next 240 hours (ten days).

FIG. 5. Antibodies against IsdA and IsdB interfere with heme-iron scavenging of staphylococci. (A) Schematic illustrating the primary structure of IsdB_{N} (SEQ ID NO:128) and IsdB_{C} (SEQ ID NO:252), the approximate two halves of IsdB encompassing its hemoglobin-binding (IsdB_{N}) and heme transfer (IsdB_{C}) domains, respectively. (B) Purified hemoglobin (Hb) was incubated with glutathione S-transferase (GST) or its fusions to IsdB_{N}(GST-IsdB_{N}) and IsdB_{C} (GST-IsdB_{C}) and possible association between polypeptides detected by separating affinity chromatography eluates on Coomassie-stained SDS-PAGE. (C) Association of purified poly-histidine tagged IsdA, IsdB, IsdB_{N} or IsdB_{C} with Hb was measured by surface plasmon resonance in three experimental determinations; data generated for IsdB were used as calibration (100%). Average data and standard error of the means were recorded. Association of IsdB_{N} with Hb was perturbed with irrelevant IgG antibodies (anti-V10) or with anti-IsdB. (D) The ability of poly-histidine tagged IsdA, IsdC, IsdB_{N} or IsdB_{C} to bind hemin was measured as sample absorbance at 410 nm (Soret). Data generated for IsdB_{C} were used as calibration (100%). Association of IsdB_{C} with hemin was perturbed with irrelevant IgG antibodies (anti-V10) or with anti-IsdB.

FIG. 6. Antibodies against IsdB_{N} or IsdB_{C} protect mice against staphylococcal abscess formation and lethal challenge. (A) Affinity purified rabbit IgG (85 μg or 5 mg kg⁻¹ body weight) directed against IsdB_{N} or IsdB_{C} was injected into the peritoneal cavity of BALB/c mice. Twenty-four hours later, five animals were bled by cardiac puncture and serum IgG antibody levels against IsdB_{N} or IsdB_{C} were determined by ELISA. (B) Twenty-four hours following passive immunization, cohorts of mice were challenged with 1×10⁷ CFU S. aureus Newman via retro-orbital injection. After four days, animals were killed, kidneys removed and analyzed for histopathology (B) or staphylococcal load (C) as described in the legend to FIG. 1. Staphylococcal abscess communities at the center of these lesions are marked with blue arrows. Necrotic immune cells are marked with white arrows. (D) Twenty-four hours following passive immunization, cohorts of mice were challenged with 5×10⁸ CFU S. aureus Newman via retro-orbital injection. The development of lethal infections was monitored over the next 240 hours (ten days).

FIG. 7. Schematic diagram of primary structure of IsdA mutant proteins used to map MAb binding. Schematic illustrating the primary structure of poly-histidine variants of IsdA (IsdA-1_{FL50-311}, IsdA-2_{Δ50-89}, IsdA-3_{Δ90-29}, IsdA-4_{Δ130-169}, IsdA-5_{Δ170-209}, IsdA-6_{Δ210-249}, IsdA-7_{Δ250-311}). Variants were designed to contain consecutive deletions of 40 amino acids each and were cloned and purified by affinity chromatography. Regions in grey, amino acids 69-180, represent the NEAT domain (NEAr Transport), responsible for heme binding.

FIG. 8A-8C. Isd MAb Ig sequencing and alignment. Following RNA isolation and cDNA synthesis by RT-PCR, positive sequences were analyzed and V-(D)-J segments aligned via IMGT vquest. A. Isd MAbs 1B8 and 7D4 shared the same V,D and J sequences for the V_{H} genes. B. Isd MAbs 3D8, 7E9, 4H7 and 2A9 shared the same V and J sequences for the V_{L} genes and C. the third group included 5H8 and 3H11 which shared the same V and J sequences for their respective V_{L} genes.

FIG. 9A-9C. Alignment of translated nucleotide sequence for Isd MAbs.

FIG. 10A-E. Alignments of regions of IsdA (SEQ ID NO:1) and IsdB (SEQ ID NO:2) antigens. A. Alignment corresponding to deleted region from IsdA-2 (aa 50-89 of IsdA, FIG. 7). B. Alignment corresponding to deleted region from IsdA-4 (aa 130-169 of IsdA, FIG. 7). C. Alignment corresponding to deleted region from IsdA-5 (aa 170-209 of IsdA, FIG. 7). D. Alignment corresponding to deleted region from IsdA-6 (aa 210-249 of IsdA, FIG. 7). E. Alignment corresponding to deleted region from IsdA-7 (aa 250-311 of IsdA, FIG. 7).

FIG. 11A-B. Alignments of VL (FIG. 11A) and VH (FIG. 11B) CDR sequences of the indicated IsdA and IsdB binding antibodies of the embodiments. Sequences for the for the VL of 2A9 and the VH of 1B9 represent the alternative sequence for the immunoglobulin chains provided herein.

FIG. 12A-B. Alignments of VL (FIG. 11A) and VH (FIG. 11B) CDR sequences of the indicated antibodies of the embodiments. Left column indicates the IsdA/B binding specificity of the indicated antibody. Sequences for the for the alternative VL of 2A9 is indicated.

FIG. 13. Passive immunization experiments with 3D8 antibody. Bacterial load (CFU) was assessed in mice 5 days after infection with wt (Newman) or isdB-bacteria in the presence (abB) or absence of 3D8 antibody preadministration.

FIG. 14. Survival of iron starved S. aureus in human blood in the presence of IsdA or IsdB specific MAbs. S. aureus Newman was grown in chelex treated RPMI plus 0.2 mM 2′-2-dipyridyl to induce surface expression of IsdA and IsdB. 5×10⁶ CFU staphylococci, washed and resuspended in PBS, were incubated with 1 ml of lepirudin-treated human blood and of 2 μg·ml⁻¹ individual IsdA or IsdB MAbs or isotype controls with gentle rotation at 37° C. for 120 min. Blood cells were lysed with saponin and staphylococcal load enumerated by plating on agar to determine CFU. Experimental N=1, error bars represent standard deviation in bacterial counts for each sample. Bacterial survival at indicated time intervals was calculated from individual CFU inputs at time t=0 with percent calculated relative to no antibody control.

FIG. 15. IsdA and IsdB MAbs interfere with staphylococcal growth when human hemoglobin is the sole iron source. S. aureus Newman was iron starved by multiple rounds of growth in chelex treated RPMI plus 0.2 mM 2′-2-dipyridyl. These same cells were grown in the presence or absence of hemoglobin and 20 μg of the individual IsdA or IsdB MAbs. Growth was monitored at A₆₆₀ and heme content tracked by monitoring A₄₁₀ every 10 min. over 16 hours. Growth curves were normalized to media plus hemoglobin alone and peak absorbance with standard deviation plotted. Statistical significance was determined using the Kruskal Wallis analysis of variance with Prism graphpad software. Data is representative of an N=3 experiments, with each data point representing triplicate samples per experiment.

### DETAILED DESCRIPTION OF THE INVENTION

The inventors demonstrate that two iron-regulated surface determinants, IsdA and IsdB, are required for the pathogenesis of kidney abscess formation and lethal disease caused by S. aureus Newman. Affinity purified rabbit antibodies directed against either IsdA or IsdB generate significant protection against S. aureus abscess formation or lethal challenge, two diseases that involve intravenous inoculation of virulent staphylococci. Pathogenic processes that underlie both diseases occur over a period of two-to-four days in animal models.

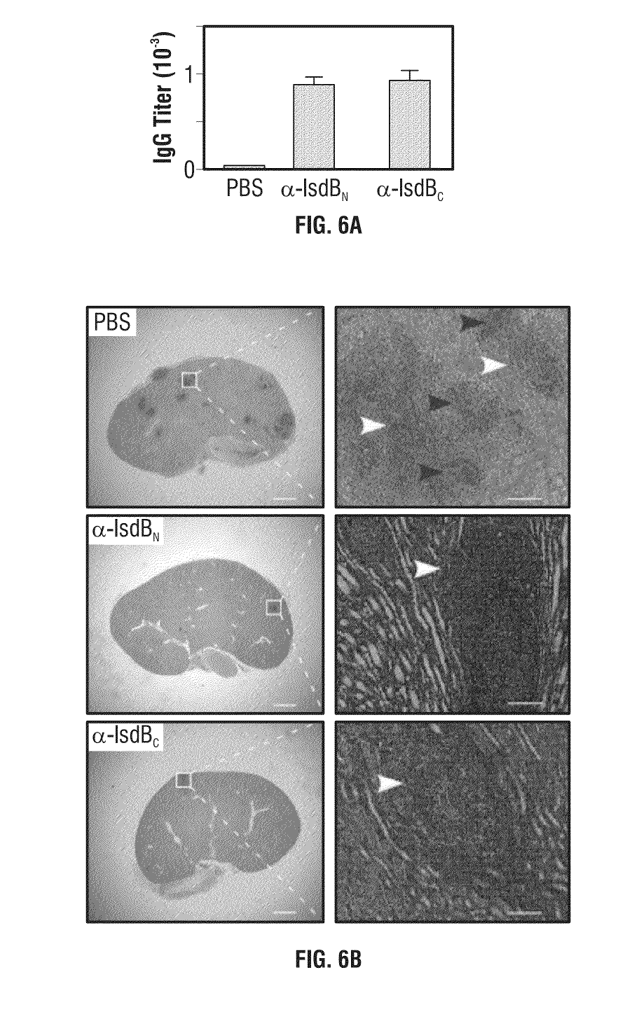

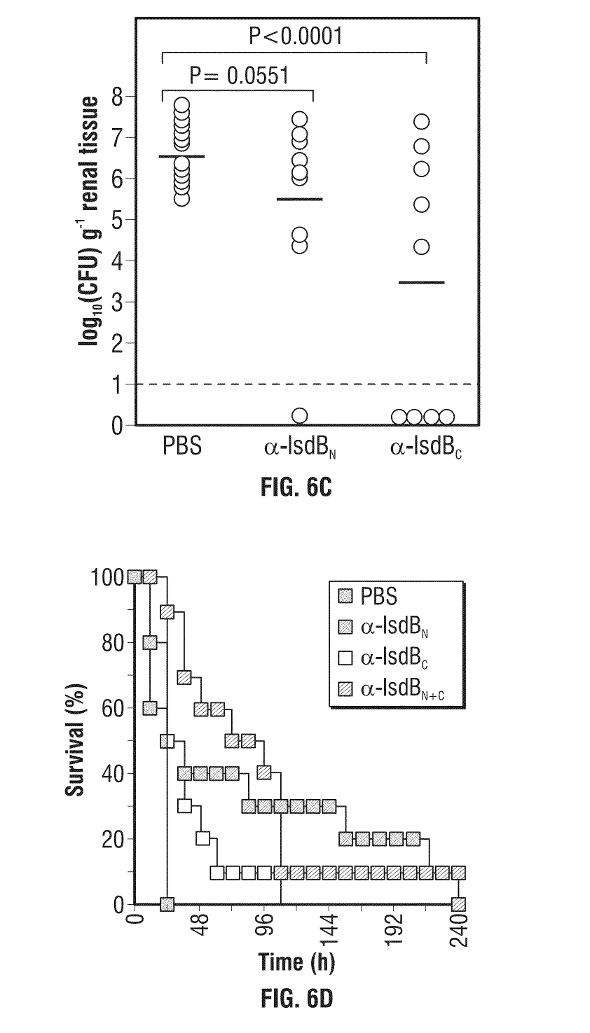

Purified rabbit antibodies hindered the ability of IsdB to bind hemoglobin or capture heme for subsequent heme-iron scavenging via a transport cascade in the staphylococcal envelope involving IsdA, IsdC as well as IsdE/IsdF (Mazmanian et al., 2003; Muryoi et al., 2008), and culminating in cleavage of the tetrapyrrol by IsdG/IsdI and the release of iron (Skaar et al., 2004). IsdB is currently being explored as a vaccine antigen to prevent staphylococcal infection of humans (Kuklin et al., 2006; Raedler et al., 2009). If so, the development of assays that monitor the attributes of IsdB-specific antibodies in blocking heme-iron transport could be considered as a correlate for immunity in humans. FIG. 6 indicates that antibodies directed against the IsdB_{N} (IsdB1) and IsdB_{C} (IsdB2) halves of the protein generate protection against staphylococcal abscess formation and lethal challenge. Combining antibodies specific for IsdB_{N} and IsdB_{C} clearly increased protection against lethal challenge. In certain aspects peptide segments or epitopes can be used to generate IsdA or IsdB antibodies.

### I. Iron Scavenging Mechanism of S. aureus

Staphylococci rely on surface protein mediated-adhesion to host cells or invasion of tissues as a strategy for escape from immune defenses. Furthermore, S. aureus utilize surface proteins to sequester iron from the host during infection. The majority of surface proteins involved in staphylococcal pathogenesis carry C-terminal sorting signals, i.e., they are covalently linked to the cell wall by sortases.

IsdA, a sortase A-anchored NEAT domain protein, transfers heme from the two hemophores IsdB and IsdH to IsdC, the central conduit of staphylococcal heme-iron scavenging. IsdC is anchored to the cell wall by sortase B and its unique position in the envelope enables the transfer of heme to IsdEF for import into the bacterial cytoplasm. Active immunization with IsdA antigen (Stranger-Jones et al., 2006) and, as demonstrated here, passive transfer of antibodies against IsdA provide experimental mice with a significant level of protection against staphylococcal abscess formation and lethal challenge. When compared with IsdB, antibodies against IsdA performed equally well in the renal abscess model. In contrast, IsdA antibodies did not achieve the same level of protection as IsdB antibodies when tested in the lethal challenge model. It is not clear that IsdC is accessible to antibodies on the staphylococcal surface (Marraffini and Schneewind, 2005; Mazmanian et al., 2003). Nevertheless, earlier work revealed a significant level of protection in mice following active immunization with IsdC, albeit that protection from abscess formation was not as high as measured for IsdA and IsdB (Stranger-Jones et al., 2006). Other sortase substrate polypeptides include, but are not limited to the amino acid sequence of SdrC, SdrD, SdrE, SpA, ClfA, ClfB, IsdC or SasF proteins from bacteria in the Staphylococcus genus.

Examples of various proteins that can be used in the context of the present invention can be identified by analysis of database submissions of bacterial genomes, including but not limited to accession numbers NC_{—}002951 (GI:57650036 and GenBank CP000046), NC_{—}002758 (GI:57634611 and GenBank BA000017), NC_{—}002745 (GI:29165615 and GenBank BA000018), NC_{—}003923 (GI:21281729 and GenBank BA000033), NC_{—}002952 (GI:49482253 and GenBank BX571856), NC_{—}002953 (GI:49484912 and GenBank BX571857), NC_{—}007793 (GI:87125858 and GenBank CP000255), NC_{—}007795 (GI:87201381 and GenBank CP000253) each of which are incorporated by reference.

The ‘isdA’ antigen is annotated as ‘IsdA protein’. In the NCTC 8325 strain isdA is SAOUHSC_{—}01081 (GI:88194829). In the Newman strain it is nwmn_{—}1041 (GI:151221253). Useful isdA antigens can elicit an antibody response (e.g. when administered to a human), and includes variants and fragments.

The ‘isdB’ antigen is annotated as ‘neurofilament protein isdB’. In the NCTC 8325 strain isdB is SAOUHSC_{—}01079 (GI:88194828). Useful isdB antigens can elicit an antibody response (e.g. when administered to a human), and includes fragments and variants.

The ‘clfA’ antigen is annotated as ‘clumping factor A’. In the NCTC 8325 strain clfA is SAOUHSC_{—}00812 (GI:88194572). In the Newman strain it is nwmn_{—}0756 (GI:151220968). Useful clfA antigens can elicit an antibody response (e.g. when administered to a human), and include variants and fragments.

The ‘clfB’ antigen is annotated as ‘clumping factor B’. In the NCTC 8325 strain clfB is SAOUHSC_{—}02963 (GI:88196585). In the Newman strain it is nwmn_{—}2529 (GI:151222741). Useful clfB antigens can elicit an antibody response (e.g. when administered to a human), and include variants and fragments.

The ‘eap’ antigen is annotated as ‘MHC class II analog protein’. In the NCTC 8325 strain eap is SAOUHSC_{—}02161 (GI:88195840). In the Newman strain it is nwmn_{—}1872 (GI:151222084). Useful eap antigens can elicit an antibody response (e.g. when administered to a human), and include variants and fragments.

The ‘ebhA’ antigen is annotated as ‘EbhA’. In the NCTC 8325 strain ebhA is SAOUHSC_{—}01447 and has amino acid sequence (GI:88195168). Useful ebhA antigens can elicit an antibody response (e.g. when administered to a human), and include variants and fragment.

The ‘emp’ antigen is annotated as ‘extracellular matrix and plasma binding protein’. In the NCTC 8325 strain emp is SAOUHSC_{—}00816 (GI:88194575). In the Newman strain it is nwmn_{—}0758 (GI:151220970). Useful emp antigens can elicit an antibody response (e.g., when administered to a human), and include variants and fragments.

The ‘esxA’ antigen is annotated as ‘protein’. In the NCTC 8325 strain esxA is SAOUHSC_{—}00257 (GI:88194063). Useful esxA antigens can elicit an antibody response (e.g. when administered to a human), and include variants and fragments.

The ‘esxB’ antigen is annotated as ‘esxB’. In the NCTC 8325 strain esxB is SAOUHSC_{—}00265 (GI:88194070). Useful esxB antigens can elicit an antibody response (e.g. when administered to a human), and include variants and fragments.

The ‘Hla’ antigen is the ‘alpha-hemolysin precursor’ also known as ‘alpha toxin’ or simply ‘hemolysin’. In the Newman strain it is nwmn_{—}1073 (GI:151221285). Hla is an important virulence determinant produced by most strains of S. aureus, having pore-forming and hemolytic activity. Anti-Hla antibodies can neutralize the detrimental effects of the toxin in animal models. Useful Hla antigens can elicit an antibody response (e.g., when administered to a human), and include variants and fragments. Hla's toxicity can be avoided in compositions of the invention by chemical inactivation (e.g. using formaldehyde, glutaraldehyde or other cross-linking reagents). Instead, however, it is preferred to use mutant forms of Hla which remove its toxic activity while retaining its immunogenicity. Such detoxified mutants are already known in the art, including Hla-H35L.

The ‘isdC’ antigen is annotated as ‘protein’. In the NCTC 8325 strain isdC is SAOUHSC_{—}01082 (GI:88194830). Useful isdC antigens can elicit an antibody response (e.g. when administered to a human), and fragments and variants.

The ‘sasF’ antigen is annotated as ‘sasF protein’. In the NCTC 8325 strain sasF is SAOUHSC_{—}02982 (GI:88196601). Useful sasF antigens can elicit an antibody response (e.g. when administered to a human), and fragments and variants.

The ‘sdrC’ antigen is annotated as ‘sdrC protein’. In the NCTC 8325 strain sdrC is SAOUHSC_{—}00544 and has amino acid sequence (GI:88194324). Useful sdrC antigens can elicit an antibody response (e.g. when administered to a human), and fragments and variants.

The ‘sdrD’ antigen is annotated as ‘sdrD protein’. In the NCTC 8325 strain sdrD is SAOUHSC_{—}00545 (GI:88194325). Useful sdrD antigens can elicit an antibody response (e.g. when administered to a human), and fragments and variants.

The ‘sdrE2’ antigen is annotated as ‘Ser-Asp rich fibrinogen/bone sialoprotein-binding protein SdrE’. In the Newman strain sdrE2 is NWMN_{—}0525 (GI:151220737). Useful sdrE2 antigens can elicit an antibody response (e.g. when administered to a human), and includes fragments and variants.

The ‘spa’ antigen is annotated as ‘protein A’ or ‘SpA’. All Staphylococcus aureus strains express the structural gene for spa, a well characterized virulence factor whose cell wall anchored surface protein product (SpA) encompasses five highly homologous immunoglobulin binding domains designated E, D, A, B, and C (Sjodahl, 1977). These domains display ˜80% identity at the amino acid level, are 56 to 61 residues in length, and are organized as tandem repeats (Uhlen et al., 1984). SpA is synthesized as a precursor protein with an N-terminal YSIRK/GS signal peptide and a C-terminal LPXTG motif sorting signal (DeDent et al., 2008; Schneewind et al., 1992). Cell wall anchored Protein A is displayed in great abundance on the staphylococcal surface (DeDent et al., 2007; Sjoquist et al., 1972). Each of its immunoglobulin binding domains is composed of anti-parallel α-helices that assemble into a three helix bundle and bind the Fc domain of immunoglobulin G (IgG) (Deisenhofer, 1981; Deisenhofer et al., 1978), the VH3 heavy chain (Fab) of IgM (i.e., the B cell receptor) (Graille et al., 2000), the von Willebrand factor at its A1 domain [vWF AI is a ligand for platelets] (O'Seaghdha et al., 2006) and the tumor necrosis factor α (TNF-α) receptor I (TNFRI) (Gomez et al., 2006), which is displayed on surfaces of airway epithelia (Gomez et al., 2004).

In the NCTC 8325 strain spa is SAOUHSC_{—}00069 (GI:88193885). In the Newman strain it is nwmn_{—}0055 (GI:151220267). Useful spa antigens can elicit an antibody response (e.g. when administered to a human), and includes variants and fragments. Useful spa antigens include SpA variants comprising a variant A, B, C, D and E domain. Useful spa antigens also include SpA segments and SpA variants comprising a segment of SpA. The SpA segment can comprise at least or at most 1, 2, 3, 4, 5 or more IgG binding domains. The IgG domains can be at least or at most 1, 2, 3, 4, 5 or more variant A, B, C, D or E domains. Useful spa antigens also include SpA variants comprising a variant A domain, a variant B domain, a variant C domain, a variant D domain or a variant E domain.

In certain aspects a SpA variant includes a substitution of (a) one or more amino acid substitution in an IgG Fc binding sub-domain of SpA domain A, B, C, D and/or E that disrupts or decreases binding to IgG Fc, and (b) one or more amino acid substitution in a V_{H}3 binding sub-domain of SpA domain A, B, C, D, and/or E that disrupts or decreases binding to V_{H}3. In certain embodiments, a variant SpA comprises at least or at most 1, 2, 3, 4, 5, 6, 7, 8, 9, 10 or more variant SpA domain D peptides.

As used herein, a “protein” or “polypeptide” refers to a molecule comprising at least ten amino acid residues. In some embodiments, a wild-type version of a protein or polypeptide are employed, however, in many embodiments of the invention, a modified protein or polypeptide is employed to generate an immune response. The terms described above may be used interchangeably. A “modified protein” or “modified polypeptide” refers to a protein or polypeptide whose chemical structure, particularly its amino acid sequence, is altered with respect to the wild-type protein or polypeptide. In some embodiments, a modified protein or polypeptide has at least one modified activity or function (recognizing that proteins or polypeptides may have multiple activities or functions). It is specifically contemplated that a modified protein or polypeptide may be altered with respect to one activity or function yet retain a wild-type activity or function in other respects, such as immunogenicity.

As used herein, an “amino molecule” refers to any amino acid, amino acid derivative, or amino acid mimic known in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

Accordingly, the term “proteinaceous composition” encompasses amino molecule sequences comprising at least one of the 20 common amino acids in naturally synthesized proteins, or at least one modified or unusual amino acid.

Proteinaceous compositions may be made by any technique known to those of skill in the art, including (i) the expression of proteins, polypeptides, or peptides through standard molecular biological techniques, (ii) the isolation of proteinaceous compounds from natural sources, or (iii) the chemical synthesis of proteinaceous materials. The nucleotide as well as the protein, polypeptide, and peptide sequences for various genes have been previously disclosed, and may be found in the recognized computerized databases. One such database is the National Center for Biotechnology Information's GenBank and GenPept databases (on the World Wide Web at ncbi.nlm.nih.gov/). The coding regions for these genes may be amplified and/or expressed using the techniques disclosed herein or as would be known to those of ordinary skill in the art.

### II. Proteinaceous Compositions

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins of the invention may be recombinant, or synthesized in vitro. Alternatively, a non-recombinant or recombinant protein may be isolated from bacteria. It is also contemplated that a bacteria containing such a variant may be implemented in compositions and methods of the invention. Consequently, a protein need not be isolated.

The term “functionally equivalent codon” is used herein to refer to codons that encode the same amino acid, such as the six codons for arginine or serine, and also refers to codons that encode biologically equivalent amino acids (see Table 1, below).

Codon Table

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

It also will be understood that amino acid and nucleic acid sequences may include additional residues, such as additional N- or C-terminal amino acids, or 5′ or 3′ sequences, respectively, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. It is understood that an amino acid can be substituted for another having a similar hydrophilicity value and still produce a biologically equivalent and immunologically equivalent protein.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known and include: arginine and lysine; glutamate and aspartate; serine and threonine; glutamine and asparagine; and valine, leucine and isoleucine.

It is contemplated that in compositions of the invention, there is between about 0.001 mg and about 10 mg of total polypeptide, peptide, and/or protein per ml. Thus, the concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein). Of this, about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% may be an antibody that binds IsdA and/or IsdB, and may be used in combination with other staphylococcal proteins described herein.

A. Polypeptides and Polypeptide Production

The present invention describes polypeptides, peptides, and proteins and immunogenic fragments thereof for use in various embodiments of the present invention. For example, specific antibodies are assayed for or used in neutralizing or inhibiting Staphylococcal infection. In specific embodiments, all or part of the proteins of the invention can also be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Alternatively, recombinant DNA technology may be employed wherein a nucleotide sequence which encodes a peptide or polypeptide of the invention is inserted into an expression vector, transformed or transfected into an appropriate host cell and cultivated under conditions suitable for expression.

One embodiment of the invention includes the use of gene transfer to cells, including microorganisms, for the production and/or presentation of proteins. The gene for the protein of interest may be transferred into appropriate host cells followed by culture of cells under the appropriate conditions. A nucleic acid encoding virtually any polypeptide may be employed. The generation of recombinant expression vectors, and the elements included therein, are discussed herein. Alternatively, the protein to be produced may be an endogenous protein normally synthesized by the cell used for protein production.

In a certain aspects an immunogenic IsdA and/or IsdB fragments according to the invention comprises substantially all of the extracellular domain of a protein which has at least 85% identity, at least 90% identity, at least 95% identity, or at least 97-99% identity, including all values and ranges there between, to a sequence selected over the length of the fragment sequence.

Also included in immunogenic compositions of the invention are fusion proteins composed of Staphylococcal proteins, or immunogenic fragments of staphylococcal proteins (e.g., IsdA and/or IsdB or consensus peptides thereof). Alternatively, the invention also includes individual fusion proteins of Staphylococcal proteins or immunogenic fragments thereof, as a fusion protein with heterologous sequences such as a provider of T-cell epitopes or purification tags, for example: β-galactosidase, glutathione-S-transferase, green fluorescent proteins (GFP), epitope tags such as FLAG, myc tag, poly histidine, or viral surface proteins such as influenza virus haemagglutinin, or bacterial proteins such as tetanus toxoid, diphtheria toxoid, CRM197.

B. Antibodies and Antibody-Like Molecules

In certain aspects of the invention, one or more antibodies or antibody-like molecules (e.g., polypeptides comprising antibody CDR domains) may be obtained or produced which have a specificity for an IsdA and/or IsdB. These antibodies may be used in various diagnostic or therapeutic applications described herein.

As used herein, the term “antibody” is intended to refer broadly to any immunologic binding agent such as IgG, IgM, IgA, IgD and IgE as well as polypeptides comprising antibody CDR domains that retain antigen binding activity. Thus, the term “antibody” is used to refer to any antibody-like molecule that has an antigen binding region, and includes antibody fragments such as Fab′, Fab, F(ab′)₂, single domain antibodies (DABs), Fv, scFv (single chain Fv), and polypeptides with antibody CDRs, scaffolding domains that display the CDRs (e.g., anticalins) or a nanobody. For example, the antibody can be a VHH (i.e., an antigen-specific VHH) antibody that comprises only a heavy chain. For example, such antibody molecules can be derived from a llama or other camelid antibody (e.g., a camelid IgG2 or IgG3, or a CDR-displaying frame from such camelid Ig) or from a shark antibody. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

“Mini-antibodies” or “minibodies” are also contemplated for use with the present invention. Minibodies are sFv polypeptide chains which include oligomerization domains at their C-termini, separated from the sFv by a hinge region. Pack et al. (1992) Biochem 31:1579-1584. The oligomerization domain comprises self-associating α-helices, e.g., leucine zippers, that can be further stabilized by additional disulfide bonds. The oligomerization domain is designed to be compatible with vectorial folding across a membrane, a process thought to facilitate in vivo folding of the polypeptide into a functional binding protein. Generally, minibodies are produced using recombinant methods well known in the art. See, e.g., Pack et al. (1992) Biochem 31:1579-1584; Cumber et al. (1992) J Immunology 149B:120-126.

Antibody-like binding peptidomimetics are also contemplated in the present invention. Liu et al. Cell Mol Biol (Noisy-le-grand). 2003 March; 49(2):209-16 describe “antibody like binding peptidomimetics” (ABiPs), which are peptides that act as pared-down antibodies and have certain advantages of longer serum half-life as well as less cumbersome synthesis methods. Likewise, in some aspects, antibody-like molecules are cyclic or bicyclic peptides. For example, methods for isolating antigen-binding cyclic peptides (e.g., by phage display) and for using the such peptides are provided in U.S. Patent Publn. No. 20100317547, incorporated herein by reference. In some embodiments, a scaffolding polypeptide can be a “molecular affinity clamp” see, for example, U.S. Patent Publn. Nos. 20110143963 and 20110045604, incorporated herein by reference.

Alternative scaffolds for antigen binding peptides, such as CDRs are also available and can be used to generate IsdA and/or IsdB-binding molecules in accordance with the embodiments. Generally, a person skilled in the art knows how to determine the type of protein scaffold on which to graft at least one of the CDRs arising from the original antibody. More particularly, it is known that to be selected such scaffolds must meet the greatest number of criteria as follows (Skerra A., J. Mol. Recogn., 2000, 13:167-187): good phylogenetic conservation; known three-dimensional structure (as, for example, by crystallography, NMR spectroscopy or any other technique known to a person skilled in the art); small size; few or no post-transcriptional modifications; and/or easy to produce, express and purify.

The origin of such protein scaffolds can be, but is not limited to, the structures selected among: fibronectin (see, e.g., U.S. Patent Publn. No. 20090253899, incorporated herein by reference) and preferentially fibronectin type III domain 10, lipocalin, anticalin (Skerra A., J. Biotechnol., 2001, 74(4):257-75), protein Z arising from domain B of protein A of Staphylococcus aureus, thioredoxin A or proteins with a repeated motif such as the “ankyrin repeat” (Kohl et al., PNAS, 2003, vol. 100, No. 4, 1700-1705), the “armadillo repeat”, the “leucine-rich repeat” and the “tetratricopeptide repeat”. For example, anticalins or lipocalin derivatives are a type of binding proteins that have affinities and specificities for various target molecules and can be used as IsdA and/or IsdB binding molecules. Such proteins are described in US Patent Publication Nos. 20100285564, 20060058510, 20060088908, 20050106660, and PCT Publication No. WO2006/056464, incorporated herein by reference.

Scaffolds derived from toxins such as, for example, toxins from scorpions, insects, plants, mollusks, etc., and the protein inhibiters of neuronal NO synthase (PIN) may also be used in certain aspects.

Monoclonal antibodies (MAbs) are recognized to have certain advantages, e.g., reproducibility and large-scale production. The invention provides monoclonal antibodies of the human, murine, monkey, rat, hamster, rabbit and chicken origin.

“Humanized” antibodies are also contemplated, as are chimeric antibodies from mouse, rat, or other species, bearing human constant and/or variable region domains, bispecific antibodies, recombinant and engineered antibodies and fragments thereof. As used herein, the term “humanized” immunoglobulin refers to an immunoglobulin comprising a human framework region and one or more CDR's from a non-human (usually a mouse or rat) immunoglobulin. The non-human immunoglobulin providing the CDR's is called the “donor” and the human immunoglobulin providing the framework is called the “acceptor”. A “humanized antibody” is an antibody comprising a humanized light chain and a humanized heavy chain immunoglobulin.

1. Methods for Generating Antibodies

Methods for generating antibodies (e.g., monoclonal antibodies and/or monoclonal antibodies) are known in the art. Briefly, a polyclonal antibody is prepared by immunizing an animal with an IsdA and/or IsdB polypeptide or a portion thereof in accordance with the present invention and collecting antisera from that immunized animal.

A wide range of animal species can be used for the production of antisera. Typically the animal used for production of antisera is a rabbit, a mouse, a rat, a hamster, a guinea pig or a goat. The choice of animal may be decided upon the ease of manipulation, costs or the desired amount of sera, as would be known to one of skill in the art. It will be appreciated that antibodies of the invention can also be produced transgenically through the generation of a mammal or plant that is transgenic for the immunoglobulin heavy and light chain sequences of interest and production of the antibody in a recoverable form therefrom. In connection with the transgenic production in mammals, antibodies can be produced in, and recovered from, the milk of goats, cows, or other mammals. See, e.g., U.S. Pat. Nos. 5,827,690, 5,756,687, 5,750,172, and 5,741,957.

As is also well known in the art, the immunogenicity of a particular immunogen composition can be enhanced by the use of non-specific stimulators of the immune response, known as adjuvants. Suitable adjuvants include any acceptable immunostimulatory compound, such as cytokines, chemokines, cofactors, toxins, plasmodia, synthetic compositions or vectors encoding such adjuvants.

Adjuvants that may be used in accordance with the present invention include, but are not limited to, IL-1, IL-2, IL-4, IL-7, IL-12, -interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion is also contemplated. MHC antigens may even be used. Exemplary adjuvants may include complete Freund's adjuvant (a non-specific stimulator of the immune response containing killed Mycobacterium tuberculosis), incomplete Freund's adjuvants and/or aluminum hydroxide adjuvant.

In addition to adjuvants, it may be desirable to coadminister biologic response modifiers (BRM), which have been shown to upregulate T cell immunity or downregulate suppressor cell activity. Such BRMs include, but are not limited to, Cimetidine (CIM; 1200 mg/d) (Smith/Kline, PA); low-dose Cyclophosphamide (CYP; 300 mg/m2) (Johnson/Mead, NJ), cytokines such as -interferon, IL-2, or IL-12 or genes encoding proteins involved in immune helper functions, such as B-7.

The amount of immunogen composition used in the production of antibodies varies upon the nature of the immunogen as well as the animal used for immunization. A variety of routes can be used to administer the immunogen including but not limited to subcutaneous, intramuscular, intradermal, intraepidermal, intravenous and intraperitoneal: The production of antibodies may be monitored by sampling blood of the immunized animal at various points following immunization.

A second, booster dose (e.g., provided in an injection), may also be given. The process of boosting and titering is repeated until a suitable titer is achieved. When a desired level of immunogenicity is obtained, the immunized animal can be bled and the serum isolated and stored, and/or the animal can be used to generate MAbs.

For production of rabbit polyclonal antibodies, the animal can be bled through an ear vein or alternatively by cardiac puncture. The removed blood is allowed to coagulate and then centrifuged to separate serum components from whole cells and blood clots. The serum may be used as is for various applications or else the desired antibody fraction may be purified by well-known methods, such as affinity chromatography using another antibody, a peptide bound to a solid matrix, or by using, e.g., protein A or protein G chromatography, among others.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified protein, polypeptide, peptide or domain, be it a wild-type or mutant composition. The immunizing composition is administered in a manner effective to stimulate antibody producing cells.

The methods for generating monoclonal antibodies (MAbs) generally begin along the same lines as those for preparing polyclonal antibodies. In some embodiments, Rodents such as mice and rats are used in generating monoclonal antibodies. In some embodiments, rabbit, sheep or frog cells are used in generating monoclonal antibodies. The use of rats is well known and may provide certain advantages (Goding, 1986, pp. 60 61). Mice (e.g., BALB/c mice) are routinely used and generally give a high percentage of stable fusions.

The animals are injected with antigen, generally as described above. The antigen may be mixed with adjuvant, such as Freund's complete or incomplete adjuvant. Booster administrations with the same antigen or DNA encoding the antigen may occur at approximately two-week intervals.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Generally, spleen cells are a rich source of antibody-producing cells that are in the dividing plasmablast stage. Typically, peripheral blood cells may be readily obtained, as peripheral blood is easily accessible.

In some embodiments, a panel of animals will have been immunized and the spleen of an animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody producing B lymphocytes from the immunized animal are then fused with cells of an immortal myeloma cell, generally one of the same species as the animal that was immunized. Myeloma cell lines suited for use in hybridoma producing fusion procedures preferably are non antibody producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65 66, 1986; Campbell, pp. 75 83, 1984). cites). For example, where the immunized animal is a mouse, one may use P3 X63/Ag8, X63 Ag8.653, NS1/1.Ag 4 1, Sp210 Ag14, FO, NSO/U, MPC 11, MPC11 X45 GTG 1.7 and S194/5XX0 Bul; for rats, one may use R210.RCY3, Y3 Ag 1.2.3, IR983F and 4B210; and U 266, GM1500 GRG2, LICR LON HMy2 and UC729 6 are all useful in connection with human cell fusions. See Yoo et al., J Immunol Methods. 2002 Mar. 1; 261(1-2):1-20, for a discussion of myeloma expression systems.

One murine myeloma cell is the NS-1 myeloma cell line (also termed P3-NS-1-Ag-4-1), which is readily available from the NIGMS Human Genetic Mutant Cell Repository by requesting cell line repository number GM3573. Another mouse myeloma cell line that may be used is the 8 azaguanine resistant mouse murine myeloma SP2/0 non producer cell line.

Methods for generating hybrids of antibody producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al., (1977). The use of electrically induced fusion methods is also appropriate (Goding pp. 7174, 1986).

Fusion procedures usually produce viable hybrids at low frequencies, about 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody producing cell lines, which clones can then be propagated indefinitely to provide MAbs. The cell lines may be exploited for MAb production in two basic ways. First, a sample of the hybridoma can be injected (often into the peritoneal cavity) into a histocompatible animal of the type that was used to provide the somatic and myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific monoclonal antibody produced by the fused cell hybrid. The body fluids of the animal, such as serum or ascites fluid, can then be tapped to provide MAbs in high concentration. Second, the individual cell lines could be cultured in vitro, where the MAbs are naturally secreted into the culture medium from which they can be readily obtained in high concentrations.

Further, expression of antibodies of the invention (or other moieties therefrom) from production cell lines can be enhanced using a number of known techniques. For example, the glutamine synthetase and DHFR gene expression systems are common approaches for enhancing expression under certain conditions. High expressing cell clones can be identified using conventional techniques, such as limited dilution cloning and Microdrop technology. The GS system is discussed in whole or part in connection with European Patent Nos. 0 216 846, 0 256 055, and 0 323 997 and European Patent Application No. 89303964.4.

MAbs produced by either means may be further purified, if desired, using filtration, centrifugation and various chromatographic methods such as HPLC or affinity chromatography. Fragments of the monoclonal antibodies of the invention can be obtained from the monoclonal antibodies so produced by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It is also contemplated that a molecular cloning approach may be used to generate monoclonal antibodies. In one embodiment, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 104 times as many antibodies can be produced and screened in a single round, and that new specificities are generated by H and L chain combination which further increases the chance of finding appropriate antibodies. Target-binding (e.g., IsdA and/or IsdB) single domain antibodies can also be isolated by use of display libraries, see for example, U.S. Patent Appln. No. 20110183863, incorporated herein by reference. Ribosome expression libraries for isolation of target-bind Ig coding sequences are also described in U.S. Patent Appln. No. 20040161748; 20070299246 and 20080293591, each incorporated herein by reference.

Another embodiment of the invention for producing antibodies according to the present invention is found in U.S. Pat. No. 6,091,001, which describes methods to produce a cell expressing an antibody from a genomic sequence of the cell comprising a modified immunoglobulin locus using Cre-mediated site-specific recombination is disclosed. The method involves first transfecting an antibody-producing cell with a homology-targeting vector comprising a lox site and a targeting sequence homologous to a first DNA sequence adjacent to the region of the immunoglobulin loci of the genomic sequence which is to be converted to a modified region, so the first lox site is inserted into the genomic sequence via site-specific homologous recombination. Then the cell is transfected with a lox-targeting vector comprising a second lox site suitable for Cre-mediated recombination with the integrated lox site and a modifying sequence to convert the region of the immunoglobulin loci to the modified region. This conversion is performed by interacting the lox sites with Cre in vivo, so that the modifying sequence inserts into the genomic sequence via Cre-mediated site-specific recombination of the lox sites.

Alternatively, monoclonal antibody fragments encompassed by the present invention can be synthesized using an automated peptide synthesizer, or by expression of full-length gene or of gene fragments in E. coli.

C. Antibody and Polypeptide Conjugates

The present invention provides antibodies and antibody-like molecules against IsdA and/or IsdB proteins, polypeptides and peptides that are linked to at least one agent to form an antibody conjugate or payload. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, therapeutic enzymes, antibiotics, radio-labeled nucleotides and the like. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to which they are attached to be detected, and/or further quantified if desired.

Antibody conjugates are generally preferred for use as diagnostic agents. Antibody diagnostics generally fall within two classes, those for use in in vitro diagnostics, such as in a variety of immunoassays, and/or those for use in vivo diagnostic protocols, generally known as “antibody directed imaging”. Many appropriate imaging agents are known in the art, as are methods for their attachment to antibodies (see, for e.g., U.S. Pat. Nos. 5,021,236; 4,938,948; and 4,472,509, each incorporated herein by reference). The imaging moieties used can be paramagnetic ions; radioactive isotopes; fluorochromes; NMR-detectable substances; X-ray imaging.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might use astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often used in certain embodiments, and technicium^{99m} and/or indium¹¹¹ are also often used due to their low energy and suitability for long range detection. Radioactively labeled monoclonal antibodies of the present invention may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the invention may be labeled with technetium99m by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red, among others.

Antibody conjugates contemplated in the present invention include those intended primarily for use in vitro, where the antibody is linked to a secondary binding ligand and/or to an enzyme (an enzyme tag) that will generate a colored product upon contact with a chromogenic substrate. Examples of suitable enzymes include, but are not limited to, urease, alkaline phosphatase, (horseradish) hydrogen peroxidase or glucose oxidase. Preferred secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; and Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6? -diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In some embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

In some embodiments of the invention, anti-IsdA and/or IsdB antibodies are linked to semiconductor nanocrystals such as those described in U.S. Pat. Nos. 6,048,616; 5,990,479; 5,690,807; 5,505,928; 5,262,357 (all of which are incorporated herein in their entireties); as well as PCT Publication No. 99/26299 (published May 27, 1999). In particular, exemplary materials for use as semiconductor nanocrystals in the biological and chemical assays of the present invention include, but are not limited to those described above, including group II-VI, III-V and group IV semiconductors such as ZnS, ZnSe, ZnTe, CdS, CdSe, CdTe, MgS, MgSe, MgTe, CaS, CaSe, CaTe, SrS, SrSe, SrTe, BaS, BaSe, BaTe, GaN, GaP, GaAs, GaSb, InP, InAs, InSb, AlS, AlP, AlSb, PbS, PbSe, Ge and Si and ternary and quaternary mixtures thereof. Methods for linking semiconductor nanocrystals to antibodies are described in U.S. Pat. Nos. 6,630,307 and 6,274,323.

### III. Nucleic Acids

In certain embodiments, the present invention concerns recombinant polynucleotides encoding the proteins, polypeptides, or peptides of the invention. Polynucleotide sequences contemplated include those encoding antibodies to IsdA and/or IsdB or consensus peptides, or peptides and epitopes of IsdA and/or IsdB.

As used in this application, the term “polynucleotide” refers to a nucleic acid molecule that either is recombinant or has been isolated free of total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein (see Table 1 above).

In particular embodiments, the invention concerns isolated nucleic acid segments and recombinant vectors incorporating nucleic acid sequences that encode an antibody or antibody fragment that binds IsdA and/or IsdB or a consensus peptide thereof, or encode a peptide, antigen, or an epitope of IsdA and/or IsdB or a consensus thereof. The term “recombinant” may be used in conjunction with a polypeptide or the name of a specific polypeptide, and this generally refers to a polypeptide produced from a nucleic acid molecule that has been manipulated in vitro or that is a replication product of such a molecule.

The nucleic acid segments used in the present invention, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

In certain embodiments, the present invention provides polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence of this invention using the methods described herein (e.g., BLAST analysis using standard parameters). In certain aspects, the isolated polynucleotide of the invention will comprise a nucleotide sequence encoding a polypeptide that has at least 90%, preferably 95% and above, identity to an amino acid sequence of the invention, over the entire length of the sequence; or a nucleotide sequence complementary to said isolated polynucleotide.

A. Vectors

Polypeptides of the invention may be encoded by a nucleic acid molecule. The nucleic acid molecule can be in the form of a nucleic acid vector. The term “vector” is used to refer to a carrier nucleic acid molecule into which a heterologous nucleic acid sequence can be inserted for introduction into a cell where it can be replicated and expressed. A nucleic acid sequence can be “heterologous,” which means that it is in a context foreign to the cell in which the vector is being introduced or to the nucleic acid in which is incorporated, which includes a sequence homologous to a sequence in the cell or nucleic acid but in a position within the host cell or nucleic acid where it is ordinarily not found. Vectors include DNAs, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example Sambrook et al., 2001; Ausubel et al., 1996, both incorporated herein by reference). Vectors of the invention may be used in a host cell to produce an antibody that binds IsdA and/or IsdB or a peptide or consensus peptide thereof.

The term “expression vector” refers to a vector containing a nucleic acid sequence coding for at least part of a gene product capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described herein.

A “promoter” is a control sequence. The promoter is typically a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and expression of that sequence. A promoter may or may not be used in conjunction with an “enhancer,” which refers to a cis-acting regulatory sequence involved in the transcriptional activation of a nucleic acid sequence.

The particular promoter that is employed to control the expression of peptide or protein encoding polynucleotide of the invention is not believed to be critical, so long as it is capable of expressing the polynucleotide in a targeted cell, preferably a bacterial cell. Where a human cell is targeted, it is preferable to position the polynucleotide coding region adjacent to and under the control of a promoter that is capable of being expressed in a human cell. Generally speaking, such a promoter might include either a bacterial, human or viral promoter.

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals.

Vectors can include a multiple cloning site (MCS), which is a nucleic acid region that contains multiple restriction enzyme sites, any of which can be used in conjunction with standard recombinant technology to digest the vector. (See Carbonelli et al., 1999, Levenson et al., 1998, and Cocea, 1997, incorporated herein by reference.)

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression. (See Chandler et al., 1997, incorporated herein by reference.)

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels. In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message.

In expression, particularly eukaryotic expression, one will typically include a polyadenylation signal to effect proper polyadenylation of the transcript.

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

B. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

C. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

In addition to the disclosed expression systems of the invention, other examples of expression systems include STRATAGENE®'s COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

D. Methods of Gene Transfer

Suitable methods for nucleic acid delivery to effect expression of compositions of the present invention are believed to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Through the application of techniques such as these, organelle(s), cell(s), tissue(s) or organism(s) may be stably or transiently transformed.

### IV. Methods of Treatment

As discussed above, the compositions and methods of using these compositions can treat a subject (e.g., limiting abscess persistence) having, suspected of having, or at risk of developing an infection or related disease, particularly those related to staphylococci. One use of the immunogenic compositions of the invention is to prevent nosocomial infections by inoculating a subject prior to hospital treatment.

As used herein the phrase “immune response” or its equivalent “immunological response” refers to a humoral (antibody mediated), cellular (mediated by antigen-specific T cells or their secretion products) or both humoral and cellular response directed against a protein, peptide, or polypeptide of the invention in a recipient patient. Treatment or therapy can be an active immune response induced by administration of immunogen or a passive therapy effected by administration of antibody, antibody containing material, or primed T-cells.

As used herein “passive immunity” refers to any immunity conferred upon a subject by administration of immune effectors including cellular mediators or protein mediators (e.g., monoclonal and/or polyclonal antibodies). A monoclonal or polyclonal antibody composition may be used in passive immunization for the prevention or treatment of infection by organisms that carry the antigen recognized by the antibody. An antibody composition may include antibodies that bind to a variety of antigens that may in turn be associated with various organisms. The antibody component can be a polyclonal antiserum. In certain aspects the antibody or antibodies are affinity purified from an animal or second subject that has been challenged with an antigen(s). Alternatively, an antibody mixture may be used, which is a mixture of monoclonal and/or polyclonal antibodies to antigens present in the same, related, or different microbes or organisms, such as gram-positive bacteria, gram-negative bacteria, including but not limited to staphylococcus bacteria.

Passive immunity may be imparted to a patient or subject by administering to the patient immunoglobulins (Ig) or fragments thereof and/or other immune factors obtained from a donor or other non-patient source having a known immunoreactivity. In other aspects, an antigenic composition of the present invention can be administered to a subject who then acts as a source or donor for globulin, produced in response to challenge from the composition (“hyperimmune globulin”), that contains antibodies directed against Staphylococcus or other organism. A subject thus treated would donate plasma from which hyperimmune globulin would then be obtained, via conventional plasma-fractionation methodology, and administered to another subject in order to impart resistance against or to treat staphylococcus infection. Hyperimmune globulins according to the invention are particularly useful for immune-compromised individuals, for individuals undergoing invasive procedures or where time does not permit the individual to produce their own antibodies in response to vaccination. See U.S. Pat. Nos. 6,936,258, 6,770,278, 6,756,361, 5,548,066, 5,512,282, 4,338,298, and 4,748,018, each of which is incorporated herein by reference in its entirety, for exemplary methods and compositions related to passive immunity.

For purposes of this specification and the accompanying claims the terms “epitope” and “antigenic determinant” are used interchangeably to refer to a site on an antigen to which B and/or T cells respond or recognize. B-cell epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include those methods described in Epitope Mapping Protocols (1996). T cells recognize continuous epitopes of about nine amino acids for CD8 cells or about 13-15 amino acids for CD4 cells. T cells that recognize the epitope can be identified by in vitro assays that measure antigen-dependent proliferation, as determined by ³H-thymidine incorporation by primed T cells in response to an epitope (Burke et al., 1994), by antigen-dependent killing (cytotoxic T lymphocyte assay, Tigges et al., 1996) or by cytokine secretion.

The presence of a cell-mediated immunological response can be determined by proliferation assays (CD4 (+) T cells) or CTL (cytotoxic T lymphocyte) assays. The relative contributions of humoral and cellular responses to the protective or therapeutic effect of an immunogen can be distinguished by separately isolating IgG and T-cells from an immunized syngeneic animal and measuring protective or therapeutic effect in a second subject. As used herein and in the claims, the terms “antibody” or “immunoglobulin” are used interchangeably.

In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

In order to produce monoclonal antibodies, hyperimmunization of an appropriate donor, generally a mouse, with the antigen is undertaken. Isolation of splenic antibody producing cells is then carried out. These cells are fused to a cell characterized by immortality, such as a myeloma cell, to provide a fused cell hybrid (hybridoma) which can be maintained in culture and which secretes the required monoclonal antibody. The cells are then cultured, in bulk, and the monoclonal antibodies harvested from the culture media for use. By definition, monoclonal antibodies are specific to a single epitope. Monoclonal antibodies often have lower affinity constants than polyclonal antibodies raised against similar antigens for this reason.

Monoclonal antibodies may also be produced ex vivo by use of primary cultures of splenic cells or cell lines derived from spleen (Anavi, 1998). In order to produce recombinant antibody (see generally Huston et al., 1991; Johnson et al., 1991; Mernaugh et al., 1995), messenger RNAs from antibody producing B-lymphocytes of animals, or hybridoma are reverse-transcribed to obtain complementary DNAs (cDNAs). Antibody cDNA, which can be full length or partial length, is amplified and cloned into a phage or a plasmid. The cDNA can be a partial length of heavy and light chain cDNA, separated or connected by a linker. The antibody, or antibody fragment, is expressed using a suitable expression system to obtain recombinant antibody. Antibody cDNA can also be obtained by screening pertinent expression libraries.

As used herein and in the claims, the phrase “an immunological portion of an antibody” include a Fab fragment of an antibody, a Fv fragment of an antibody, a heavy chain of an antibody, a light chain of an antibody, an unassociated mixture of a heavy chain and a light chain of an antibody, a heterodimer consisting of a heavy chain and a light chain of an antibody, a catalytic domain of a heavy chain of an antibody, a catalytic domain of a light chain of an antibody, a variable fragment of a light chain of an antibody, a variable fragment of a heavy chain of an antibody, and a single chain variant of an antibody, which is also known as scFv. In addition, the term includes chimeric immunoglobulins which are the expression products of fused genes derived from different species, one of the species can be a human, in which case a chimeric immunoglobulin is said to be humanized. Typically, an immunological portion of an antibody competes with the intact antibody from which it was derived for specific binding to an antigen.

Optionally, an antibody or preferably an immunological portion of an antibody, can be chemically conjugated to, or expressed as, a fusion protein with other proteins. For purposes of this specification and the accompanying claims, all such fused proteins are included in the definition of antibodies or an immunological portion of an antibody.

A method of the present invention includes treatment for a disease or condition caused by a staphylococcus pathogen. In certain aspects the invention encompasses methods of treatment of staphylococcal infection, such as hospital acquired nosocomial infections. In some embodiments, the treatment is administered in the presence of staphylococcal antigens. Furthermore, in some examples, treatment comprises administration of other agents commonly used against bacterial infection, such as one or more antibiotics.

The therapeutic compositions are administered in a manner compatible with the dosage formulation, and in such amount as will be therapeutically effective. The quantity to be administered depends on the subject to be treated. Precise amounts of active ingredient required to be administered depend on the judgment of the practitioner. Suitable regimes for initial administration and boosters are also variable, but are typified by an initial administration followed by subsequent administrations.

The manner of application may be varied widely. Any of the conventional methods for administration of a polypeptide therapeutic are applicable. These are believed to include oral application on a solid physiologically acceptable base or in a physiologically acceptable dispersion, parenterally, by injection and the like. The dosage of the composition will depend on the route of administration and will vary according to the size and health of the subject.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, 12 twelve week intervals, including all ranges there between.

A. Antibodies and Passive Immunization

Certain aspects are directed to methods of preparing an antibody for use in prevention or treatment of staphylococcal infection comprising the steps of immunizing a recipient with a vaccine and isolating antibody from the recipient, or producing a recombinant antibody. An antibody prepared by these methods and used to treat or prevent a staphylococcal infection are a further aspect of the invention. A pharmaceutical composition comprising antibodies that specifically bind IsdA and/or IsdB and a pharmaceutically acceptable carrier is a further aspect of the invention which could be used in the manufacture of a medicament for the treatment or prevention of staphylococcal disease. A method for treatment or prevention of staphylococcal infection comprising a step of administering to a patient an effective amount of the pharmaceutical preparation of the invention is a further aspect of the invention.

Inocula for polyclonal antibody production are typically prepared by dispersing the antigenic composition (e.g., a peptide or antigen or epitope of IsdA or IsdB or a consensus thereof) in a physiologically tolerable diluent such as saline or other adjuvants suitable for human use to form an aqueous composition. An immunostimulatory amount of inoculum is administered to a mammal and the inoculated mammal is then maintained for a time sufficient for the antigenic composition to induce protective antibodies. The antibodies can be isolated to the extent desired by well known techniques such as affinity chromatography (Harlow and Lane, Antibodies: A Laboratory Manual 1988). Antibodies can include antiserum preparations from a variety of commonly used animals e.g., goats, primates, donkeys, swine, horses, guinea pigs, rats or man. The animals are bled and serum recovered.

An antibody produced in accordance with the present invention can include whole antibodies, antibody fragments or subfragments. Antibodies can be whole immunoglobulins of any class (e.g., IgG, IgM, IgA, IgD or IgE), chimeric antibodies, human antibodies, humanized antibodies, or hybrid antibodies with dual specificity to two or more antigens. They may also be fragments (e.g., F(ab′)2, Fab′, Fab, Fv and the like including hybrid fragments). An antibody also includes natural, synthetic or genetically engineered proteins that act like an antibody by binding to specific antigens with a sufficient affinity.

A vaccine of the present invention can be administered to a recipient who then acts as a source of antibodies, produced in response to challenge from the specific vaccine. A subject thus treated would donate plasma from which antibody would be obtained via conventional plasma fractionation methodology. The isolated antibody would be administered to the same or different subject in order to impart resistance against or treat staphylococcal infection. Antibodies of the invention are particularly useful for treatment or prevention of staphylococcal disease in infants, immune compromised individuals or where treatment is required and there is no time for the individual to produce a response to vaccination.

An additional aspect of the invention is a pharmaceutical composition comprising two of more antibodies or monoclonal antibodies (or fragments thereof; preferably human or humanized) reactive against at least two constituents of the immunogenic composition of the invention, which could be used to treat or prevent infection by Gram positive bacteria, preferably staphylococci, more preferably S. aureus or S. epidermidis.

Methods of making monoclonal antibodies are well known in the art and can include the fusion of splenocytes with myeloma cells (Kohler and Milstein, 1975; Harlow Lane, 1988). Alternatively, monoclonal Fv fragments can be obtained by screening a suitable phage display library (Vaughan et al., 1998). Monoclonal antibodies may be humanized or part humanized by known methods.

B. Combination Therapy

The compositions and related methods of the present invention, particularly administration of an antibody that binds IsdA and/or IsdB or a peptide or consensus peptide thereof to a patient/subject, may also be used in combination with the administration of traditional therapies. These include, but are not limited to, the administration of antibiotics such as streptomycin, ciprofloxacin, doxycycline, gentamycin, chloramphenicol, trimethoprim, sulfamethoxazole, ampicillin, tetracycline or various combinations of antibiotics.

In one aspect, it is contemplated that a therapy is used in conjunction with antibacterial treatment. Alternatively, the therapy may precede or follow the other agent treatment by intervals ranging from minutes to weeks. In embodiments where the other agents and/or a proteins or polynucleotides are administered separately, one would generally ensure that a significant period of time did not expire between the time of each delivery, such that the therapeutic composition would still be able to exert an advantageously combined effect on the subject. In such instances, it is contemplated that one may administer both modalities within about 12-24 h of each other and, more preferably, within about 6-12 h of each other. In some situations, it may be desirable to extend the time period for administration significantly, however, where several days (2, 3, 4, 5, 6 or 7) to several weeks (1, 2, 3, 4, 5, 6, 7 or 8) lapse between the respective administrations.

Various combinations of therapy may be employed, for example antibiotic therapy is “A” and an antibody therapy that comprises an antibody that binds IsdA and/or IsdB or a peptide or consensus peptide thereof is “B”:

A/B/A B/A/B B/B/A A/A/B A/B/B B/A/A A/B/B/B
B/A/B/B
B/B/B/A B/B/A/B A/A/B/B A/B/A/B A/B/B/A
B/B/A/A
B/A/B/A B/A/A/B A/A/A/B B/A/A/A A/B/A/A
A/A/B/A

Administration of the antibody compositions of the present invention to a patient/subject will follow general protocols for the administration of such compounds, taking into account the toxicity, if any, of the composition. It is expected that the treatment cycles would be repeated as necessary. It is also contemplated that various standard therapies, such as hydration, may be applied in combination with the described therapy.

C. General Pharmaceutical Compositions

In some embodiments, pharmaceutical compositions are administered to a subject. Different aspects of the present invention involve administering an effective amount of a composition to a subject. In some embodiments of the present invention, an antibody that binds IsdA and/or IsdB or a peptide or consensus peptide thereof may be administered to the patient to protect against or treat infection by one or more bacteria from the Staphylococcus genus. Alternatively, an expression vector encoding one or more such antibodies or polypeptides or peptides may be given to a patient as a preventative treatment. Additionally, such compositions can be administered in combination with an antibiotic. Such compositions will generally be dissolved or dispersed in a pharmaceutically acceptable carrier or aqueous medium.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds of the present invention can be formulated for parenteral administration, e.g., formulated for injection via the intravenous, intramuscular, sub-cutaneous, or even intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions according to the present invention will typically be via any common route. This includes, but is not limited to oral, nasal, or buccal administration. Alternatively, administration may be by orthotopic, intradermal, subcutaneous, intramuscular, intraperitoneal, intranasal, or intravenous injection. In certain embodiments, a vaccine composition may be inhaled (e.g., U.S. Pat. No. 6,651,655, which is specifically incorporated by reference). Such compositions would normally be administered as pharmaceutically acceptable compositions that include physiologically acceptable carriers, buffers or other excipients.

An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the protection desired.

Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

Upon formulation, solutions will be administered in a manner compatible with the dosage formulation and in such amount as is therapeutically or prophylactically effective. The formulations are easily administered in a variety of dosage forms, such as the type of injectable solutions described above.

### V. Examples

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Antibodies that Interfere with Staphylococcus aureus Abscess Formation

Mutations in isdA, isdB and isdC Affect the Pathogenesis of Staphylococcal Infections in Mice.

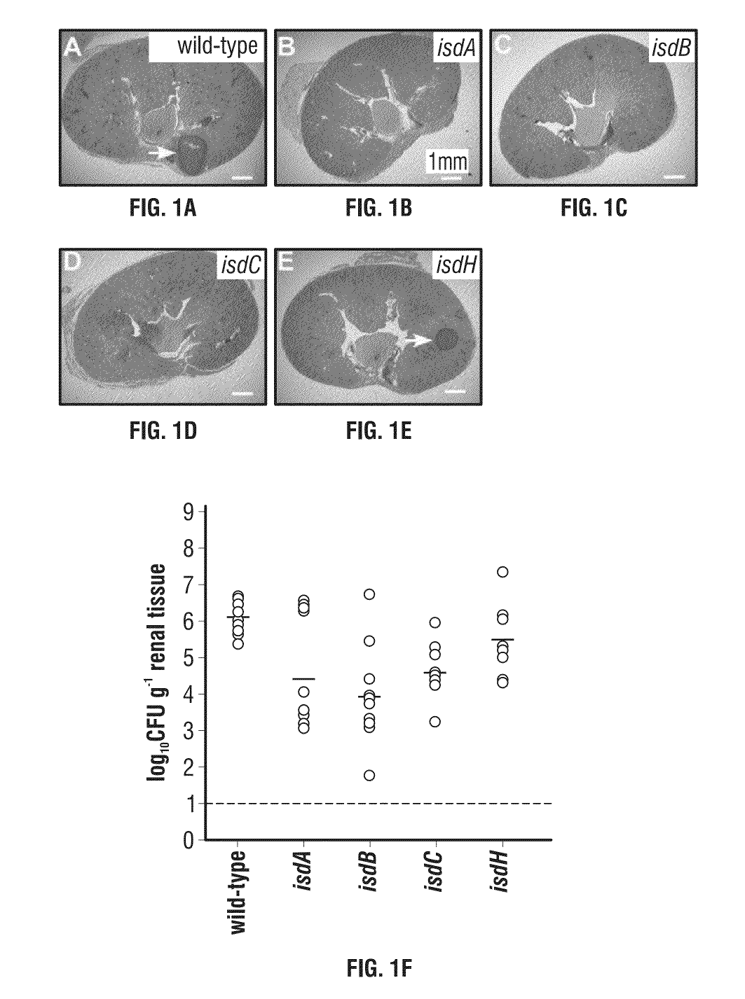

IsdB binds to hemoglobin and scavenges heme, which is transferred first to IsdA and then to IsdC (Liu et al., 2008; Mazmanian et al., 2003; Muryoi et al., 2008). IsdC delivers the tetrapyrrol to IsdEF for transport across the cytoplasmic membrane (Marraffini and Schneewind, 2005; Mazmanian et al., 2002; Zhu et al., 2008). Once within the bacterial cell, IsdG and IsdI cleave heme and liberate iron as nutrient for staphylococcal growth (Skaar et al., 2004; Wu et al., 2004). Previous work asked whether mutations in isdB and isdH, the latter of which encodes a haptoglobin receptor (Dryla et al., 2003; Dryla et al., 2007; Pilpa et al., 2009), affect the pathogenesis of staphylococcal infections in liver or kidney tissues (Torres et al., 2006). Mutations in isdB, but not in isdH, reduced the staphylococcal load four days following intravenous challenge of mice (Torres et al., 2006). These studies left unresolved whether isdB or isdH mutations also impact abscess formation and the ability of mice to survive intravenous high-dose staphylococcal challenge. Bursa aurealis insertions in isdA, isdB and isdC reduced the ability of staphylococci to form abscesses in renal tissue (FIG. 1), a prerequisite for staphylococcal persistence in host tissues (Cheng et al., 2009). In contrast, mutations in isdH did not affect staphylococcal abscess formation (FIG. 1). A mutation in sortase A, which abolishes cell wall anchoring and surface display of all proteins with LPXTG sorting signals (including IsdA, IsdB and IsdH) (Mazmanian et al., 2000; Mazmanian et al., 1999; Mazmanian et al., 2002), caused a severe defect in the pathogenesis of S. aureus lethal disease following intravenous challenge of mice. Reduced virulence defects were also observed for bursa aurealis insertions in isdA, isdB and isdC, but not for isdH (FIG. 1). The inventors conclude that IsdA, IsdB and IsdC mediated heme-iron scavenging from hemoglobin, but not the removal of heme from haptoglobin by IsdH, is required for the pathogenesis of staphylococcal infections in mice.

Purification of Rabbit Antibodies Directed Against IsdA, IsdB or IsdC.

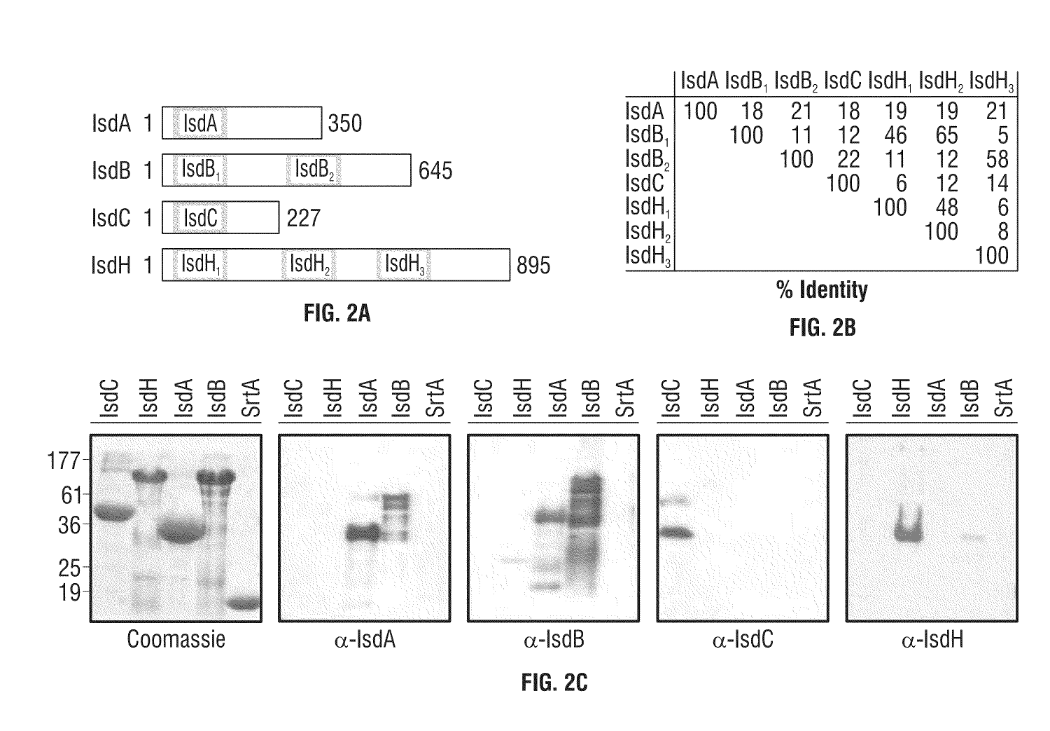

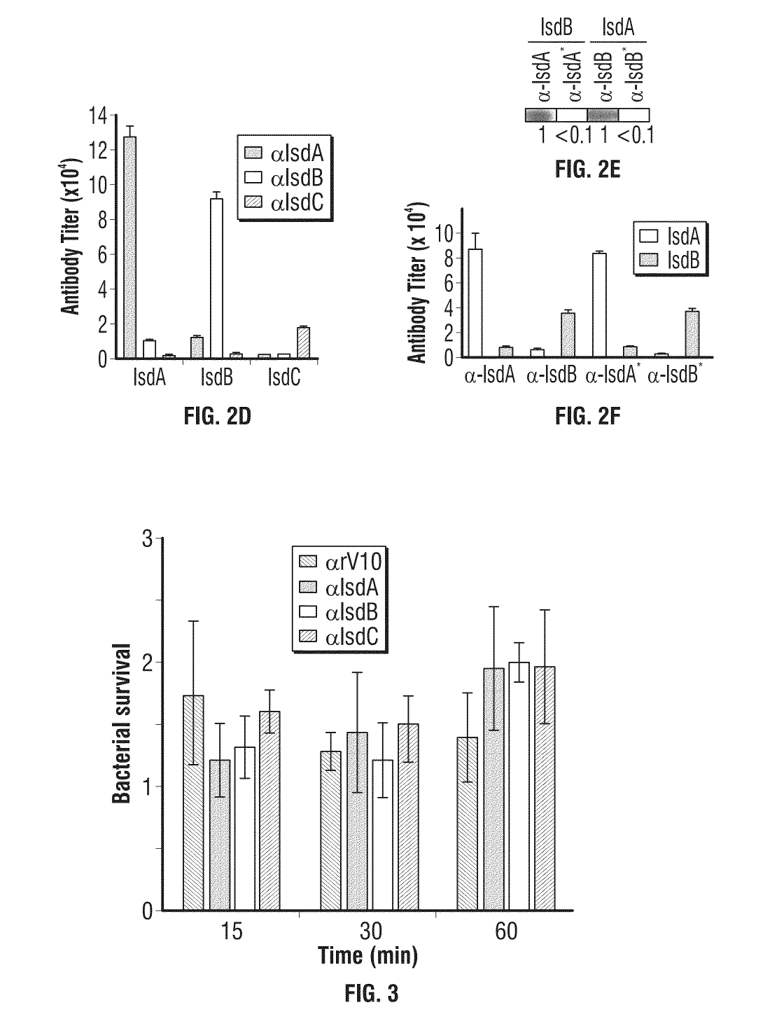

Recombinant IsdA, IsdB, IsdC, and IsdH were expressed in E. coli, purified by affinity chromatography, emulsified in adjuvant and injected into rabbits to generate humoral immune responses (FIG. 2). Antisera were used to blot PVDF membranes with purified proteins, which revealed that IsdA immune serum recognized IsdA and, to a lesser degree, IsdB (FIG. 2). IsdB immune serum recognized IsdB as well as IsdA and IsdH, whereas IsdH antiserum reacted with IsdH and IsdB (FIG. 2). Rabbit antiserum directed against IsdC did not display cross-reactivity to IsdA, IsdB and IsdH (FIG. 2). These observed patterns of cross-reactivity can be explained on the basis of sequence homology and structural similarity between the four NEAT domain containing proteins (Pilpa et al., 2006) (FIG. 2). The N-terminal NEAT domain of IsdB (IsdB1) is most closely related to the second NEAT domain of IsdH (IsdH2—which also binds hemoglobin) (Pilpa et al., 2006) (FIG. 2). The second NEAT domain of IsdB (IsdB2) is most closely related to the NEAT domain of IsdA (Grigg et al., 2007). The NEAT domain of IsdC, a sortase B anchored product that functions as the central conduit for heme-iron transport in staphylococci (Mazmanian et al., 2002) appears unique in sequence and structure (Mazmanian et al., 2003; Sharp et al., 2007; Villaral et al., 2008). To evaluate the functional properties of rabbit humoral immune responses, the inventors purified IsdA, IsdB and IsdC specific antibodies by binding to affinity matrices comprised of purified IsdA, IsdB and IsdC. Crossreactive IsdA and IsdB antibodies were removed by chromatography on the reciprocal affinity matrix. The specificity of eluted IsdA and IsdB antibodies was verified by immunoblotting against purified recombinant protein (FIG. 2).

IsdA, IsdB and IsdC Antibodies Cannot Promote Opsonophagocytosis of Staphylococci in Mouse Blood.

Gram-positive bacteria, such as S. aureus, cannot be killed by complement lysis alone, and host clearance of these pathogens requires opsonophagocytosis and lysis by immune cells (Lancefield, 1962). Previous studies reported that antibodies against IsdA and IsdB promote opsonophagocytic killing of staphylococci by isolated human blood polymorphonuclear leukocytes (PMNs) (Stranger-Jones et al., 2006). In this assay, also developed for the characterization of antibodies that were generated in response to immunization with capsular polysaccharide-conjugates (Fattom et al., 2004), staphylococci are incubated with a ten-fold excess of isolated human PMNs as well as rabbit complement and antiserum pre-adsorbed to staphylococci (Stranger-Jones et al., 2006). However, wild-type staphylococci display protein A to capture immunoglobulins via their Fc portion on the bacterial surface (Jensen, 1958; Sjoquist et al., 1972). Previous experiments therefore examined the opsonophagocytic properties of antibodies against surface proteins with a protein A mutant strain (Stranger-Jones et al., 2006). Here opsonophagocytosis was tested under physiological conditions, as occurs when wild-type staphylococci enter the bloodstream of their host (Lancefield, 1928). Fresh blood was isolated from naive mice (lacking staphylococcal antibodies) via cardiac puncture and coagulation blocked with lepirudin. Survival of wild-type S. aureus Newman rotating in blood was monitored 15, 30, and 60 min after the addition of 1×10⁵ CFU (FIG. 3). When mock (PBS) treated or incubated in the presence of an irrelevant antibody, 1 μg/ml purified rabbit anti-V10 [IgG directed against LcrV, a Yersinia pestis protective antigen that is not expressed by staphylococci (Overheim et al., 2005)], S. aureus Newman was not killed in mouse blood. This result corroborates the recent observation that, following intravenous challenge, staphylococci cannot be cleared in the blood of naive animals (Cheng et al., 2009; Thammavonga et al., 2009). Addition of 1 μg purified rabbit antibodies directed against IsdA, IsdB, or IsdC per ml blood did not enable immune cells to phagocytose and kill S. aureus Newman (FIG. 3, Table 1). Of note, antibody reagents and preparation of staphylococci for in vitro opsonophagocytosis in mouse blood were similar to experimental conditions demonstrating protection against staphylococcal abscess formation and for lethal challenge (vide infra). The inventors examined whether passive transfer of purified antibodies into the peritoneal cavity of mice (5 mg kg⁻¹ weight administered 24 hours prior to challenge) caused a significant reduction of S. aureus Newman in the blood stream 60 minutes following intravenous challenge with 1×10⁷ CFU. IsdA antibodies also did not promote opsonophagocytic clearance of staphylococci in the bloodstream of BALB/c mice (data not shown). The inventors conclude that rabbit antibodies against IsdA, IsdB and IsdC cannot promote early opsonophagocytic clearance of wild-type staphylococci in mouse blood and that the protective value of these antibodies must be derived from neutralizing the important virulence attributes of proteins within the iron-regulated surface determinant system (Isd).

TABLE 1
Survival of S. aureus Newman in mouse blood is not affected by
antibodies directed against iron-regulated surface determinants

| | Survival^{b} | P-value^{c} | Survival^{b} | P-value^{c} | Survival^{b} | P-value^{c} |
|---|---|---|---|---|---|---|
| Antibody^{a} | 15 min | | 30 min | | 60 min | |
| ^{d}V₁₀ | 1.76 ± 0.58 | — | 1.28 ± 0.15 | — | 1.39 ± 0.35 | — |
| IsdA | 1.22 ± 0.28 | 0.3474 | 1.43 ± 0.50 | 0.7500 | 1.94 ± 0.51 | 0.2911 |
| IsdB | 1.32 ± 0.25 | 0.4226 | 1.21 ± 0.30 | 0.6977 | 2.00 ± 0.15 | 0.3113 |
| IsdC | 1.59 ± 0.17 | 0.7981 | 1.46 ± 0.27 | 0.3206 | 1.97 ± 0.46 | 0.2914 |

^{a}Affinity purified rabbit antibodies (1 μg/ml) was added to 1 ml of mouse blood and infected with 1 × 10⁵ CFU S. aureus strain Newman.
^{b}Bacterial survival reported as [(staphylococcal CFU at observation period/staphylococcal CFU input)*100] in presence of antibody/staphylococcal CFU/staphylococcal CFU input)*100] normalized to PBS alone.
^{c}P-values were determined using the Student's t-test.
^{d}V10 antibodies are directed against LcrV, a protective antigen of Yersinia pestis that is not expressed by S. aureus.

IsdA and IsdB Antibodies Protect Against Staphylococcal Abscess Formation and Lethal Challenge.

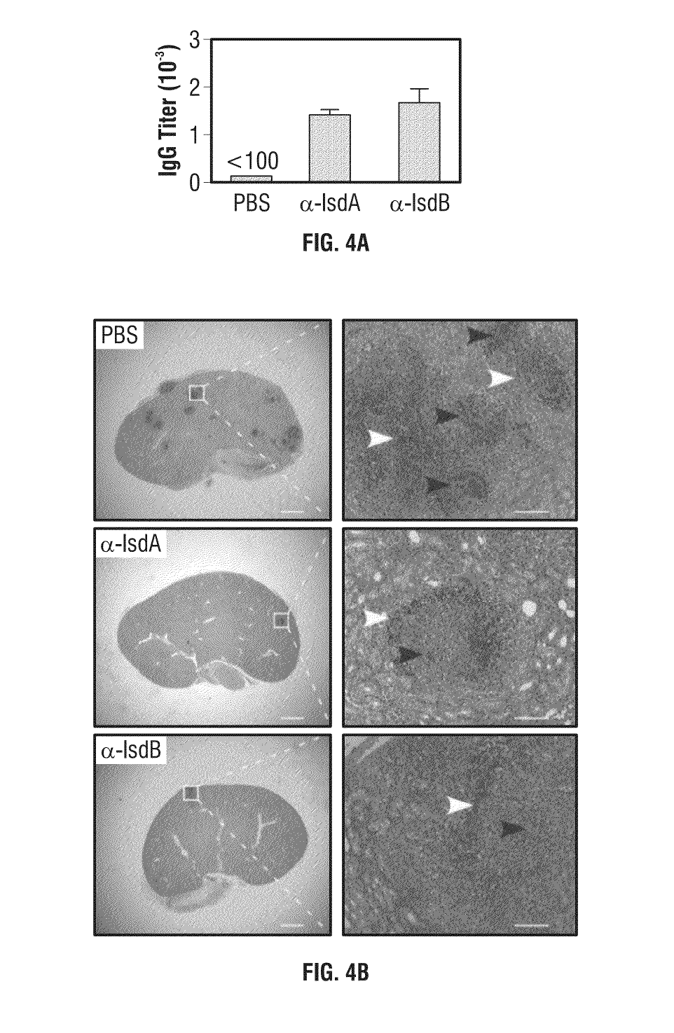

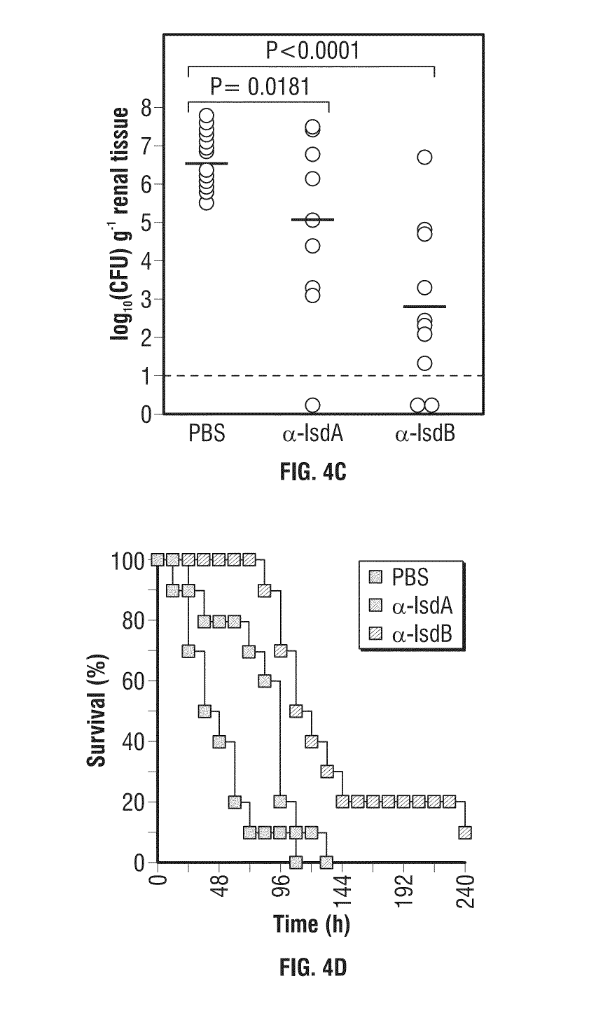

Affinity purified antibodies or mock control (PBS) were injected into the peritoneal cavity of naive BALB/c mice (5 mg IgG kg⁻¹ body weight). Twenty-four hours following passive immunization, serum antibody concentration was examined by ELISA. Dilution endpoints revealed a significant amount of serum IgG for IsdA [1,400 (±126 SEM)] or IsdB [1,640 (±320 SEM)](Table 2). Passively immunized animals were challenged by intravenous injection with 1×10⁷ CFU S. aureus Newman. Animals were killed four days following challenge and kidneys removed. To enumerate staphylococcal load in renal tissue, homogenate from one of the two kidneys was spread on agar media and incubated for colony formation. Compared to a PBS control, passive immunization with IsdA or IsdB antibodies caused to a significant reduction in staphylococcal load (FIG. 4 and Table 2). To quantify abscess formation, randomly chosen kidneys were fixed in formalin, embedded; thin sectioned, and stained with hematoxylin-eosin. For each kidney, four sagittal sections at 200 μM intervals were viewed by microscopy to determine the number of abscess lesions for each organ. In mock immunized mice, S. aureus Newman caused an average of 4.64 (±1.17) abscesses per kidney (Table 2). Animals immunized with IsdA or IsdB-specific IgG harbored an average of 1.0 (±0.5) and 0.86 (±0.46) abscesses per organ, respectively (Table 2). Compared to mock immunized control animals, abscess lesions of IsdA or IsdB immunized animals appeared smaller, with massive PMN infiltrates, fewer necrotic immune cells and, located at the center, staphylococcal abscess communities that was diminished in size (FIG. 4).

TABLE 2
Antibodies against IsdA and IsdB protect against staphylococcal abscess formation

| | | Staphylococcal load in kidneys^{c} | | | Abscess formation | | |
|---|---|---|---|---|---|---|---|
| Antibodies^{a} | IgG titer^{b} | Log₁₀ CFU^{d} | Reduction^{c} | P value^{f} | Abscesses^{g} | Reduction^{h} | P value^{j} |
| Mock^{j} | — | 6.59 ± 0.15 | — | — | 4.64 ± 1.17 | — | — |
| IsdA | 1,400 ± 126 | 5.11 ± 0.79 | 1.48 | 0.0181 | 1.0 ± 0.5 | 3.64 | 0.0268 |
| IsdB | 1,640 ± 320 | 2.83 ± 0.68 | 3.76 | 0.0001 | 0.86 ± 0.46 | 3.78 | 0.0395 |
| IsdB_{N} | 875 ± 97 | 5.54 ± 0.69 | 1.05 | 0.0551 | 1.4 ± 0.54 | 3.24 | 0.0382 |
| isdB_{C} | 910 ± 114 | 3.51 ± 0.99 | 3.08 | 0.0001 | 1.2 ± 0.68 | 3.44 | 0.0327 |

^{a}Affinity purified antibodies were injected at IgG concentration of 5 mg kg⁻¹ animal weight in 100 μl PBS into the peritoneal cavity of naïve 6 week old female BALB/c mice.
^{b}Antibody titers were analyzed by ELISA with purified recombinant antigen (1 μg) by dilution of serum samples derived via cardiac puncture 24 hours following passive immunization.
^{c}Four days following intravenous challenge with 1 × 10⁷ CFU S. aureus Newman, animals were killed, kidneys excised and tissue homogenate from one randomly chosen kidney spread on agar plates for colony formation and enumeration. Data represent the average and standard error of the means of the log₁₀ CFU from 10 kidneys. Twenty kidneys were analyzed for mock immunized animals injected with 100 μl PBS.
^{e}Reduction in log₁₀ CFU compared to the mock control.
^{f}Statistical significance was analyzed with the two-tailed Student's t-test and P values recorded.
^{g}Average number (and standard error of the means) of abscesses formed in the kidneys of infected mice was enumerated by histophathology of thin-sectioned hematoxylin-eosin stained tissue samples.
^{h}Reduction in the number of abscesses compared to the mock control.
^{j}Statistical significance was analyzed with the two-tailed Student's t-test and P values recorded.

To study whether passive transfer of purified IsdA and IsdB-specific antibodies confers protection against lethal disease, cohorts of mice were injected with 5×10⁸ CFU S. aureus Newman and monitored for survival over the next 240 hours. Most of the mock immunized animals (80%) survived staphylococcal challenge for up to 48 hours and the remaining animals died within 100 hours (FIG. 4). Animals immunized with IsdA-specific IgG survived for a longer time interval: 80% of animals died 96 hours following challenge, whereas the death of the remaining animals occurred by 124 hours. Animals immunized with IsdB-specific IgG displayed the most significant protection: 10% of mice in this cohort did not suffer from lethal disease at the end of the 240 hour observation period. Most of the IsdB immunized animals (80%) survived for at least 144 hours (FIG. 4).

IsdB Specific Antibodies Block Hemoglobin Binding and Heme Transfer.

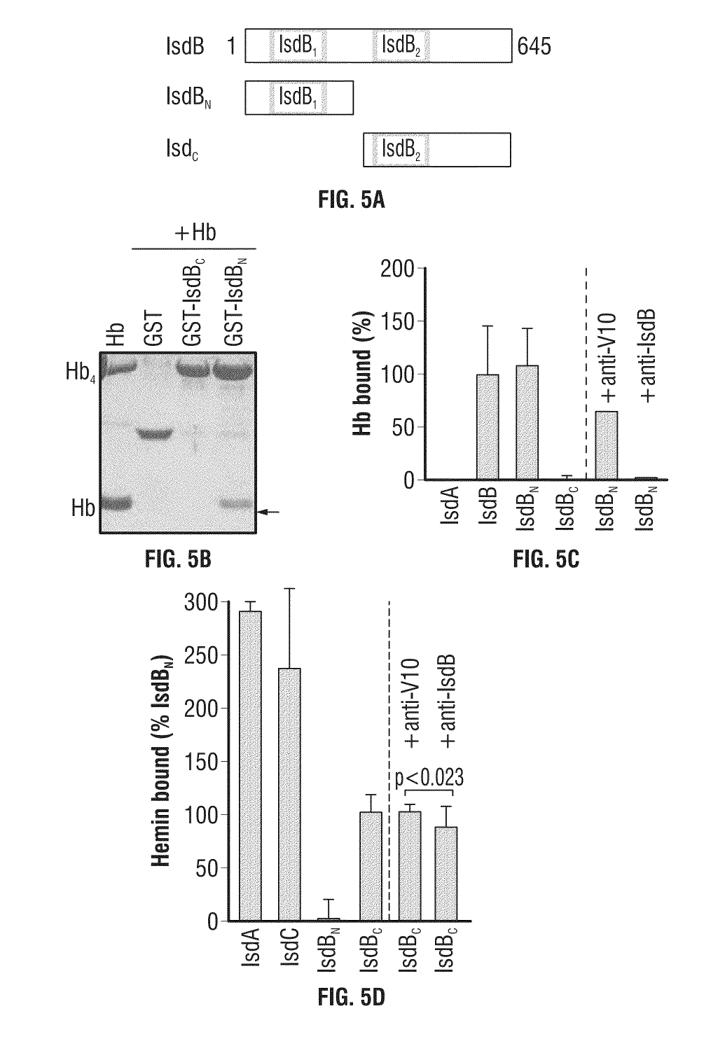

To address the molecular mechanism of antibody protection, recombinant IsdB was purified and its ability to bind hemoglobin and heme was measured by surface plasmon resonance (SPR) or binding to TMBZ (Stugard et al., 1989), respectively. As previously reported, IsdB binds both hemoglobin and heme, which is attributed to the two NEAT domains, IsdB1 and IsdB2. To distinguish the effect of antibodies on the two biochemical activities of IsdB, recombinant DNA fragments were engineered to cut the molecule into halves, IsdB_{N} (including IsdB1) and IsdB_{C} (with IsdB2) (FIG. 5). As reported earlier, purified IsdB_{N} bound to hemoglobin, but not heme, whereas IsdB_{C} used heme as a ligand, but not hemoglobin (FIG. 5). These findings are in agreement with the general property of IsdB for removing heme from hemoglobin and transferring the iron-tetrapyrrol to IsdA. Affinity purified rabbit antibodies directed against IsdB (full length) blocked the ability of IsdB_{N} to bind hemoglobin (FIG. 5). The same antibody sample significantly reduced heme binding of IsdB_{C} but could not altogether prevent the polypeptide's association with iron-tetrapyrrol (FIG. 5). From these data the inventors conclude that antibodies against IsdB interfere with both biochemical attributes of the surface protein, i.e., capturing hemoglobin and removing heme, albeit that the latter reaction is only inhibited in part.

The Protective Values of IsdB_{N} and IsdB_{C}-Specific Antibodies.

Rabbits were immunized with purified IsdB_{N} or IsdB_{C} and antigen specific antibodies were purified by affinity chromatography. Antibodies against IsdB_{N} or IsdB_{C} as well as a PBS control were injected into the peritoneal cavity of naive BALB/c mice (5 mg IgG kg⁻¹ body weight). Twenty-four hours following passive immunization, serum antibody concentration was examined by ELISA as dilution endpoints for IsdB_{N [}900 (±126 SEM)] and IsdB_{C [}950 (±320 SEM)] (FIG. 6). Additional animals in the same cohorts were challenged by retro-orbital injection with 1×10⁷ CFU S. aureus Newman. Animals were killed four days following challenge and kidneys removed. Compared to the PBS control, IsdB_{N} and IsdB_{C} specific antibodies led to a significant reduction in bacterial load four days after challenge (FIG. 6). Immunization with IsdB_{N} and IsdB_{C} antibodies also reduced the number of abscess lesions quantified during histopathology of kidney tissue (Table 2). When tested for the ability to protect against staphylococcal lethal challenge, antibodies directed against IsdB_{N} and IsdB_{C} alone both extended the survival of passively immunized mice. Of note, the combined administration of antibodies against IsdB_{N} and IsdB_{C} generated increased survival as compared to mice that received each of the two antibodies alone (FIG. 6). In summary, IsdB specific antibodies directed against the hemoglobin receptor domain or the heme transfer domain generate moderate levels of protection against staphylococcal abscess formation or lethal challenge. Disease protection is increased by combining antibodies specific for each of the two domains, in agreement with the hypothesis that IsdB immunization interferes with the heme-iron scavenging of S. aureus in host tissues.

Protective Value of IsdA- and IsdB-Specific MAbs.

Overnight cultures of S. aureus were refreshed 1:100 in TSB and grown for two hours to an OD₆₆₀ of 0.4. Staphylococci were sedimented, washed and suspended in PBS to the desired bacterial concentration. Inocula were quantified by spreading sample aliquots on TSA and enumerating the colonies that formed upon incubation. Purified MAbs were injected into the peritoneal cavity of 6 week old female BALB/c mice (Charles River, cohorts of ten animals) at a concentration of 5 mg kg⁻¹ (typically 100 μg per animal of 20 g body weight). Four hours later, staphylococci were used to infect anesthetized mice by retro-orbital injection (1×10⁷ CFU of S. aureus Newman). BALB/c mice were anesthetized via intraperitoneal injection with 100 mg·ml-1 ketamine and 20 mg·ml-1 xylazine per kilogram of body Weight. 1×10⁷ CFU S. aureus Newman were injected into mice via retro-orbital injection. FiveFour days post-infection mice were killed by CO2 inhalation, kidneys removed, and fixed in 10% formalin for histopathology or homogenized and serial dilutions spread on tryptic soy agar (TSA) to determine colony forming units (CFU). Fixed tissues were embedded in paraffin, thin-sectioned, stained with hematoxylin-eosin, and inspected by light microscopy to enumerate abscess lesions.

Protection was analyzed as the ability of MAbs to reduce the bacterial load in kidney tissue (recorded as log₁₀CFU reduction) compared to isotype matched control antibodies unable to recognize staphylococcal antigens (Table 3). As these data abide by a normal distribution, the unpaired two-tailed student's t-test was used to analyze statistical significance (P≦0.05 was judged as significant, Table 3). Animal experiments were performed in accordance with the institutional guidelines following experimental protocol review and approval by the Institutional Biosafety Committee (IBC) and the Institutional Animal Care and Use Committee (IACUC) at the University of Chicago.

Two antibodies raised against IsdB, MAb 3D8 and 4H7, caused a significant reduction (3.21 and 2.01 log₁₀ CFU per organ, respectively) in bacterial load four days after S. aureus Newman challenge, whereas the other antibody derived from IsdB immunization, MAb 2A9, did not generate protection (Table 3). Six different MAb antibodies raised via IsdA immunization were protective and caused a significant reduction in staphylococcal load: 4B9 (4.04 log₁₀ CFU per organ), 7E9 (2.81 log₁₀ CFU per organ), 1B8 (2.34 log₁₀ CFU per organ), 5H8 (2.15 log₁₀ CFU per organ), 7D4 (2.04 log₁₀ CFU per organ) and 6A11 (1.95 log₁₀CFU per organ). In contrast, six MAbs failed to raise significant protection: 6H4, 5F6, 3H11, 6A4, 6A11, and 3E8 (Table 3).

Ability of IsdA and IsdB MAbs to Induce Killing of Staphylococci in Blood.

Gram-positive bacteria, such as S. aureus, cannot be killed by complement lysis alone. Host clearance of this pathogen typically involves opsonophagocytic killing by immune cells (Lancefield, 1928). Previous work reported that IsdA- or IsdB-specific antibodies promote opsonophagocytic killing of staphylococci by isolated human PMNs and baby rabbit complement, however polyclonal IsdA and IsdB antibodies that exert protection against staphylococcal infection did not induce clearance of the bacteria in lepirudin-treated mouse blood (Kim et al., 2010). The inventors tested IsdA- and IsdB-specific MAbs for staphylococcal killing in mouse blood. Fresh blood was isolated from naïve BALB/c mice via cardiac puncture or from human volunteers in accordance with the institutional guidelines following experimental review, approval, and guidance by The University of Chicago Institutional Review Board (IRB). Blood coagulation was blocked with 10 μg ml⁻¹ lepirudin. The absence of staphylococcal antibodies in blood of naïve mice was determined by incubating serum with the staphylococcal antigen-matrix, a collection of 27 recombinant purified proteins that are known to reside in the bacterial envelope or to be secreted into the culture medium (Kim et al., 2010). The survival of wild-type S. aureus Newman rotating in lepirudin-treated blood was monitored 30 min (mouse blood) or 120 minutes (human blood) after the addition of 5×10⁵ CFU (5×10⁶ CFU for human blood experiments) in the presence of 2 μg ml-1 individual IsdA or IsdB MAbs or matched isotype controls. For survival in human blood, bacteria were grown in chelex treated RPMI plus 0.2 M 2′-2-dipyridyl. Blood was collected and incubated on ice with 1% saponin/PBS. Serial dilutions were plated on TSA for CFU determination. Antibody reagents and preparation of staphylococci for in vitro opsonophagocytosis in mouse blood were similar to experimental conditions testing protection against staphylococcal abscess formation (vide infra), with blood collected 60 min. following infection.

Mock treatment with IgG from naïve mice, 5 μg ml⁻¹, did not affect staphylococcal survival in blood. Addition of 5 μg ml⁻¹ purified IsdA- or IsdB-specific MAbs per 1 ml blood had variable effects on the killing of S. aureus Newman (Table 3). IsdB-derived MAb 3D8, but not MAbs 4H7 and 2A9, caused a significant degree of staphylococcal killing in mouse blood. Further, IsdA-derived MAbs 4B9, 1B8, 5H8, 6A4 and 6H4 triggered significant bacterial killing, whereas MAbs 7E9, 7D4, 6A11, 3H11, 5F6 and 3E8 did not. Two MAbs with very good value for disease protection in vivo, IsdB-derived 3D8 and IsdA-derived 4B9, also caused staphylococcal killing in mouse blood. Nevertheless, a positive correlation between different MAbs that trigger staphylococcal killing in mouse blood and the ability to protect from disease was not observed, as two antibodies with the highest killing activities, 6H4 and 6A4, afforded no significant protection from staphylococcal disease (Table 3). Further, some antibodies that did raise disease protection failed to trigger staphylococcal killing (4H7, 7E9, 7D4, and 6A11). Of note, antibody reagents and preparation of staphylococci for in vitro opsonophagocytosis in mouse blood were similar to experimental conditions testing protection against staphylococcal abscess formation.

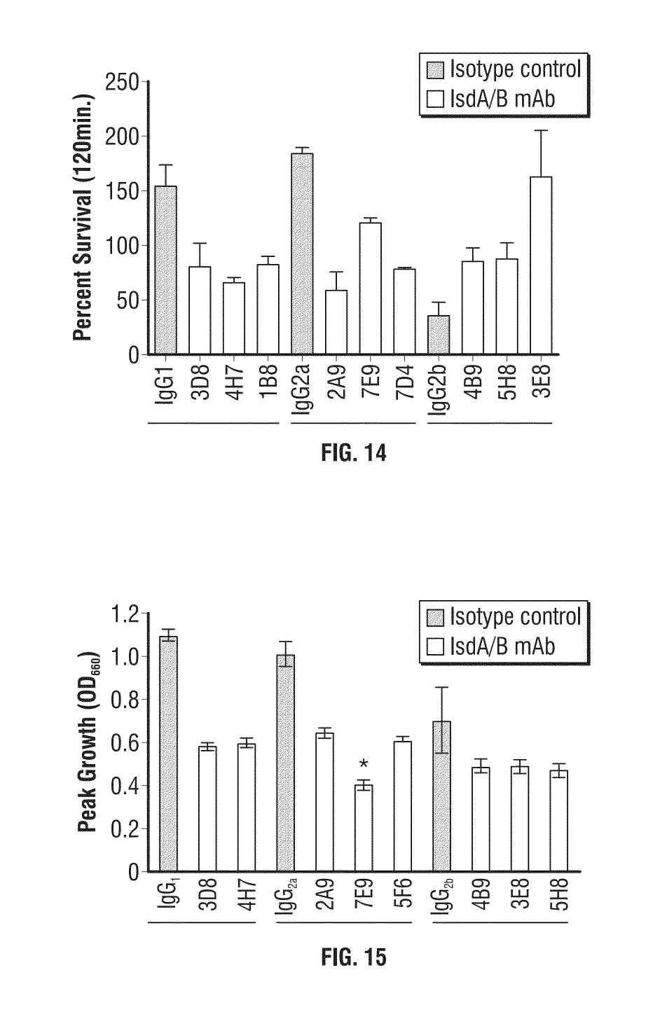

The survival of staphylococci was also examined in the presence of IsdA or IsdB specific MAbs in human blood when bacteria were first grown under iron limiting conditions to induce IsdA and IsdB antigen expression (FIG. 14). Although statistical significance was not calculated for both IgG₁ and IgG₂ₐ type Isd MAbs examined, bacterial survival was reduced compared to isotype controls. However, as the isotype control IgG_{2b} resulted in the greatest reduction in survival, and the decrease in survival does not directly correlate with protection in the mouse, immunoglobulin mediated killing in this assay may not be directly related to specific recognition of IsdA and/or IsdB bacterial surface antigens.

Ability of MAbs to Block IsdA or IsdB Binding to Heme.

As one of the major pathways for iron acquisition of S. aureus during infection (Skaar et al., 2004), the Isd system has an important role in the pathogenesis of this organism (Cheng et al., 2009; Kim et al., 2010). Several of the members of the Isd pathway (IsdA, IsdB, IsdC and IsdH) contain NEAT domains (NEAr iron transporters) that bind heme (Grigg et al., 2007; Pilpa et al., 2006) and are involved in the passage of heme across the bacterial cell envelope and into the cell (Mazmanian et al., 2003). To address the ability of MAbs to disrupt heme-binding, GST-tagged IsdA and IsdB_{C} (Kim et al., 2010) were purified and incubated with individual IsdA or IsdB MAbs. Following incubation with hemin-chloride, absorbance spectroscopy was used to monitor heme binding (Skaar et al., 2004). Specifically, 3 μM protein was incubated with 3 μM of individual MAbs, incubated for 30 minutes at 25° C. and absorbance measured from 300-600 nm. Hemin chloride, 20 μM for IsdA or 30 μM IsdB_{C} was added and reactions incubated at 25° C. for an additional 10 minutes, followed by measurement of peak absorbance from 300-600 nm. Three antibodies resulted in a change in the absorbance profile as compared to protein and hemin alone (Table 3). The Soret peak at 405 nm is indicative of heme binding and MAbs 7E9, 3D8, and 4H7 all shifted the heme absorbance peak to the left for both IsdA and IsdB_{C}, suggesting that these MAbs blocked the access of IsdA and IsdB to heme.

TABLE 3
Biochemical attributes and biological values of MAbs raised against IsdA and IsdB

| | | | | | | | | | Heme- |
|---|---|---|---|---|---|---|---|---|---|
| | | | | | Abscess formation | | Survival in mouse blood | | transport |
| | | | Affinity^{d} | | Reduction^{e} | Significance^{f} | Survival | Significance^{h} | Δheme |
| MAb^{a} | Antigen^{b} | Class^{c} | IsdB | IsdA | log₁₀ CFU | P-value | (%)^{g} | P-value | binding^{j} |
| 3D8 | IsdB | IgG1 | 7.46 | 7.19 | 3.21 | 0.01 | 63(±4) | 0.002 | 34/29.4 |
| 4H7 | IsdB | IgG1 | 3.09 | 6.86 | 2.01 | 0.05 | 81 ±7) | 0.064 | 34.4/29 |
| 2A9 | IsdB | IgG2a | 2.74 | 0.03 | 1.07 | 0.24 | 141(±13) | NSR | 2.9 |
| 4B9 | IsdA | IgG2b | 1.14 | 0.13 | 4.04 | 0.001 | 50(±5) | 0.001 | 0 |
| 7E9 | IsdA | IgG2a | 5.36 | 17.59 | 2.81 | 0.01 | 88(±38) | 0.759 | 35/29 |
| 1B8 | IsdA | IgG1 | 1.93 | 2.63 | 2.34 | 0.04 | 35(±14) | 0.010 | 0.5 |
| 5H8 | IsdA | IgG2b | 0 | 4.39 | 2.15 | 0.02 | 52(±10) | 0.009 | 0.5 |
| 7D4 | IsdA | IgG2a | 0 | 18.67 | 2.04 | 0.004 | 135(±23) | NSR | −0.6 |
| 6A11 | IsdA | IgG2b | 10.04 | 11.09 | 1.95 | 0.05 | 108(±27) | 0.771 | 0 |
| 6A4 | IsdA | IgG2b | 6.0E−28 | 1.74 | 1.94 | 0.11 | 50(±7) | 0.003 | 0 |
| 3H11 | IsdA | IgG2b | 0 | 13 | 1.82 | 0.07 | 106(±5) | 0.345 | 0.5 |
| 5F6 | IsdA | IgG2a | 14.14 | 17.71 | 1.53 | 0.13 | 100(±14) | 0.980 | −0.6 |
| 6H4 | IsdA | IgG1 | 0.005 | 3.9 | 1.40 | 0.24 | 38(±3) | <0.001 | 1 |
| 3E8 | IsdA | IgG2b | 0 | 5.07 | 0.55 | 0.55 | 94(±9) | 0.530 | 0 |

^{a}Mouse monoclonal antibodies were purified from isolated hybridoma clones
^{b}Antigen used to elicit mouse monoclonal antibodies
^{c}Immunoglobulin call and subclass of MAbs
^{d}Affinity was determined by ELISA as the association constant (Kₐ) in nM for either IsdA or IsdB. ND, not determined.
^{e}Disease protection in BALB/c mice (cohorts of 10 animals) was analyzed by intraperitoneal injection of purified MAb (5 mg kg⁻¹) 4 hours prior to retro-orbital challenge with 1 × 10⁷ CFU S. aureus Newman. Animals were killed 4 days after challenge and staphylococcal load in renal tissue homogenate determined by dilution and colony formation. Disease protection was recorded as the log₁₀ CFU reduction in staphylococcal load as compared to an isotype matched control MAb that did not bind to staphylococci.
^{f}Statistical significance of disease protection data was calculated with the unpaired two-tailed student's t-test and P-values recorded.
^{g}Opsonophagocytosis mediated killing of 5 × 10⁵ CFU S. aureus Newman in 30 minutes by 1 ml lepirudin-treated (10 μg ml⁻¹) fresh mouse blood with 5 μg ml⁻¹ isotype matched control MAb or MAbs derived from IsdA-/ IsdB-immunization of mice. S. aureus survival in the presence of control MAb was set as 100% and relative survival in the presence of IsdA-/IsdB-derived MAb was calculated. Standard error of the means recorded in parenthesis.
^{h}Statistical significance of opsonophagocytic killing was analyzed with the unpaired two-tailed student's t-test and P-values recorded. NSR, no significant reduction.
ⁱInhibition of IsdB-binding to hemoglobin as assayed.
^{j}Values represent the percent change in Δ peak absorbance at 405 nm and 368 nm for absorbance of IsdA · heme complexes formed in the presence of MAb as compared to the absence of antibody. Two values indicate the percent change of adsorbance for IsdA · heme and IsdB_{C} · heme complexes in the presence of the same MAb.

IsdA/B MAb Interferes with S. aureus Growth when Hemoglobin is the Sole Iron Source

The Isd heme iron acquisition system represents an important pathway for staphylococcal survival in iron limiting environments such as those present within the host as discussed above. Since IsdB has been identified as the Isd hemoglobin receptor, important for binding to and extracting the heme from the hemoprotein, and 3 of the 11 antibodies were able to interfere with heme binding to purified IsdA and IsdB, tests were completed to assess the ability of some of the antibodies to interfere with the cells ability to acquire heme iron from human hemoglobin. To this end, growth of iron starved staphylococcal cells was monitored over a period of 16 hours with freshly purified human hemoglobin as the major source of iron. Cells were grown in the presence or absence of hemoglobin, with growth only resulting when hemoglobin was present. As a control for possible iron or heme contamination in antibody preparations, cells were also grown in the presence of 20 μg of individual IsdA/B MAbs or isotype matched controls, in the presence or absence of hemoglobin. Some growth occurred in the presence of antibodies alone, indicating the presence of background iron sources, however it was much reduced compared to when hemoglobin was added.

Peak growth for each sample, measured as A₆₆₀, was determined and averages plotted (FIG. 15). All antibodies tested resulted in a reduction in peak absorbance, with additional variation seen in the kinetics of growth for each. However, following statistical analysis using the Kruskal Wallis analysis of variance, only IsdA MAb 7E9 resulted in a significant reduction in peak growth.

IsdA and IsdB MAb Affinity Mapping with IsdA Deletion Variants

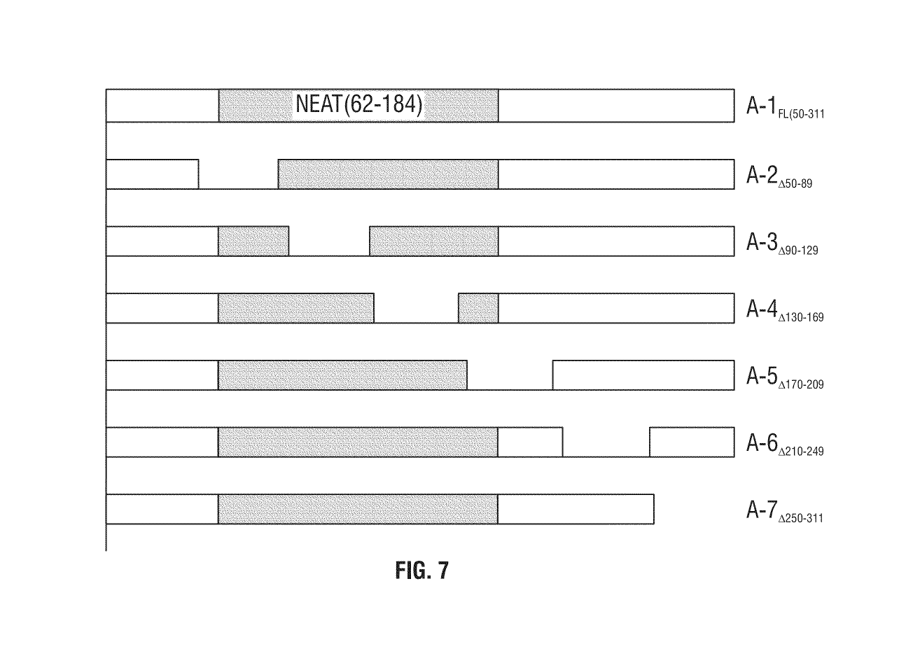

Poly-histidine tagged IsdA mutant protein variants, IsdA-1_{FL50-311}, IsdA-2_{Δ50-89}, IsdA-3_{Δ90-129}, IsdA-4_{Δ130-169}, IsdA-5_{Δ170-209}, IsdA-6_{Δ210-249}, IsdA-7_{Δ250-311} (FIG. 7A) were purified by affinity chromatography, analyzed by SDS-PAGE and visualized via Coomassie stain. Nunc MaxiSorp 96-well plates were coated with IsdA variants at a concentration of 1 μg ml⁻¹ in 0.1M sodium bicarbonate. Plates were blocked with 3% BSA in PBS, followed by incubation with variable concentrations of MAbs in PBS-Tween. The affinity of MAbs to bind each IsdA variant was measured as the concentration of bound/free antibody using secondary antibody-HRP conjugates and chemiluminescence detection. Using this data, the association constant of antibody for antigen was calculated (Table 4).

Isd MAbs, including those generated against IsdB antigen, bound full length IsdA protein to varying degrees. Deletion of residues 50-89 and 130-209, both containing regions of the NEAT domain, resulted in a large reduction in Kₐ for all MAbs, indicating these regions are necessary for all 14 antibodies to bind IsdA antigen. Residues 210-249, which does not comprise the NEAT domain, resulted in a loss of binding (a reduction in K_{A}) for all MAbs with the exception of 7E9. Loss of binding following deletion of more than one region that are not consecutive suggests that the epitopes are conformational. Also of note is that deletion of amino acids 90-129 and the most C-terminal amino acids, 250-311, resulted in increased binding for many of the MAbs. Specifically, deletion of residues 90-129, which encompasses a portion of the NEAT domain, resulted in increased binding of 3D8, 4H7, 2A9, 4B9, 7E9, 5H8, 6A11, 6A4, 5F6, and 3E8 while deletion of the C-terminus, amino acids 250-311, resulted in increased binding of 2A9, 7E9, 1B8, 5H8, 6A4, and 3E8.

Discussion

The foregoing studies show that antibodies directed against IsdA, which do not cross-react with IsdB (5H8 and 7D4), are able to protect against S. aureus challenge. These antibodies do (5H8) or do not (7D4) affect staphylococcal survival in fresh blood, suggesting that they may also have the potential to block heme-iron binding and or heme-transport across the staphylococcal envelope in vivo (Mazmanian et al., 2003). Several other IsdA-derived MAbs crossreact and bind with similar affinity to both IsdA and IsdB (4B9, 7E9, 1B8, 6A11 and 5F6). Two of these antibodies reduce the survival of staphylococci in mouse blood and may at least in part exert their protective attribute in this manner. The third (7E9) has no effect on the ability of staphylococci to survive in blood. IgG1 and IgG2a IsdA and B MAbs also reduced the growth of pre iron-starved staphylococcal survival in human blood compared to their isotype control. As indicated by its ability to block heme binding for both IsdA and IsdBC and statistically reduce staphylococcal growth when hemoglobin is the sole iron source, MAb 7E9 interferes with the heme-iron transport pathway of staphylococci. In summary, our data demonstrate that antibodies derived via IsdA immunization provide disease protection and that these antibodies are either specific for IsdA or for both IsdA and IsdB.

Two of three antibodies derived from IsdB immunization generated disease protection. These antibodies (3D8 and 4H7) bound with equal affinity to IsdA and IsdB, interfered with heme-iron binding to both IsdA and IsdBC. However, only one of these antibodies, 3D8, also triggered the killing of staphylococci in blood. A second IsdB MAb, 4H7, also interfered with heme binding to both IsdA and IsdBC. In contrast to 3D8, this MAb caused a more moderate reduction in bacterial load of abscess lesions and in the ability to reduce the survival of staphylococci in blood. One IsdB antibody did not cross-react with IsdA and did not affect heme binding, however this antibody also had no biological value in protecting against staphylococcal disease (2A9). Thus, antibodies—generated via either IsdA or IsdB immunization—that bind to both IsdA and IsdB afford disease protection. It is not known whether antibodies that bind only to IsdB, not to IsdA, can also protect against disease and trigger staphylococcal killing in mouse blood.

As discussed above all antibodies that blocked heme-binding of IsdA or IsdBc also interfered with heme binding of the other NEAT-domain protein. Further, all of the antibodies with heme-binding inhibition afford protection against staphylococcal disease. Thus, while the ability of MAbs to induce staphylococcal killing in blood is not correlated with disease protection, the ability to block heme-iron transport displays positive correlation. Additionally, mapping studies with IsdA deletion mutants, spanning the length of the protein, indicate regions near or within the NEAT domain are important for binding, and as these the regions that, when removed, result in lowered or no binding, are not consecutive, this strongly supports that all MAb epitopes are conformational. Further mapping evidence to support a functional role for the MAbs described here in blocking heme-iron uptake is based on removal of regions 130-209 resulting in decreased binding for many of the protective monoclonals (Table 4). This region contains Tyr166 which has recently been proposed to play a key role in transfer of heme between the different Isd system components, each protein possessing a similar tyrosine residue and coordinating histidine residues. Perhaps antibodies that bind to this similar conformational region on both IsdA and IsdB interfere with passage of heme across the cell envelope. Removal of residues 90-129 resulted in increased binding. This this region represents a portion of the protein, that in the crystal structure, would potentially limit antibody access Tyr166 and nearby histidines thought to be important for heme coordinating (Grigg et al., 2011). These antibodies and the discovery that they interfere with Isd-mediated heme transport provides essential insight into the development of immune therapeutics and vaccines for staphylococcal diseases where the ability to block heme-binding and heme-iron transport into bacteria can be used for the development of assays that serve as a correlate for protective immunity in vaccinated individuals. Finally, as both IsdA and IsdB can induce the formation of antibodies that block heme-iron transport, each of these two proteins should function as a valuable antigen for the development of human vaccines.

CDR Sequencing of IsdA and IsdB MAbs

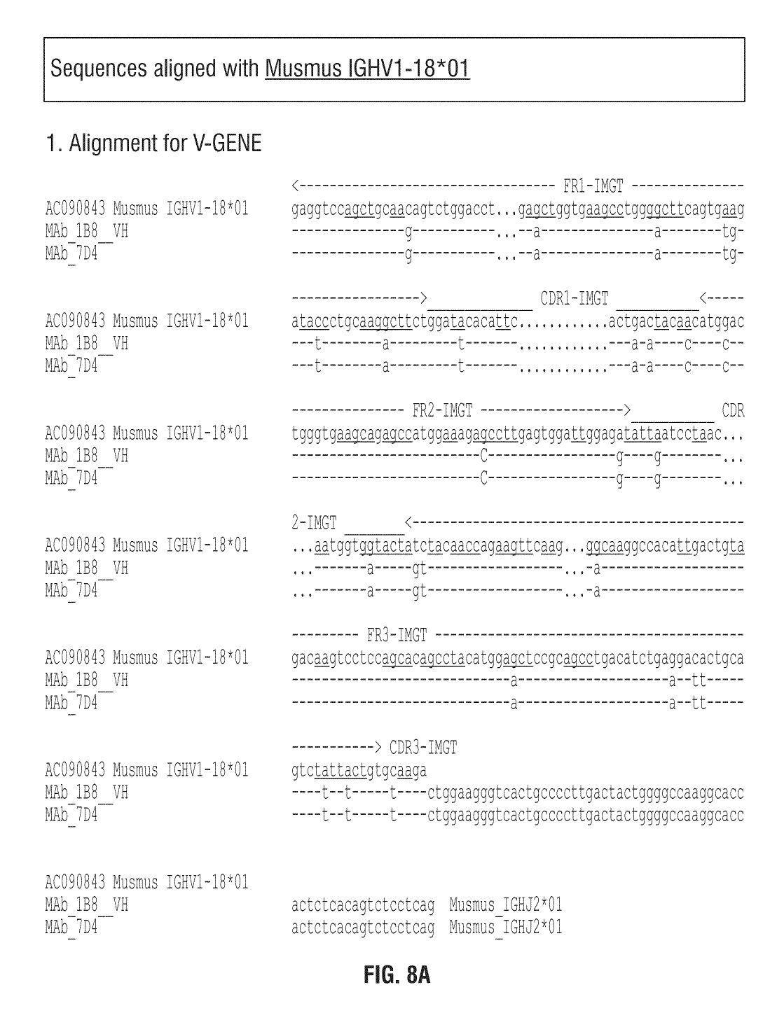

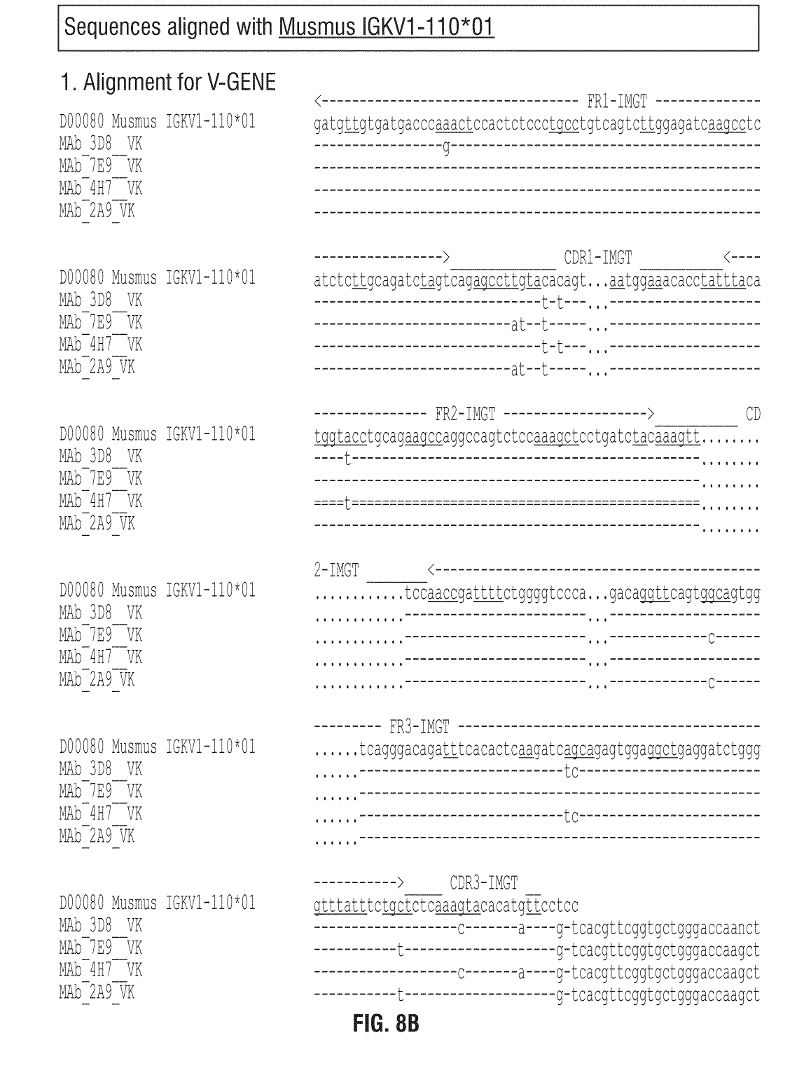

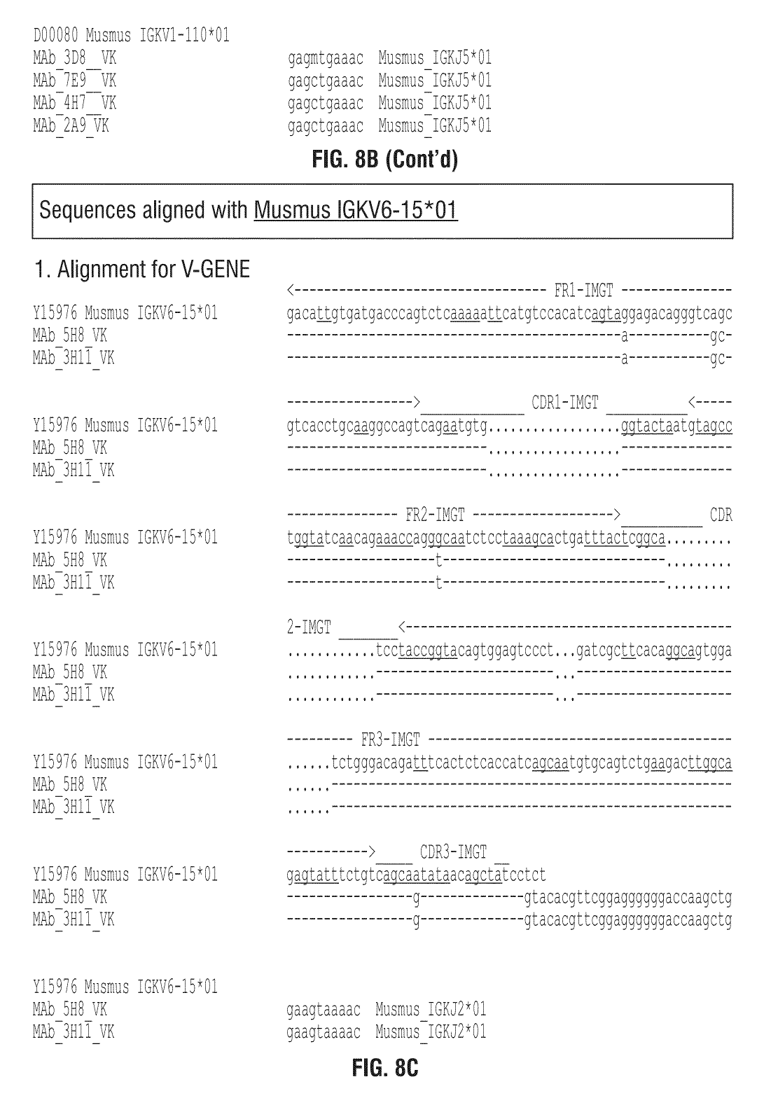

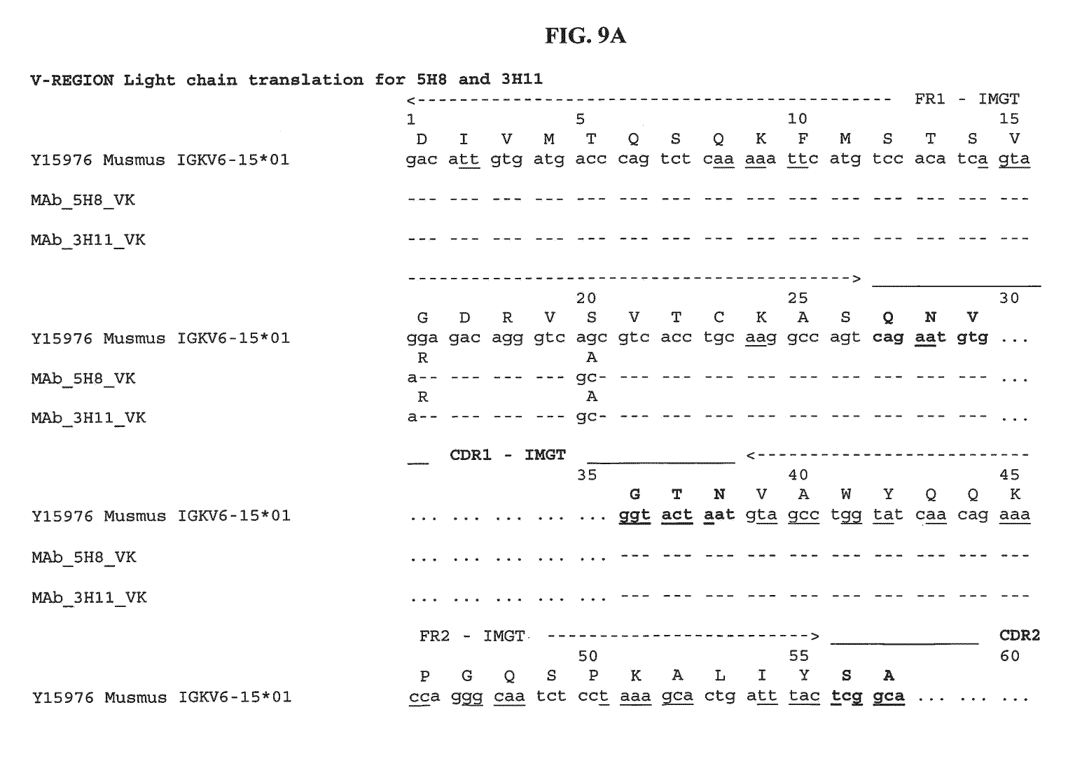

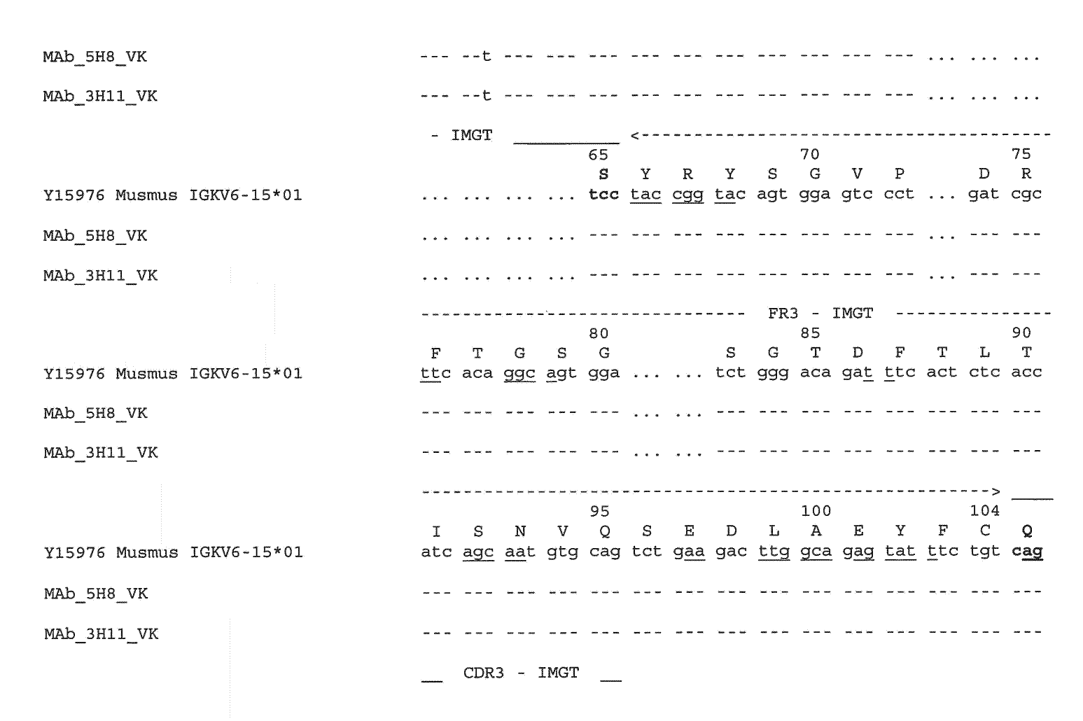

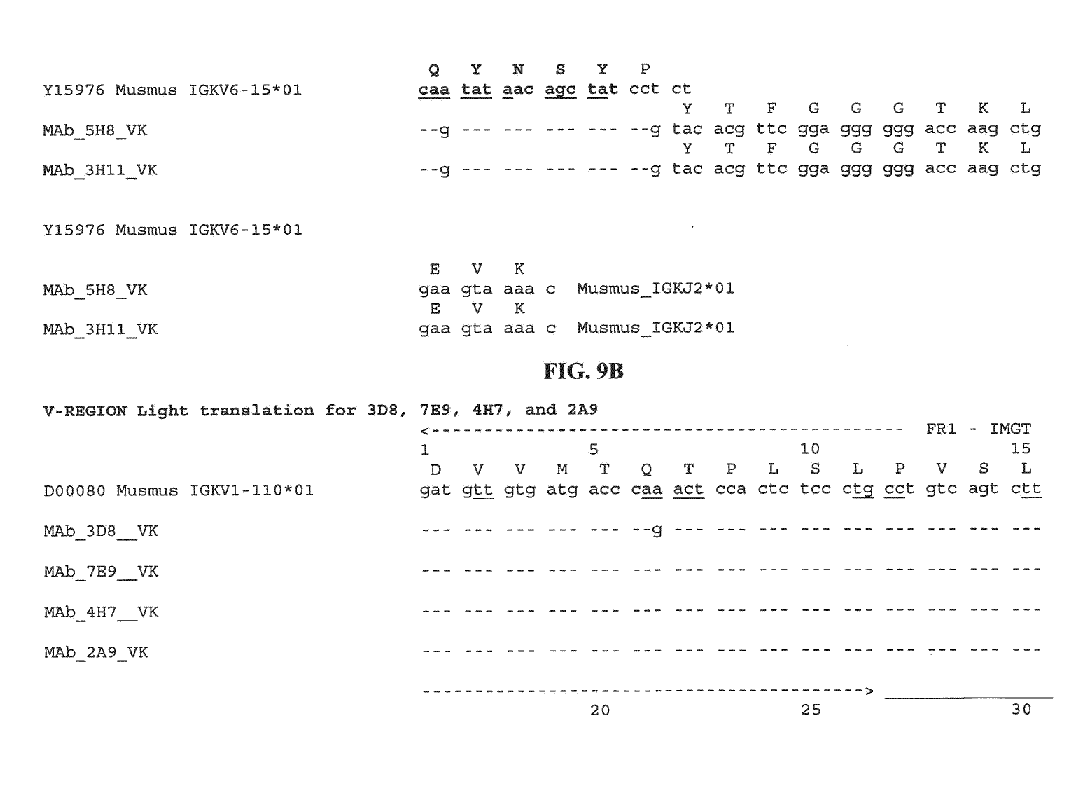

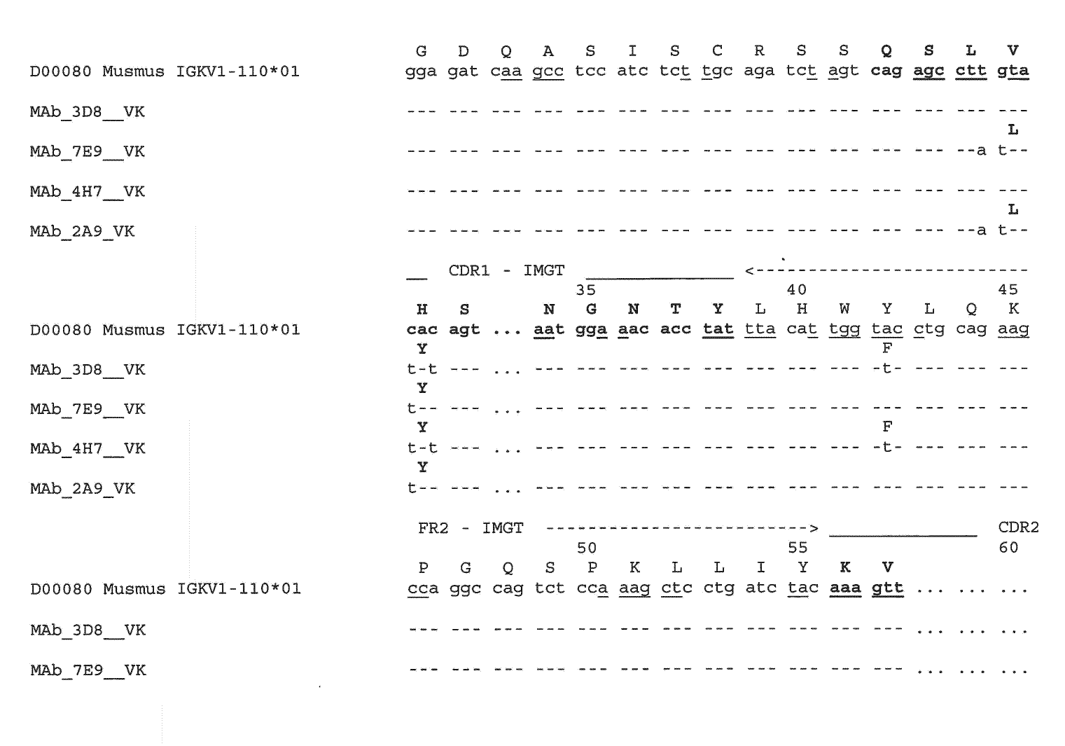

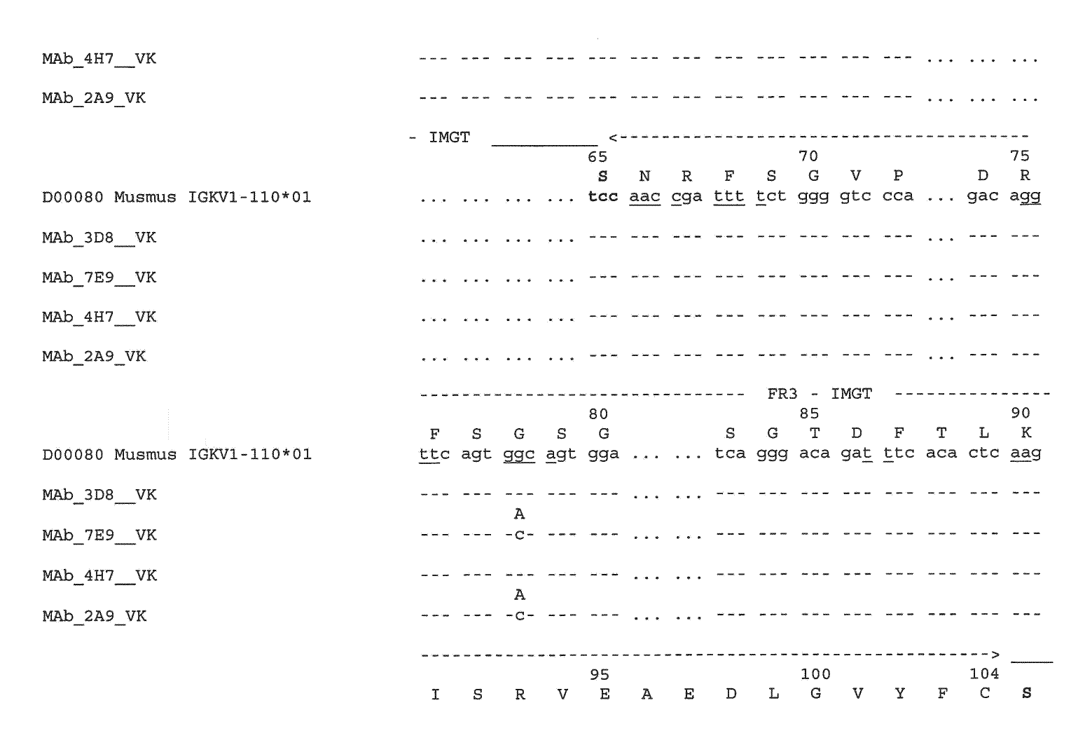

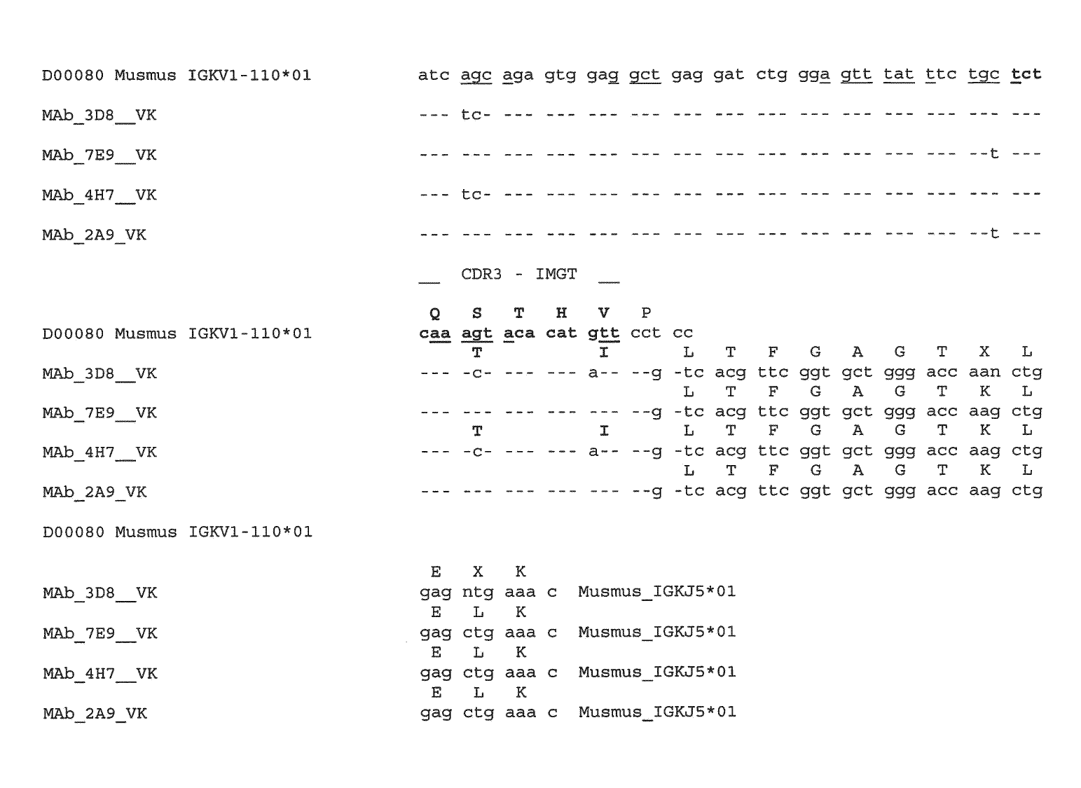

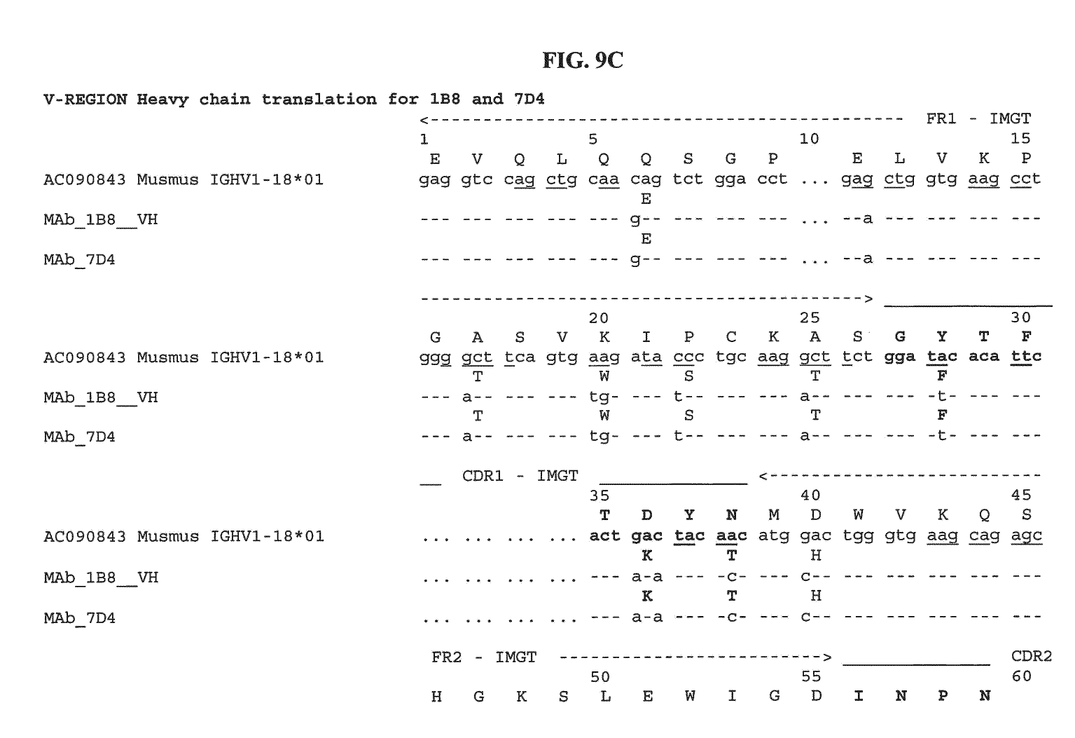

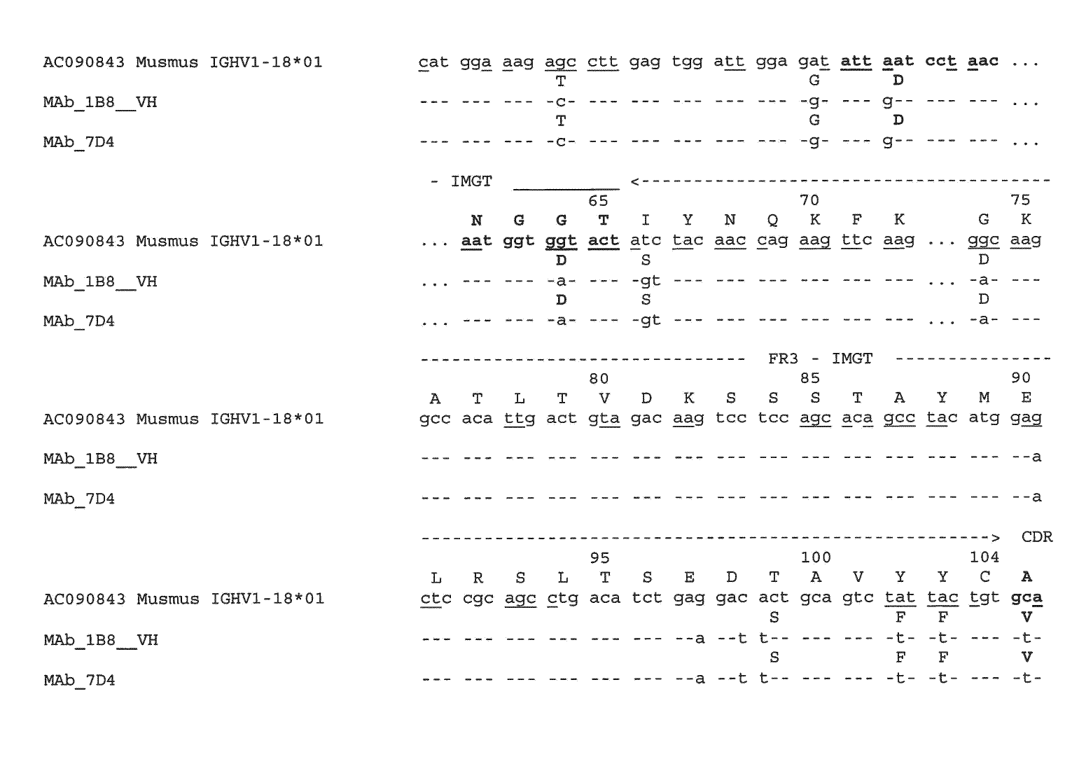

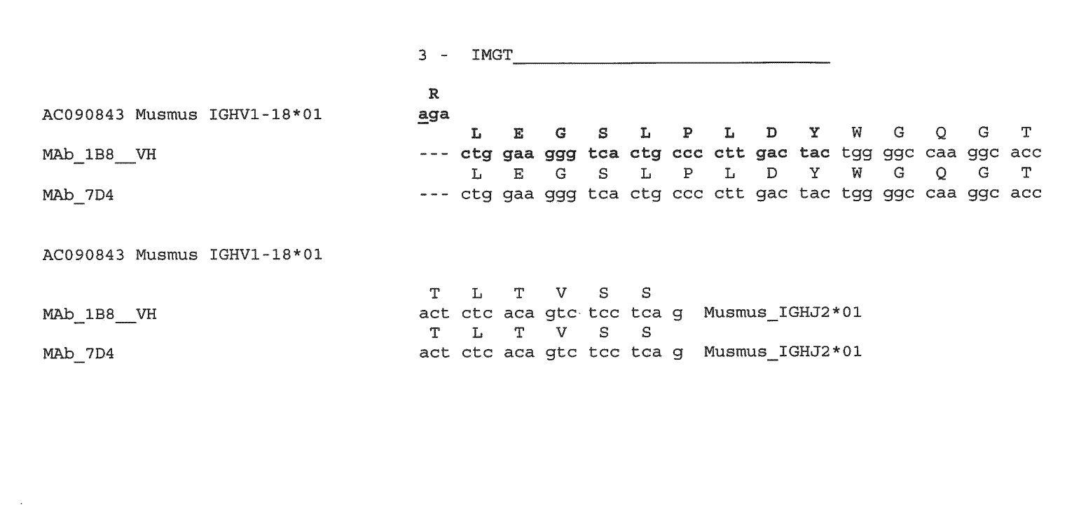

Total RNA from MAb hybridoma cells was isolated via standard protocol. Briefly, cells were washed in cold PBS and resuspended in Trizol. 20% Chloroform was added, mixed and incubated at room temperature for three minutes. Samples were centrifuged at 10K×g for fifteen minutes at 4° C. The aqueous layer was collected and washed with 70% isopropanol. RNA was pelleted by centrifugation and washed with 75% DEPC-ethanol. Pellets were dried and dissolved in DEPC. cDNA was synthesized and amplified by RT-PCR with Ig Primer sets designed to amplify the Ig variable regions. Independent primers and primers from Novagen were used which were designed to enable amplification from Ig conserved regions adjacent to the V_{H} and V_{L} hypervariable complementarity defining regions (CDRs). Positive products were sequenced and analyzed using IMGT vquest (at URL imgt.cines.fr/IMGT_vquest). Sequences for CDR 1, 2, and 3 for both V_{H} and V_{L} chains were obtained for MAbs 4B9, 5H8, 4H7, and 3H11. Sequences for CDR 1, 2, and 3. V_{L} chain sequences for CDR 1, 2, and 3 were obtained for MAbs 3D8, 7E9, and 2A9 while sequences for V_{H} chains were obtained for 1B8 and 7D4. Following Ig gene alignment analysis, three groups of MAbs were found to share sequence similarity. Isd MAbs 1B8 and 7D4 shared the same V,D and J sequences for the V_{H} genes (FIG. 8A). Isd MAbs 3D8, 7E9, 4H7 and 2A9 shared the same V and J sequences for the V_{L} genes (FIG. 8B) and the third group included 5H8 and 3H11 which shared the same V and J sequences for their respective V_{L} genes (FIG. 8C).

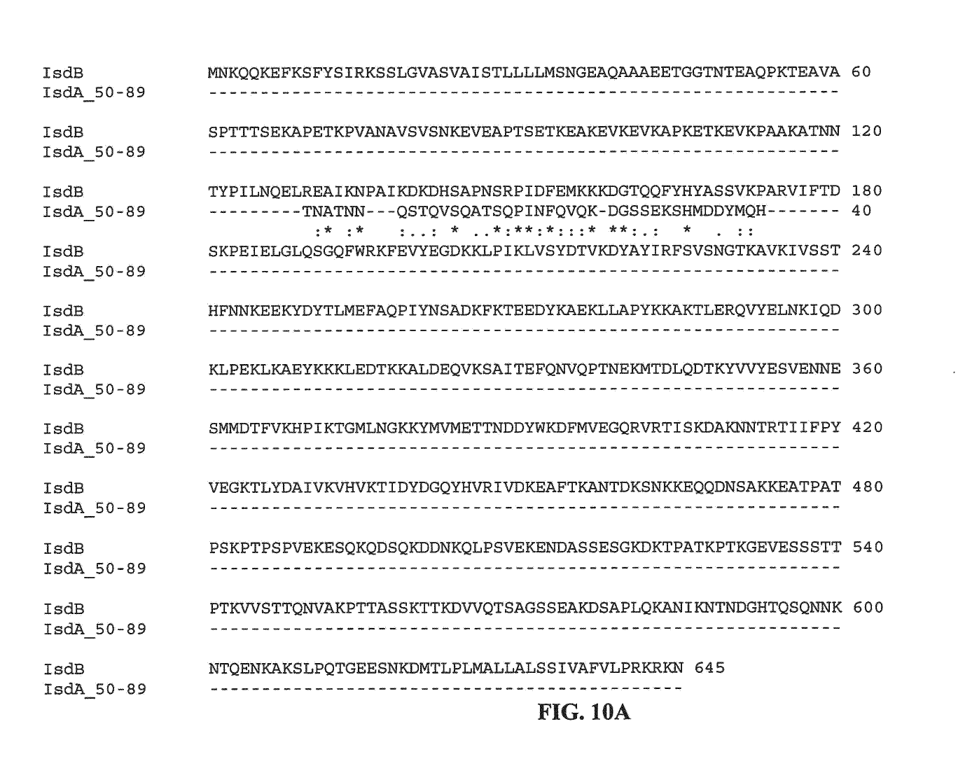

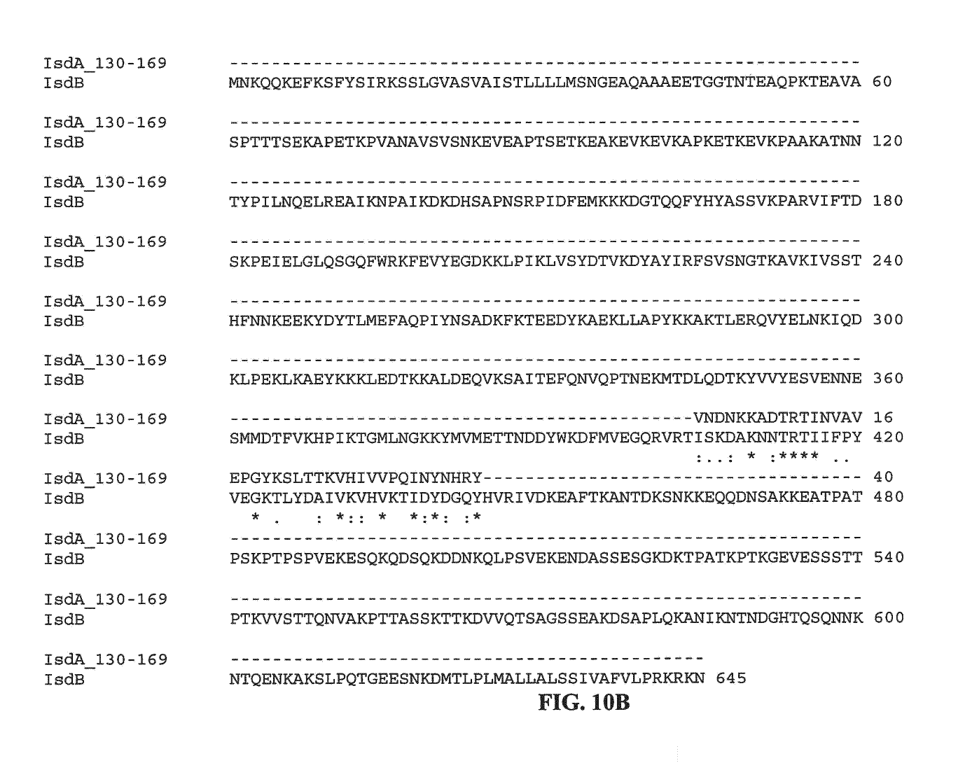

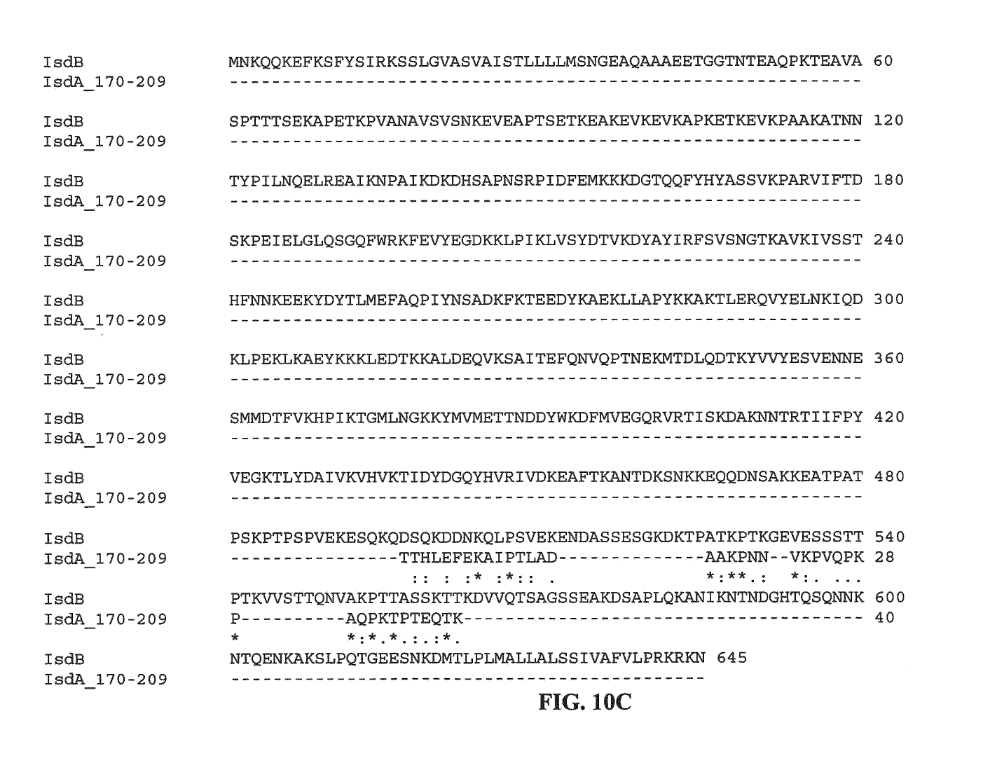

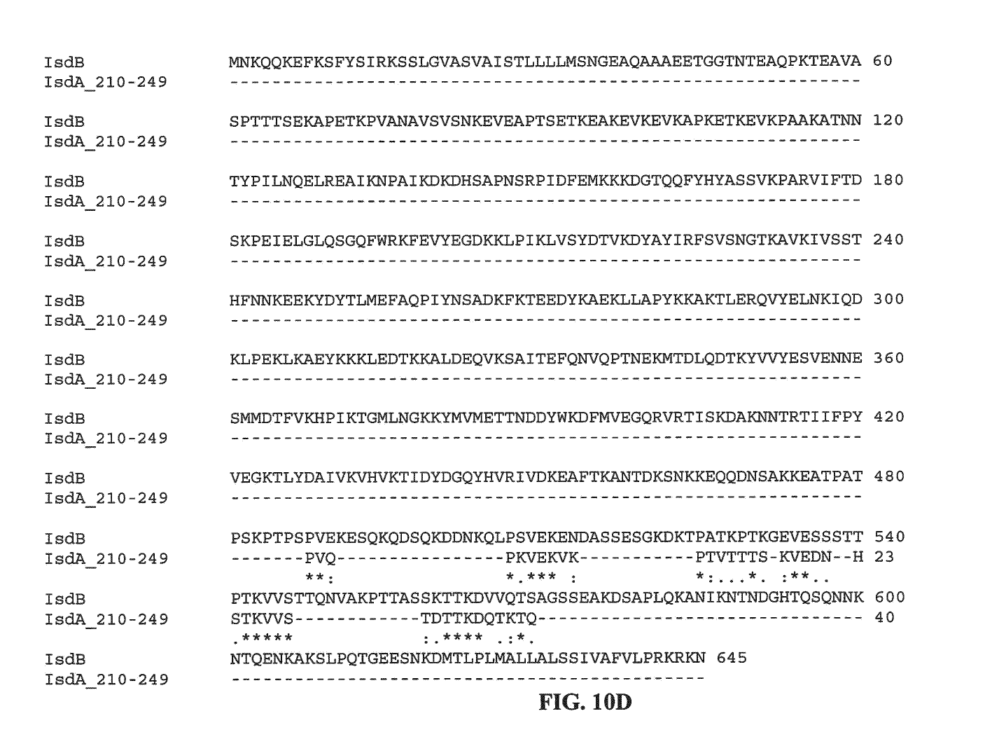

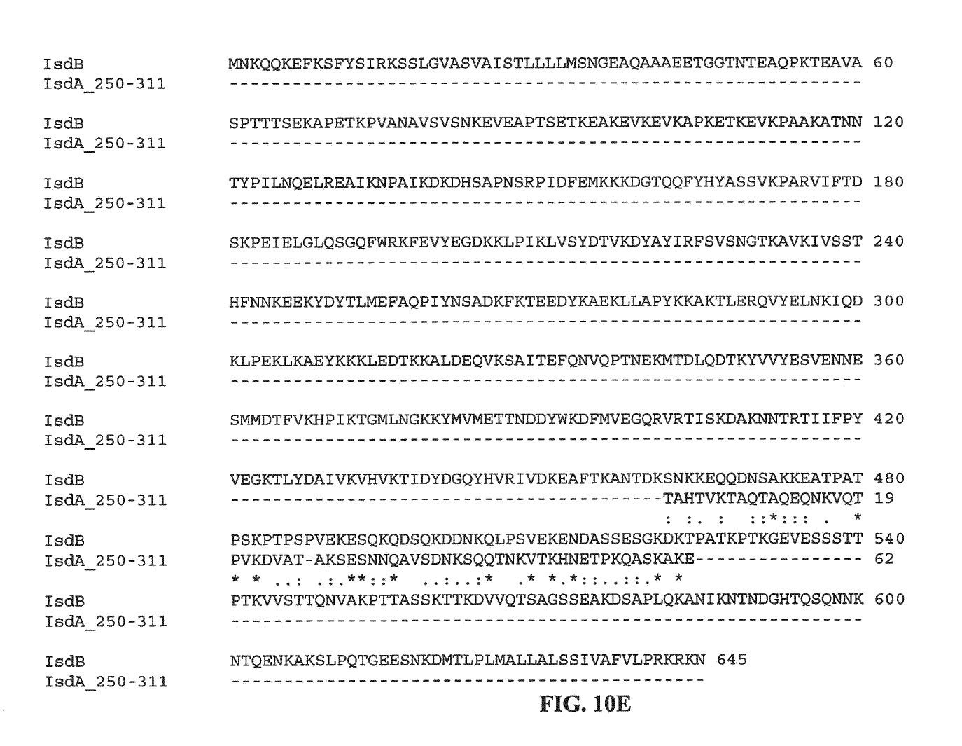

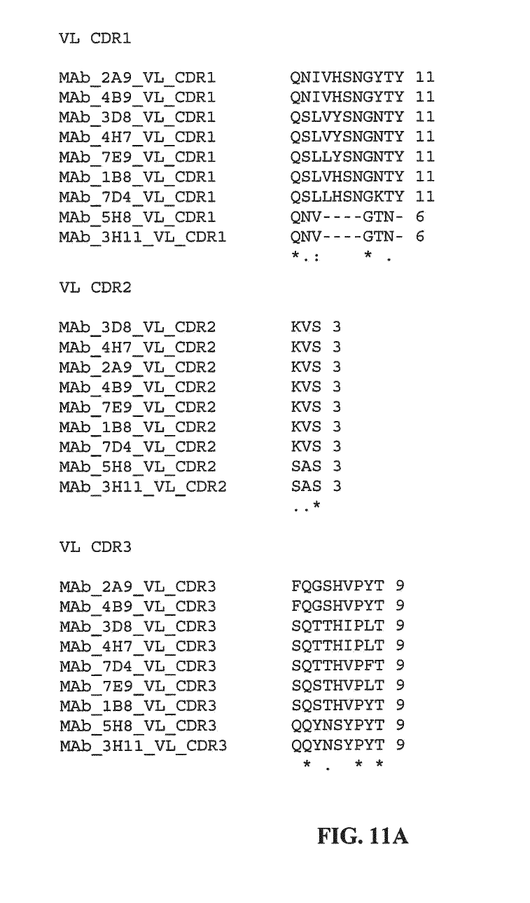

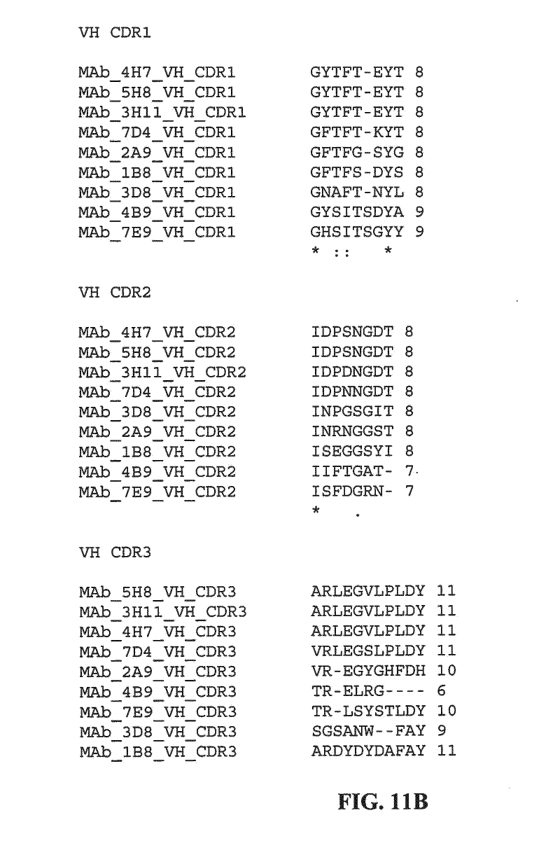

Sequence analysis was next undertaken to identify determinants that render antibody specific to IsdA or IsdB or cross-reactive with the two antigens. Regions of amino acid homology between IsdA and IsdB were assessed by aligning various regions of the proteins to identify putative epitopes recognized by cross reacting antibodies (see, e.g., FIG. 10). Regions important for antibody binding appear to be amino acids corresponding to IsdA aa 50-89; 170-209 and to lesser extent aa 130-169; 210-249 and 250-311 (for the 3D8, 4H7, 4B9 and 6H4 only). Following additional sequencing of the V_{H} and V_{L} genes of IsdA/B-binding antibodies the sequences were aligned to identify similarities in the CDR sequences between antibodies (see FIG. 11A-B).

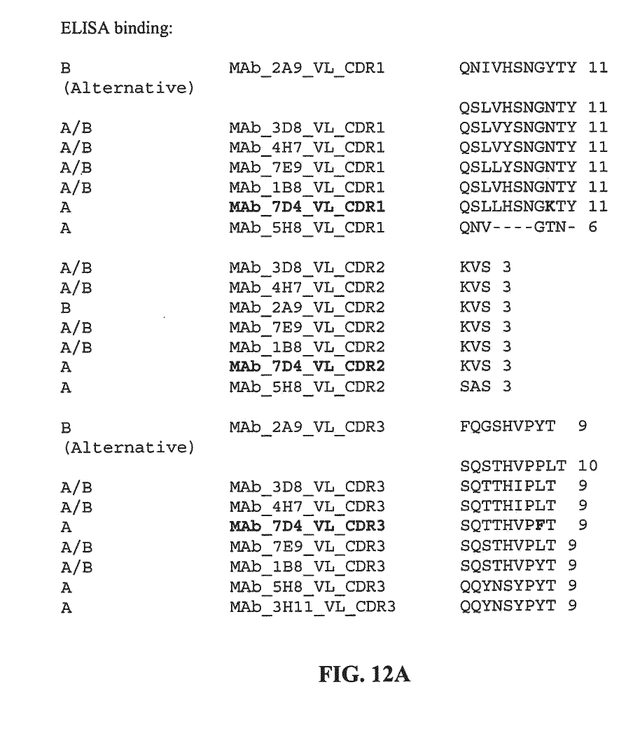

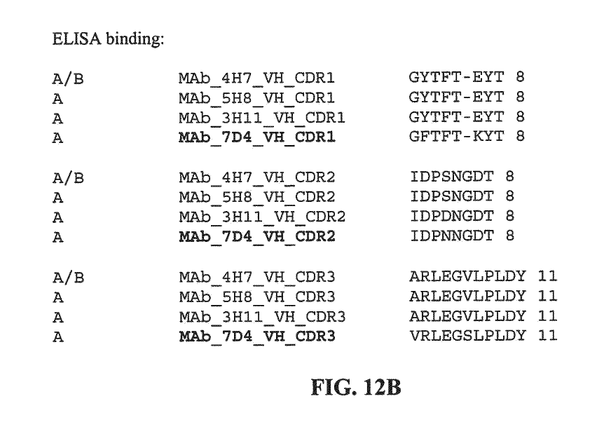

Likewise, alignments were generated to determine antibody determinants that render specificity for IsdA/IsdB. Mab 3H11 and 5H8 have identical VH and VL CDR amino acid sequences and are of the same Ig isotype-IgG2b. Comparison of the sequence of Mab 7D4 and the above two antibodies along with experimental data for each provides some insight into regions important for IsdA vs. IsdB binding specificity (FIG. 12A-B). The sequence data for 7D4 (which only binds IsdA, no cross-reactivity via ELISA to IsdB) and 4H7 (which binds both IsdA and IsdB) share significant sequence similarity in the VH CDR domains to 3H11 and 5H8 (which also only bind IsdA). The VL region of Mab 7D4 shares much more sequence similarity to antibodies which are also able to bind IsdB. Since 7D4 does not bind IsdB, despite sequence similarity in the light chain to antibodies that do, it is possible that the VH together with the slight amino acid changes in VL CDR1 (K) and CDR3 (F) provide IsdA-only specificity.

TABLE 4
Biochemical attributes and biological values of MAbs raised against IsdA and IsdB

| | | | Affinity^{d} | | | | | | | |
|---|---|---|---|---|---|---|---|---|---|---|
| MAb^{a} | Antigen^{b} | Class^{c} | IsdB_{FL} | IsdA_{FL} | Isd [symbol] | [symbol] | [symbol] | [symbol] | [symbol] | [symbol] |
| 3D8 | IsdB | IgG1 | 7.46 | 1.69 | 0 | 2.51 | 0.37 | 0 | 0.40 | 0.95 |
| 4H7 | IsdB | IgG1 | 3.09 | 3.73 | 5E−22 | 9.83 | 0.01 | 8E−30 | 0.08 | 0.34 |
| 2A9 | IsdB | IgG2a | 2.74 | 0.005 | 3E−25 | 0.01 | 0 | 1E−25 | 0 | 0.01 |
| 4B9 | IsdA | IgG2b | 1.14 | 0.11 | 0 | 1.74 | 0 | 0 | 2E−14 | 0.03 |
| 7E9 | IsdA | IgG2a | 5.36 | 8.08 | 0.372 | 9.91 | 2.74 | 0.03 | 10.04 | 8.44 |
| 1B8 | IsdA | IgG1 | 1.93 | 0.69 | 0 | 0.18 | 0 | 0 | 0 | 3.13 |
| 5H8 | IsdA | IgG2b | 0 | 6.75 | 0 | 7.74 | 0 | 0 | 0 | 8.62 |
| 7D4 | IsdA | IgG2a | 0 | 29.06 | 0 | 16.41 | 0 | 0.01 | 0.014 | 12.74 |
| 6A11 | IsdA | IgG2b | 10.04 | 4.52 | 0 | 9.56 | 0 | 1E−17 | 0 | 0.25 |
| 6A4 | IsdA | IgG2b | 6.0E−28 | 1.13 | 0 | 2.28 | 0 | 0 | 0 | 4.1 |
| 3H11 | IsdA | IgG2b | 0 | 16.96 | 4E−28 | 10.89 | 0.02 | 1E−16 | 5E−19 | 10.21 |
| 5F6 | IsdA | IgG2a | 14.14 | 9.18 | 0 | 14.27 | 0 | 0.005 | 2E−17 | 8.63 |
| 6H4 | IsdA | IgG1 | 0.005 | 3.11 | 7E−28 | 2.28 | 7E−14 | 1E−16 | 0.005 | 0.08 |
| 3E8 | IsdA | IgG2b | 0 | 9.57 | 0.01 | 10.59 | 0.01 | 7E−15 | 0.005 | 23.59 |

^{a}Mouse monoclonal antibodies were purified from isolated hybridoma clones
^{b}Antigen used to elicit mouse monoclonal antibodies
^{c}Immunoglobulin call and subclass of MAbs
^{d}Affinity was determined by ELISA as the association constant (Kₐ) in nM for each IsdA variant.

TABLE 5
MAb variable gene sequencing

| | | | V_{L}^{d} | | | V_{H}^{e} | | |
|---|---|---|---|---|---|---|---|---|
| MAb^{a} | Antigen^{b} | Class^{c} | CDR1 | CDR2 | CDR3 | CDR1 | CDR2 | CDR3 |
| 3D8 | IsdB | IgG1 | Yes | Yes | Yes | Yes | Yes | Yes |
| 4H7 | IsdB | IgG1 | Yes | Yes | Yes | Yes | Yes | Yes |
| 2A9 | IsdB | IgG2a | Yes | Yes | Yes | Yes | Yes | Yes |
| 4B9 | IsdA | IgG2b | Yes | Yes | Yes | Yes | Yes | Yes |
| 7E9 | IsdA | IgG2a | Yes | Yes | Yes | Yes | Yes | Yes |
| 1B8 | IsdA | IgG1 | Yes | Yes | Yes | Yes | Yes | Yes |
| 5H8 | IsdA | IgG2b | Yes | Yes | Yes | Yes | Yes | Yes |
| 7D4 | IsdA | IgG2a | Yes | Yes | Yes | Yes | Yes | Yes |
| 6A11 | IsdA | IgG2b | | | | | | |
| 6A4 | IsdA | IgG2b | | | | | | |
| 3H11 | IsdA | IgG2b | Yes | Yes | Yes | Yes | Yes | Yes |
| 5F6 | IsdA | IgG2a | | | | | | |
| 6H4 | IsdA | IgG1 | | | | | | |
| 3E8 | IsdA | IgG2b | | | | | | |

^{a}Mouse monoclonal antibodies were purified from isolated hybridoma clones
^{b}Antigen used to elicit mouse monoclonal antibodies
^{c}Immunoglobulin call and subclass of MAbs
^{d}Sequences obtained using primer pairs located within conserved regions adjacent to the hypervariable complementarity defining regions (CDRs) for the variable light chain genes (V_{L}). Yes indicates a positive sequencing event
^{e}Sequences obtained using primer pairs located within conserved regions adjacent to the hypervariable complementarity defining regions (CDRs) for the variable heavy chain genes (V_{H}). Yes indicates a positive sequencing event

Staphylococcus aureus Murine Renal Abscess Challenge Following Passive Transfer with mAb 3D8

Overnight cultures of staphylococcal strains (Newman wt or isdB-isogenic mutant) were diluted 1:100 into fresh TSB and grown until they reached an OD₆₀₀ of 0.4. Bacteria were centrifuged at 7,500×g, washed, and suspended in the same volume of 1×PBS. Six week-old female BALB/c mice (Charles River) were injected intra-peritoneal 4 hours prior to infection with 5 mg/kg body weight of IsdB mAb 3D8 (˜85 ug per mouse) or PBS control. Mice were injected retro-orbitally with 1×10⁷ CFU suspensions in 100 μl of PBS using cohorts of 10 mice. On the fifth day post infection, mice were killed by CO₂ asphyxiation and their kidneys excised and homogenized. Homogenates were plated by serial dilution and cfu recovery determined.

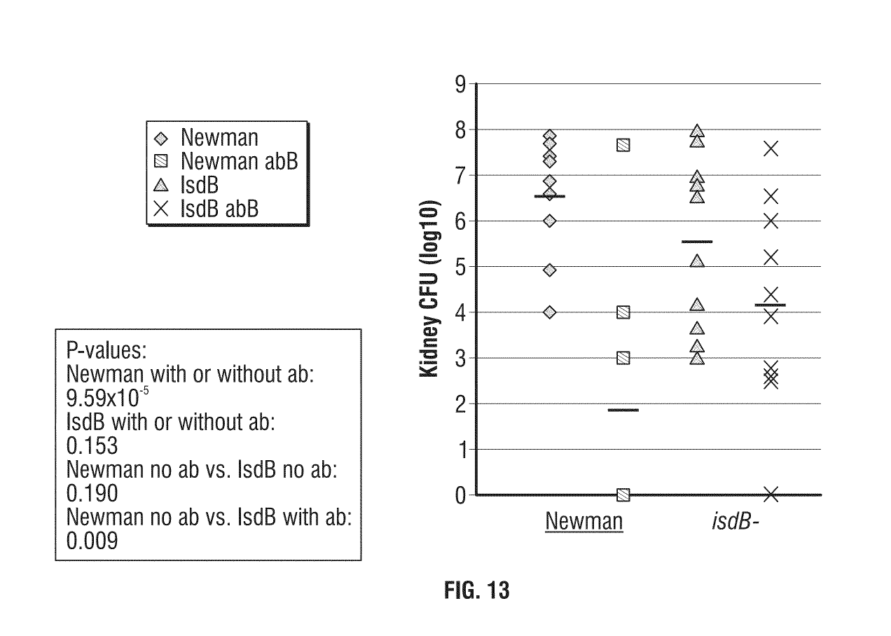

Following challenge with an isdB-isogenic mutant, the IsdB mAb 3D8 which exhibits cross reactivity to IsdA, provides an additional decrease in the cfu recovered following challenge (FIG. 13). Though the observed decrease was not statistically significant in this preliminary experiment compared to isdB-without antibodies, the trend suggests that the antibodies provide an additional benefit in the absence of IsdB. Additionally, animals challenged with the isdB-isogenic mutant had a nonsignificant reduction in cfu compared to wt challenge (without antibody), while mice given the 3D8 mAb and challenged with the isogenic mutant did display a significant reduction in cfu compared to wt. This result may be due to IsdA cross-reactivity (FIG. 13).

TABLE 6
Sequence Analysis of Mabs raised against IsdA and IsdB

| | | | Variable light Chain | | Variable heavy Chain | | |
|---|---|---|---|---|---|---|---|
| MAb^{a} | Antigen^{b} | Class^{c} | V | J | V | D | J |
| 4B9.25 | IsdB | IgG1 | Musmus IGKV1-117*01 | Musmus IGKJ2*01 | Musmus IGHV3-2*02 | Musmus IGHD3- | Musmus IGHJ2*01 |
| | | | | | | 1*01 | |
| 3D8.8 | IsdB | IgG1 | Musmus IGKV1-110*01 | Musmus IGKJ5*01 | Musmus IGHV1-54*01 F, or | Musmus IGHD1- | Musmus IGHJ3*01 |
| | | | | | Musmus IGHV1-54*02 F | 2*01 F | F |
| 7E9.11 | IsdB | IgG2a | Musmus IGKV1-110*01 | Musmus IGKJ5*01 | Musmus IGHV3-6*02 F | Musmus IGHD2- | Musmus IGHJ4*01 |
| | | | | | | 12*01 F | F |
| 1B8.8 | IsdA | IgG2b | Musmus IGKV1-110*01 | Musmus IGKJ2*01 | Musmus IGHV1-18*01, or | Musmus IGHD2- | Musmus IGHJ2*01 |
| | | | F | F | Musmus IGHV1-22*01 | 11*01 | |
| 5H8.9 | IsdA | IgG2a | Musmus IGKV6-15*01 | Musmus IGKJ2*01 | Musmus IGHV1-22*01 | Musmus IGHD2- | Musmus IGHJ2*01 |
| | | | | | | 4*01 | |
| 7D4.40 | IsdA | IgG1 | Musmus IGKV1-110*01 | Musmus IGKJ4*01 | Musmus IGHV1-18*01, or | Musmus IGHD2- | Musmus IGHJ2*01 |
| | | | F | F | Musmus IGHV1-22*01 | 11*01 | |
| 4H7.53 | IsdA | IgG2b | Musmus IGKV1-110*01 | Musmus IGKJ5*01 | Musmus IGHV1-22*01 | Musmus IGHD2- | Musmus IGHJ2*01 |
| | | | | | | 11*01 | |
| 3H11.47 | IsdA | IgG2b | Musmus IGKV6-15*01 | Musmus IGKJ2*01 | Musmus IGHV1-18*01, or | Musmus IGHD5- | Musmus IGHJ2*01 |
| | | | | | Musmus IGHV1-22*01 | 1*01 | |
| 2A9.21 | IsdA | IgG1 | Musmus IGKV1-110*01 | Musmus IGKJ5*01 | Musmus IGHV5-6-3*01 F | Musmus IGHD1- | Musmus IGHJ2*01 |
| | | | | | | 1*02 F | F |

^{a}Mouse monoclonal antibodies were purified from isolated hybridoma clones
^{b}Antigen used to elicit mouse monoclonal antibodies
^{c}Immunoglobulin call and subclass of MAbs
^{d}Hypervariable genes with similar sequences are color coded.

### Material and Methods

Bacterial Strains, Media and Growth Conditions.

S. aureus Newman (Baba et al., 2007) was grown in tryptic soy broth (TSB) at 37° C. The previously described isogenic isdA, isdB, isdC, and isdH mutants harboring the bursa aurelis mariner transposon were obtained from the Phoenix (φNξ) library (Bae et al., 2004. Transposon insertions were transduced into wild-type S. aureus Newman using bacteriophage φ85 and selected for on TSA plates with 10 μg ml⁻¹ erythromycin and 40 mM sodium citrate (Bae et al., 2004). For monitoring bacterial survival in human blood, S. aureus cultures were grown under low iron conditions, in chelex treated RPMI with 0.2 μM 2′-2-dipyridyl.

Rabbit Antibody Generation.

The coding sequences for IsdB_{N} were PCR-amplified with two primers, aactcgaggcagctgaagaaacaggt (SEQ ID NO:355) and aaggatcccacttgctcatctaaagc (SEQ ID NO:356), using S. aureus Newman template DNA. Sequences for IsdB_{C} were amplified with aactcgaggcmagatgagcaagtg (SEQ ID NO:357) and aaggatcctgattttgctttattttc (SEQ ID NO:358). PCR products were cloned into pET-15b or pGEX-2TK generating N-terminal His₆ tagged or N-terminal GST tagged recombinant proteins, respectively. Plasmids were transformed into BL21(DE3) or CA8000 and overnight cultures of transformants were diluted 1:100 into fresh media and grown at 37° C. to OD600 0.5, at which point cultures were induced with 1 mM isopropyl β-D-1-thiogalatopyranoside (IPTG) and grown for an additional three hours. Bacterial cells were sedimented by centrifugation, suspended in column buffer (50 mM Tris-HCl pH7.5, 150 mM NaCl) and disrupted with a French pressure cell. Lysates were cleared of membrane and insoluble components by ultracentrifugation at 40,000×g. Proteins in the soluble lysate were subjected to nickel-nitrilotriacetic acid (Ni-NTA) or GST affinity chromatography. Proteins were eluted in column buffer containing successively higher concentrations of imidazole (100-500 mM) or 30 mM reduced glutathione, respectively. Protein concentrations were determined by bicinchonic acid (BCA) assay (Thermo Scientific). For antibody generation, rabbits (Charles River Laboratories, 6 month old New-Zealand white, female) were immunized with 500 μg protein emulsified in Complete Freund's Adjuvant (Difco) by subscapular injection. For booster immunizations, proteins emulsified in Incomplete Freund's Adjuvant and injected 24 or 48 days following the initial immunization. On day 60, rabbits were bled and serum recovered.

Purified antigen (5 mg protein) was covalently linked to HiTrap NHS-activated HP columns (GE Healthcare). Antigen-matrix was used for affinity chromatography of 10-20 ml of rabbit serum at 4° C. Charged matrix was washed with 50 column volumes of PBS, antibodies eluted with elution buffer (1M glycine pH 2.5, 0.5 M NaCl) and immediately neutralized with 1M Tris-HCl, pH 8.5. Purified antibodies were dialyzed overnight against PBS at 4° C.

Passive Immunization.

Affinity purified antibodies prepared in PBS at 5 mg kg⁻¹ of experimental animal weight were delivered via interperitoneal injection into mice 24 hours prior to challenge with S. aureus. Animal blood was collected via periorbital vein puncture. Blood cells were removed with heparinized micro-hematocrit capillary tubes (Fisher) and Z-gel serum separation micro tubes (Sarstedt) were used to collect serum antibodies.

Purified antigens (IsdA, IsdB, IsdC, IsdB_{N} and IsdB_{C}) were coated onto MaxiSorp ELISA plates (NUNC) in 0.1 M carbonate buffer (pH 9.5) at 1 μg ml⁻¹ concentration overnight at 4° C. Plates were next blocked with 1% Bovine Serum Albumin (BSA) followed by incubation with serial dilutions of mouse sera (PBS, 1% BSA) for one hour. Plates were washed and incubated for an additional hour with secondary anti-mouse IgG HRP-conjugated rabbit antibody. Plates were washed and developed using OptEIA ELISA reagents (BD). Reactions were quenched with 1 M phosphoric acid and A450 readings were used to calculate half maximal IgG titers.

Mouse Renal Abscess.

Purified MAbs were injected into the peritoneal cavity of 6 week old female BALB/c mice (Charles River, cohorts of ten animals) at a concentration of 5 mg kg⁻¹ (typically 100 μg per animal of 20 g body weight). Overnight cultures of S. aureus Newman were diluted 1:100 into fresh TSB and grown for 2 hours at 37° C. Staphylococci were sedimented, washed and suspended in PBS at OD₆₀₀ of 0.4 (˜1×10⁸ CFU ml⁻¹). Inocula were enumerated by spreading sample aliquots on TSA and enumerating colony formation. BALB/c mice (Charles River Laboratories, 6 week old, female) were anesthetized via intraperitoneal injection with 100 mg ml⁻¹ of ketamine and 20 mg ml⁻¹ of xylazine per kilogram of body weight. Mice were infected by retro-orbital injection with 1×10⁷ CFU of staphylococci. On day 4 following challenge, mice were killed by CO₂ inhalation. Both kidneys were removed, and the staphylococcal load in one organ was analyzed by homogenizing its tissue with PBS, 1% Triton X-100. Serial dilutions of homogenate were spread on TSA and incubated for colony formation. The remaining organ was examined by histopathology. Briefly, kidneys were fixed in 10% formalin for 24 hours at room temperature. Tissues were embedded in paraffin, thin-sectioned, stained with hematoxylin-eosin, and inspected by light microscopy to enumerate abscess lesions. Animal experiments were performed in accordance with the institutional guidelines following experimental protocol review and approval by the Institutional Biosafety Committee (IBC) and the Institutional Animal Care and Use Committee (IACUC) at the University of Chicago.

Mouse Lethal Challenge.

Overnight cultures of S. aureus Newman were inoculated 1:100 into fresh TSB and grown for 2 hours at 37° C. Staphylococci were sedimented, washed and suspended in PBS. Staphylococci were diluted in PBS to OD600 of 0.4 (15-20×10⁸ CFU ml⁻¹). Each inoculum was quantified by spreading sample aliquots on TSA. BALB/c mice (6 week old, female, Charles River Laboratories) were anesthetized via intraperitoneal injection with 100 mg ml⁻¹ of ketamine and 20 mg ml⁻¹ of xylazine per kilogram of body weight. Animals were infected via retro-orbital injection with 15-20×10⁷ CFU of staphylococci. Infected animals were monitored for survival over a period of 10 days (240 hours).

Hemoglobin Binding Via GST Pulldown.

GST tagged proteins were purified from E. coli following IPTG induction as described. Cell lysates were applied to a 0.5 ml bed volume of glutathione sepharose beads and washed with 20 bed volumes of column buffer (50 mM TrisHCl, pH 7.5, 150 mM NaCl). Human hemoglobin (2 mg) was added to the column and washed with 10 bed volumes of column buffer. Bound proteins were eluted with 40 mM reduced glutathione, boiled in sample buffer, separated by SDS-PAGE, and stained with Coomassie.

Heme Binding.

GST-tagged IsdA and IsdB_{C} were purified as previously described (Kim et al., 2010) and incubated with individual IsdA or IsdB MAbs. Following incubation with hemin-chloride, absorbance spectroscopy was used to monitor heme binding (Skaar et al., 2004). Specifically, 3 μM protein was mixed with 3 μM of individual MAbs, incubated for 30 minutes at 25° C. and absorbance measured from 300-600 nm. Hemin chloride, 20 μM for IsdA or 30 μM IsdB_{C} was added and reactions incubated at 25° C. for an additional 10 minutes, followed by measurement of peak absorbance from 300-600 nm.

Heme Binding Via 3,3′,5,5′-tetramethylbenzidine (TMBZ).

Hemin-protein complex formation was determined by detection of heme dependent peroxidase activity, which turns the chromogenic compound 3,3′,5,5′-tetramethylbenzidine blue, as described previously (Allen et al., 2009; Stugard et al., 1989). Briefly, reactions with 1 μM IsdA, IsdB_{N}, or IsdB_{C} were incubated at room temperature for 30 minutes alone, in the presence of 3 μM αIsdB or 3 μM αV10, 0.5 μM hemin chloride was added and reactions proceeded for an additional hour. Proteins were directly separated by SDS-PAGE, without prior boiling or exposure to reducing agents. Gels were fixed for 1 hour in the dark at 4° C. in a prechilled solution of 0.25 M sodium acetate (pH 5.0), methanol, and water (6:3:1). The gels were then stained for 35 minutes in cold TMBZ staining solution, prepared just before use (12.6 mM TMBZ, 20% methanol, and 70% cold 0.25 M sodium acetate (pH 5.0). 30 mM hydrogen peroxide was added and incubated for an additional 30 minutes. Gels were washed with acetate buffered isopropanol (8:2) and dried.

Growth with Human Hemoglobin as the Sole Source of Iron.

S. aureus Newman overnight cultures grown in polypropelene 15 mL tubes were re-freshed four consecutive times in chelex treated RPMI+2′-2-dipyridyl to remove as much free iron as possible. For the final sub-culture, cells were grown for approximately eight hours to an OD₆₆₀ of 0.4, and washed 3 times in the same media. 2 μl of the cell suspension was inoculated into 100 μl of media with or without 5 μM freshly purified human hemoglobin into a 96 well micro-titre plate. Bacterial growth was monitored in a plate reader at OD₆₆₀ and OD₄₁₀ every 10 minutes for 16 hours alone or in the presence of 20 μg of IsdA, IsdB or isotype matched control IgG. Data are representative of 2 independent experiments, 3 replicates of each sample per experiment. Statistical significance was determined using the Kruskal Wallis analysis of variance with Prism graphpad software.

Surface Plasmon Resonance of IsdB Binding to Hemoglobin.

Hemoglobin binding to N-terminal His₆ tagged IsdA, IsdB, IsdB_{N} or IsdB_{C} was measured by surface plasmon resonance using a Biacore 3000 instrument (BIAcore AB, Uppsala, Sweden) at 18° C. A NTA biosensor chip was charged with 500 μM Ni²⁺ in HBS-P buffer (10 mM HEPES, pH 7.4, 0.15 M NaCl, 50 mM EDTA, 0.05% Tween 20) followed by protein immobilization at 200 nM at a flow rate of 10 μl min⁻¹. 5 μM Hemoglobin was injected at 20 μl min⁻¹ with a dissociation time of 180 seconds. Hemoglobin binding was calculated as Rmax hemoglobin/(RₘₐₓIsd protein/MW Isd protein). For antibody inhibition studies, 1 μM of αIsdB or αV10 were first injected at a flow rate of 20 μl min⁻¹ with a dissociation time of 180 seconds, followed by hemoglobin injection.

Monoclonal Antibodies Against IsdA and IsdB.

Mouse monoclonal antibodies were generated by the method of Fitch. On day 0, three 8-week-old BALB/c female mice, from Jackson Laboratory (Bar Harbor, Me.) were immunized intraperitoneally with 100 μg purified IsdA or IsdB antigen in phosphate buffered saline emulsified 1:1 with Complete Freund's Adjuvant (DIFCO). On days 21 and 42, mice were boosted by intraperitoneal injection with 100 μg purified IsdA or IsdB antigen emulsified 1:1 with Incomplete Freund's Adjuvant. On days 31 and 52, mice were bled and screened by ELISA on IsdA or IsdB coated Nunc MaxiSorp 96-well flat bottom plates. Seventy-nine days after the initial immunization, mice that showed strong immunoreactivity to antigen were boosted with 25 μg IsdA or IsdB in PBS. Three days later splenocytes were harvested and fused, according to standard methods, with the mouse myeloma cell line SP2/mIL-6, an interleukin 6 secreting derivative of SP2/0 myeloma cell line. Sups from resulting hybridomas were screened by ELISA and antigen-specific clones were subcloned, by limiting dilution, to produce monoclonal antibody-secreting hybridomas arising from single cells. Antibodies were purified from the culture supernatant of cell lines and stored at 1 mg ml⁻¹ in PBS.

IsdA and IsdB MAb Specificity and Affinity.

Poly-histidine tagged IsdA and IsdB were purified by affinity chromatography as described earlier (Kim et al., 2010). Proteins were used to coat Nunc MaxiSorp 96-well plates at a concentration of 1 μg ml⁻¹ PBS. Plates were washed and incubated with PBS-Tween and variable concentrations of MAbs. The affinity of MAbs to bind specific antigen, IsdA or IsdB, was measured as the concentration of bound/free antibody using secondary antibody-HRP conjugates and chemiluminescence for detection. Using this data, the association constant of antibody for antigen was calculated (Table 3). One IsdB-derived MAb (2A9) is specific for IsdB and does not crossreact with IsdA, however the other MAbs (3D8 and 4H7) bind equally well to both IsdA and IsdB. As the heme-iron binding NEAT domains represent the most closely related sequences between IsdA and IsdB, these data suggest that MAbs 3D8 and 4H7 may bind to these domains. Six IsdA-derived MAbs—5H8, 7D4, 6A4, 3H11, 6H4 and 3E8—are specific for IsdA and do not cross-react with IsdB. In contrast, MAbs 4B9, 7E9, 1B8, 6A11, and 5F6 do exhibit cross reactivity and bind to both IsdA and IsdB.

ELISA.

Poly-histidine tagged IsdA and IsdB were purified by affinity chromatography as described previously (Kim et al., 2010). Purified proteins were used to coat Nunc MaxiSorp 96-well plates at a concentration of 1 μg-ml⁻¹ 0.1 M carbonate buffer (pH 9.5 at 4° C.) overnight. Plates were washed three times and blocked with 1% BSA in PBS-Tween followed by incubation with varying concentrations of individual MAbs (six concentrations each, five-fold dilutions from 10 μg·ml⁻¹ to 3.2 ng·ml⁻¹) for one hour at room temperature. Plates were washed three times followed by incubation with anti-mouse HRP-conjugated secondary antibody for 1 hour, washed three times and developed using OptEIA reagent (BD Biosciences). Reactions were quenched with 1 M phosphoric acid and A₄₅₀ readings were used to calculate approximate K_{A}'s of individual MAbs for IsdA, IsdB, IsdB_{C}, IsdB_{N}, or IsdA deletion mutants, (IsdA-1_{FL50-311}, IsdA-2_{Δ50-89}, IsdA-3_{Δ90-129}, IsdA-4_{Δ130-169}, IsdA-5_{Δ170-209}, IsdA-6_{Δ210-249}, IsdA-7_{Δ250-311}).

### REFERENCES

The following references, to the extent that they provide exemplary procedural or other details supplementary to those set forth herein, are specifically incorporated herein by reference.
U.S. Pat. No. 3,791,932
U.S. Pat. No. 3,949,064
U.S. Pat. No. 4,174,384
U.S. Pat. No. 4,338,298
U.S. Pat. No. 4,356,170
U.S. Pat. No. 4,367,110
U.S. Pat. No. 4,372,945
U.S. Pat. No. 4,452,901
U.S. Pat. No. 4,474,757
U.S. Pat. No. 4,554,101
U.S. Pat. No. 4,578,770
U.S. Pat. No. 4,596,792
U.S. Pat. No. 4,599,230
U.S. Pat. No. 4,599,231
U.S. Pat. No. 4,601,903
U.S. Pat. No. 4,608,251
U.S. Pat. No. 4,683,195
U.S. Pat. No. 4,683,202
U.S. Pat. No. 4,684,611
U.S. Pat. No. 4,690,915
U.S. Pat. No. 4,748,018
U.S. Pat. No. 4,800,159
U.S. Pat. No. 4,879,236
U.S. Pat. No. 4,952,500
U.S. Pat. No. 5,084,269
U.S. Pat. No. 5,199,942
U.S. Pat. No. 5,221,605
U.S. Pat. No. 5,238,808
U.S. Pat. No. 5,302,523
U.S. Pat. No. 5,310,687
U.S. Pat. No. 5,322,783
U.S. Pat. No. 5,384,253
U.S. Pat. No. 5,464,765
U.S. Pat. No. 5,512,282
U.S. Pat. No. 5,538,877
U.S. Pat. No. 5,538,880
U.S. Pat. No. 5,548,066
U.S. Pat. No. 5,550,318
U.S. Pat. No. 5,563,055
U.S. Pat. No. 5,563,055
U.S. Pat. No. 5,580,859
U.S. Pat. No. 5,589,466
U.S. Pat. No. 5,591,616
U.S. Pat. No. 5,610,042
U.S. Pat. No. 5,620,896
U.S. Pat. No. 5,648,240
U.S. Pat. No. 5,656,610
U.S. Pat. No. 5,702,932
U.S. Pat. No. 5,736,524
U.S. Pat. No. 5,780,448
U.S. Pat. No. 5,789,215
U.S. Pat. No. 5,801,234
U.S. Pat. No. 5,840,846
U.S. Pat. No. 5,843,650
U.S. Pat. No. 5,846,709
U.S. Pat. No. 5,846,783
U.S. Pat. No. 5,849,497
U.S. Pat. No. 5,849,546
U.S. Pat. No. 5,849,547
U.S. Pat. No. 5,858,652
U.S. Pat. No. 5,866,366
U.S. Pat. No. 5,871,986
U.S. Pat. No. 5,916,776
U.S. Pat. No. 5,922,574
U.S. Pat. No. 5,925,565
U.S. Pat. No. 5,928,905
U.S. Pat. No. 5,928,906
U.S. Pat. No. 5,932,451
U.S. Pat. No. 5,935,819
U.S. Pat. No. 5,935,825
U.S. Pat. No. 5,939,291
U.S. Pat. No. 5,942,391
U.S. Pat. No. 5,945,100
U.S. Pat. No. 5,958,895
U.S. Pat. No. 5,981,274
U.S. Pat. No. 5,994,624
U.S. Pat. No. 6,008,341
U.S. Pat. No. 6,288,214
U.S. Pat. No. 6,294,177
U.S. Pat. No. 6,651,655
U.S. Pat. No. 6,656,462
U.S. Pat. No. 6,733,754
U.S. Pat. No. 6,756,361
U.S. Pat. No. 6,770,278
U.S. Pat. No. 6,793,923
U.S. Pat. No. 6,814,971
U.S. Pat. No. 6,936,258
U.S. Patent Publn. 2002/0169288
U.S. Patent Publn. 2003/0153022
U.S. Patent Ser. 61/166,432
U.S. Patent Ser. 61/170,779
U.S. Patent Ser. 61/103,196
Allen and Schmitt. J. Bacteriol., 191:2638-2648, 2009.
Anavi, Sc. thesis from the department of Molecular Microbiology and Biotechnology of the Tel-Aviv University, Israel, 1998.
Ausubel et al., In: Current Protocols in Molecular Biology, John, Wiley & Sons, Inc, New York, 1996.
Baba et al. J. Bacteriol. 190:300-310, 2007.
Bae et al., Proc. Natl. Acad. Sci. USA, 101, 12312-12317, 2004.
Barany and Merrifield, In: The Peptides, Gross and Meienhofer (Eds.), Academic Press, NY, 1-284, 1979.
Burke et al., J. Inf. Dis., 170:1110-1119, 1994.
Carbonelli et al., FEMS Microbiol. Lett., 177(1):75-82, 1999.
Chandler et al., Proc. Natl. Acad. Sci. USA, 94(8):3596-601, 1997.
Chang et al., Mol. Cell. Biol., 9:2153, 1989.
Chatterjee et al., Proc. Natl. Acad. Sci. USA, 86:9114, 1989.
Chen and Okayama, Mol. Cell. Biol., 7(8):2745-2752, 1987.
Cheng et al. J. Exp. Med. submitted for review, 2009.
Cheng et al., FASEB J. 23:1-12, 2009.
Cocea, Biotechniques, 23(5):814-816, 1997.
Cook et al., Hum. Vaccin. 5: Epub ahead of print, 2009.
DeDent et al., EMBO J., 27(20):2656-68, 2008.
DeDent et al., J. Bacteriol., 189(12):4473-84, 2007.
Deisenhofer et al., Hoppe Seylers Z Physiol Chem., 359(8):975-85, 1978.
Deisenhofer, Biochemistry, 20(9):2361-2370, 1981.
Dryla et al., J. Bacteriol., 189:254-264, 2007.
Dryla et al., Mol. Microbiol., 49:37-53, 2003.
Emorl and Gaynes, Clin. Microbiol. Rev., 6:428-442, 1993.
Epitope Mapping Protocols In: Methods in Molecular Biology, Vol. 66, Morris (Ed.), 1996,
Fattom et al., Vaccine, 22:880-887, 2004.
Fechheimer, et al., Proc Natl. Acad. Sci. USA, 84:8463-8467, 1987.
Fraley et al., Proc. Natl. Acad. Sci. USA, 76:3348-3352, 1979.
Gomez et al., J. Morphol., 259(1):90-105, 2004
Gomez, J. Biol. Chem., 281(29):20190-6, 2006.
Gopal, Mol. Cell. Biol., 5:1188-1190, 1985.
Graham and Van Der Eb, Virology, 52:456-467, 1973.
Graille et al., Proc. Natl. Acad. Sci. USA, 97(10):5399-404, 2000.
Grigg et al., Mol. Microbiol., 63:139-149, 2007.
Grigg et al., J. Mol. Biol., 413:684-698, 2011.
Harland and Weintraub, J. Cell Biol., 101(3):1094-1099, 1985.
Harlow et al., Antibodies: A Laboratory Manual, Cold Spring Harbor Laboratory, Cold Spring Harbor, N.Y., Chapter 8, 1988.
Huston et al., In: Methods in Enzymology, Langone (Ed.), Academic Press, NY, 203:46-88, 1991.
Jensen, Acta Path. Microbiol. Scandin., 44:421-428, 1958.
Johnson et al., Methods in Enzymol., 203:88-99, 1991.
Kaeppler et al., Plant Cell Rep., 8:415-418, 1990.
Kaneda et al., Science, 243:375-378, 1989.
Kato et al, J. Biol. Chem., 266:3361-3364, 1991.
Kim et al., Vaccine EPub: ahead of press, 2010.
Kim et al., J. Exp. Med. in press, 2010.
Kohler and Milstein, Nature, 256:495-497, 1975.
Kuklin et al., Infect. Immun., 74:2215-2223, 2006.
Kyte and Doolittle, J. Mol. Biol., 157(1):105-132, 1982.
Lancefield, J. Exp. Med., 47:91-103, 1928.
Lancefield, J. Immunol., 89:307-313, 1962.
Lee, Trends Microbiol., 4(4):162-166, 1996.
Levenson et al., Hum. Gene Ther., 9(8):1233-1236, 1998.
Liu et al., J. Biol. Chem., 283:6668-6676, 2008.
Marraffini and Schneewind, J. Biol. Chem., 280:16263-16271, 2005.
Mazmanian et al., Proc. Natl. Acad. Sci. USA, 97:5510-5515, 2000.
Mazmanian et al., Proc. Natl. Acad. Sci. USA, 99:2293-2298, 2002.
Mazmanian et al., Science, 285:760-763, 1999.
Mazmanian et al., Science, 299:906-909, 2003.
Memaugh et al., In: Molecular Methods in Plant Pathology, Singh et al. (Eds.), CRC Press Inc., Boca Raton, Fla., 359-365, 1995.
Merrifield, Science, 232(4748):341-347, 1986.
Muryoi et al., J. Biol. Chem., 283:28125-28136s, 2008.
Nicolau and Sene, Biochim. Biophys. Acta, 721:185-190, 1982.
Nicolau et al., Methods Enzymol., 149:157-176, 1987.
O'Seaghdha et al., FEBS J., 273(21):4831-41, 2006.
Omirulleh et al., Plant Mol. Biol., 21(3):415-28, 1993.
Overheim et al., Infect. Immun., 73:5152-5159, 2005.
PCT Appln. WO 00/02523
PCT Appln. WO 00/12132
PCT Appln. WO 00/12689
PCT Appln. WO 00/15238
PCT Appln. WO 01/60852
PCT Appln. WO 2006/032472
PCT Appln. WO 2006/032475
PCT Appln. WO 2006/032500
PCT Appln. WO 2007/113222
PCT Appln. WO 2007/113223
PCT Appln. WO 94/09699
PCT Appln. WO 95/06128
PCT Appln. WO 98/57994
Pilpa et al., J. Biol. Chem., 284: 1166-1176, 2009.
Pilpa et al., J. Mol. Biol., 360:435-447, 2006.
Potrykus et al., Mol. Gen. Genet., 199(2):169-177, 1985.
Raedler et al., Clin. Vaccine Immunol., in press, 2009.
Rippe, et al., Mol. Cell. Biol., 10:689-695, 1990.
Sambrook et al., In: Molecular cloning, Cold Spring Harbor Laboratory Press, Cold Spring Harbor, N.Y., 2001.
Schneewind et al., Cell, 70(2):267-81, 1992.
Sharp et al. J. Biol. Chem. 282: 10625-10631, 2007.
Sjodahl, Eur. J. Biochem., 73(2):343-51, 1977.
Sjoquist et al., Eur. J. Biochem., 29:572-578, 1972.
Skaar et al., Microbes Infect. 6:390-397, 2004a.
Skaar et al., Science 305:1626-1628, 2004b.
Skaar et al., J. Biol. Chem., 279:436-443, 2004c.
Stewart and Young, In: Solid Phase Peptide Synthesis, 2d. ed., Pierce Chemical Co., 1984.
Stranger-Jones et al., Proc. Nat. Acad. Sci. USA, 103:16942-16947, 2006.
Stugard et al., Infect. Immun., 57:3534-3539, 1989.
Tam et al., J. Am. Chem. Soc., 105:6442, 1983.
Thammavongsa et al., J. Exp. Med., submitted for publication, 2009.
Tigges et al., J. Immunol., 156(10):3901-3910, 1996.
Torres et al., J. Bacteriol., 188:8421-8429, 2006.
Uhlen et al., J. Biol. Chem., 259(3):1695-702, 1984.
Vaughan, et al., Nat. Biotech., 16; 535-539, 1998.
Wong et al., Gene, 10:87-94, 1980.
Wu et al., J Biol Chem., 2004
Zhu et al. J. Biol. Chem. 283:18450-18460, 2008.

1. A recombinant and isolated antibody, or antigen-binding portion thereof, that binds to a Staphylococcal IsdA and/or a Staphylococcal IsdB polypeptide wherein said antibody competes for binding of the polypeptide with a 3D8, 4H7, 2A9, 4B9, 7E9, 1B8, 5H8, 7D4 and/or 3H11 monoclonal antibody.

2-3. (canceled)

4. The antibody, or antigen-binding portion thereof, of claim 1, wherein said antibody, or antigen-binding portion thereof, comprises:
(a) a light chain variable region CDR1 sequence comprising the amino acid sequence QZ₁Z₂Z₃Z₄SNGZ₅TY, wherein Z₁ is S or N; Z₂ is L or I; Z₃ is V or L; Z₄ is H or Y and Z₅ is Y, N or K;
(b) a light chain variable region CDR2 sequence comprising the amino acid sequence KVS;
(c) a light chain variable region CDR3 sequence comprising the amino acid sequence Z₆QZ₇Z₈HZ₉Z₁₀PZ₁₁T, wherein Z₆ is F or S; Z₇ is G, T or S, Z₈ is S or T, Z₉ is V or I, Z₁₀ is absent or, if present, is P and Z₁₁ is Y, L or F;
(d) a heavy chain variable region CDR1 sequence comprising the amino acid sequence GZ₁₂TFZ₁₃Z₁₄YZ₁₅, wherein Z₁₂ is Y or F; Z₁₃ is T, G or S, Z₁₄ is E, K, S or D, and Z₁₅ is T, G or S;
(e) a heavy chain variable region CDR2 sequence comprising the amino acid sequence IZ₁₆Z₁₇Z₁₈Z₁₉Z₂₀Z₂₁Z₂₂, wherein Z₁₆ is D, N or S; Z₁₇ is P, R or E, Z₁₈ is S, D or N; Z₁₉ is N or G; Z₂₀ is G or S; Z₂₁ is D, S or Y; and Z₂₂ is T or I; and
(f) a heavy chain variable region CDR3 sequence comprising the amino acid sequence Z₂₃RZ₂₄Z₂₅Z₂₆Z₂₇Z₂₈Z₂₉Z₃₀Z₃₁Z₃₂, wherein Z₂₃ is A or V; Z₂₄ is absent or, if present, is L or D; Z₂₅ is E or Y; Z₂₆ is G or D; Z₂₇ is V, S or Y; Z₂₈ is L, G or D; Z₂₉ is P, H or A; Z₃₀ is L or F; Z₃₁ is D or A; and Z₃₂ is Y or H.

5-14. (canceled)

15. The antibody, or antigen-binding portion thereof, of claim 1 which is an isolated, monoclonal antibody, or antigen-binding portion thereof.

16. The antibody, or antigen-binding portion thereof, of claim 1 which is a human, humanized or de-immunized antibody.

17. The antibody, or antigen-binding portion thereof, of claim 1, wherein said antibody, or antigen-binding portion thereof comprises (a) a heavy chain comprising said Vh CDR sequences, and a human hinge, CH1, CH2, and CH3 regions from an IgG1, IgG2, IgG3 or IgG4 subtype; and (b) a light chain comprising said Vl CDR sequences, and either a human kappa CL or human lambda CL.

18. The antibody, or antigen-binding portion thereof, of claim 1, wherein the light chain variable region is less than 99%, 98%, 97%, 95% or 90% identical to SEQ ID NO: 359, 363, 365, 367, 371, 377, 383, 387, 393 or 397.

19. The antibody, or antigen-binding portion thereof, of claim 1, wherein the heavy chain variable region is not identical to SEQ ID NO: 361, 369, 373, 375, 379, 381, 385, 389, 391 or 395.

20. The antigen-binding portion of claim 1 which is or which comprises a Fab′, a F(ab′)2, a F(ab′)3, a monovalent scFv, a bivalent scFV, or a single domain Ab.

21-23. (canceled)

24. The antibody, or antigen-binding portion thereof, of any one of claims 1-9, which binds to both a Staphylococcal IsdA and a Staphylococcal IsdB polypeptide.

25-31. (canceled)

32. A method of reducing Staphylococcus infection abscess formation comprising administering to a patient having or suspected of having a Staphylococcus infection an effective amount of an antibody, or antigen-binding portion thereof, according to claim 1.

33. The method of claim 1, further comprising administering a second antibody that binds a second Staphylococcal protein.

34. The method of claim 1, further comprising administering an antibiotic.

35. The method of claim 1, wherein the antibody is administered at a dose of 0.1 mg/kg to 5 mg/kg.

36. A method of treating a subject having or suspected of having a Staphylococcus infection comprising administering to a patient having or suspected of having a Staphylococcus infection an effective amount of an antibody, or antigen-binding portion thereof, according to claim 1.

37. (canceled)
